(19) United States
(12) Patent Application Publication
(10) Pub. No.: US 2008/0085515 A1
(43) Pub. Date: Apr. 10, 2008

(54) IDENTIFICATION OF MULTIPLE BIOLOGICAL (MICRO) ORGANISMS BY DETECTION OF THEIR NUCLEOTIDE SEQUENCES ON ARRAYS

(75) Inventors: Jose Remacle, Malonne (BE); Sandrine Hamels, Ways (BE); Nathalie Zammatteo, Gelbressee (BE); Isabelle Alexandre, Haltinne (BE); Francoise de Longueville, Natoye (BE)

Correspondence Address: KNOBBE MARTENS OLSON & BEAR LLP, 2040 MAIN STREET, FOURTEENTH FLOOR, IRVINE, CA, 92614, US

(73) Assignee: Eppendorf Array Technologies SA (EAT), Namur (BE)

(21) Appl. No.: 11/694,871

(22) Filed: Mar. 30, 2007

Related U.S. Application Data
(63) Continuation-in-part of application No. 10/056,229, filed on Jan. 23, 2002, now Pat. No. 7,202,026.
Application No. 10/056,229 is a continuation-in-part of application No. 09/817,014, filed on Mar. 23, 2001, now Pat. No. 7,205,104.

(30) Foreign Application Priority Data
Mar. 24, 2000 (EP) 00870055.1
Sep. 15, 2000 (EP) 00870204.5

(51) Int. Cl. C12Q 1/68; C12M 1/34

(52) U.S. Cl. 435/6; 435/287.2

(57) ABSTRACT

The present invention is related to a method for identifying and/or quantifying an organism or part of an organism in a sample by detecting a nucleotide sequence specific for said organism, among at least 4 other nucleotide sequences from other organisms or from parts of the organisms. The method includes the steps of: producing derived sequences from the organism nucleotide sequences by incorporation of at least one common sequence in said organism nucleotide sequences in order to obtain a partial homology between the said specific derived nucleotide sequences; amplifying said specific derived nucleotide sequences by PCR into double stranded target nucleotide sequences using a unique pair of primer(s), which recognize the common sequence of the derived sequences and which are capable of amplifying at least 4 other derived nucleotide sequences as to produce full-length target nucleotide sequences having between 60 and 800 bases; contacting said full-length target nucleotide sequences resulting from the amplifying step with at least 5 different single-stranded capture nucleotide sequences having between 55 and 800 bases, preferably between about 60 and about 450 bases, said single-stranded capture nucleotide sequences being covalently bound in an microarray to insoluble solid support(s) and said capture nucleotide sequences comprising a nucleotide sequence of at least 15 bases which is able to specifically bind to said full-length target nucleotide sequence without binding to said at least 4 other derived nucleotide sequences, and said specific sequence is separated from the surface of the solid support by a spacer comprising a nucleotide sequence of at least 40 bases in length; and detecting specific hybridization of said target nucleotide sequence to said capture nucleotide sequences present at specific locations.

### CROSS-REFERENCE TO RELATED APPLICATIONS

This is a continuation-in-part of U.S. patent application Ser. No. 10/056,229, filed Jan. 23, 2002, which is a continuation-in-part of U.S. patent application Ser. No. 09/817,014 filed Mar. 23, 2001, which claims priority to European Application Serial Number 00870055.1 filed on Mar. 24, 2000, and European Application Serial Number 00870204.5 filed on Sep. 15, 2000, the disclosures of all of which are incorporated herein by reference in their entireties.

### FIELD OF THE INVENTION

The present invention relates to the diagnosis and analytical assays and is related to a method and kit comprising reagents and means for the identification, detection and/or quantification of a large number of (micro)organisms of different groups (classes, family, genus, species, individual among other ones) by their identification or the identification of a component thereof on a same array.

The invention is especially suited for the simultaneous identification and/or quantification of groups and sub-groups of (micro)organisms or related genes present in the same biological sample.

The present invention also provides a two step method for detecting first for the presence of any of the search (micro)organisms followed by its identification.

The present invention is in the field of diagnosis and is related to a method and kit comprising reagents and means for the identification (detection and/or quantification) of different (micro)organisms among other ones having different nucleotide sequences by identification of their nucleotide sequences by hybridization on specific immobilized capture molecules after amplification by PCR

The invention is especially suited for the identification and/or quantification of different (micro)organisms of the same genus or family or for the detection and/or quantification of different genes in a specific (micro)organism present in a biological sample.

### DESCRIPTION OF THE RELATED ART

Identification of an organism or microorganisms can be performed based on the presence in their genetic material of specific sequences. Identification of a specific organism can be performed easily by amplification of a given sequence of the organism using specific primers and detecting or identifying the amplified sequence.

However, in many applications especially in diagnostic, possible organisms present in biological samples are numerous and belong to different families, genus, species, subspecies or even individuals. Amplifications of each of the possible organisms is difficult and expensive. A simple method is thus required for such multi-parametric, multi-levels analysis.

Amplification of a given sequence is performed by several methods such as the polymerase chain reaction (PCR) (U.S. Pat. Nos. 4,683,195 and 4,683,202), ligase chain reaction (LCR) (Wu and Wallace, 1989 Genomics 4:560-569) or the Cycling Probe Reaction (CPR) (U.S. Pat. No. 5,011,769), which are the most common. One particular way to detect the presence of a given sequence and thus of a particular organism is to follow the appearance of amplicons during the amplicon cycles. The method is called the real time PCR. A fluorescent signal appears when the amplifications are formed and the amplification is considered as positive when reaching a threshold.

Detecting the amplicons can also be performed after the amplification by methods based on the specific recognition of amplicons to complementary sequences. The first supports used for such hybridization were the nitrocellulose or nylon membranes. However, the methods were miniaturized and new supports such as conducting surfaces, silica, and glass were proposed together with the miniaturization of the detection process. Micro-arrays or DNA Chips are used for multiple analysis of DNA or RNA sequences either after an amplification step or after a reverse transcription into a cDNA. The target sequences to be detected are labeled during the amplification or copying step and are then detected and possibly quantified on arrays. The presence of a specific target sequence on the arrays is indicative of the presence of a given gene or DNA sequence in the sample and thus of a given organism which may then be identified. The problem of detection becomes difficult when several sequences are homologous to each other, but have to be specifically discriminated upon the same array. This technical problem is the condition to use arrays for many diagnostic purposes since organisms or micro-organisms of interest are often very similar to others on a taxonomic basis and have almost identical DNA sequences.

The Company Affymetrix Inc. has developed a method for direct synthesis of oligonucleotides upon a solid support, at specific locations by using masks at each step of the processing. Said method comprises the addition of a new nucleotide on a growing oligonucleotide in order to obtain a desired sequence at a desired location. This method is derived from the photolithographic technology and is coupled with the use of photoprotective groups, which are released before a new nucleotide is added (EP-A1-0476014, U.S. Pat. No. 5,445,934, U.S. Pat. No. 5,143,854 and U.S. Pat. No. 5,510,270). However, only small oligonucleotides are present on the surface, and said method finds applications mainly for sequencing or identifying a pattern of positive spots corresponding to each specific oligonucleotide bound on the array. The characterization of a target sequence is obtained by comparison of such pattern with a reference. Said technique was applied to the identification of Mycobacterium tuberculosis rpoB gene (WO97/29212 and WO98/28444), wherein the capture nucleotide sequence comprises less than 30 nucleotides and from the analysis of two different sequences that may differ by a single nucleotide (the identification of SNPs or genotyping). Small capture nucleotide sequences (having a length comprised between 10 and 20 nucleotides) are preferred since the discrimination between two oligonucleotides differing in one base is higher, when their length is smaller.

The method is complicated by the fact that it cannot directly detect amplicons resulting from genetic amplification (PCR). A double amplification is performed with primer(s) bearing a T3 or T7 sequences and then a reverse transcription with a RNA polymerase. These RNA are cut into pieces of about 40 bases before being detected on an array (example 1 of WO 97/29212). Each sequence requires the presence of 10 capture nucleotide sequences and 10 control nucleotide sequences to be identified on the array. The reason for this complex procedure is that long DNA or RNA fragments hybridize very slowly on small oligonucleotide capture nucleotide sequences present on the surface. Said methods are therefore not suited for the detection of homologous sequences, since the homology varies along the sequences and so part of the pieces will hybridize on the same capture nucleotide sequences. Therefore, a software for the interpretation of the results is incorporated in the method for allowing interpretation of the obtained data. The main reason not to perform a single hybridization of the amplicons on the array is that the amplicons will rehybridize in solution much faster than hybridize on the small capture nucleotide sequences of the array.

One consequence of such constraints is that polynucleotides are analyzed on oligonucleotides based arrays, only after being cut into oligonucleotides. Said methods are therefore not suited for the detection of homologous sequences since the homology varies along the sequences and so part of the pieces could hybridize on the same capture probes. For gene expression array which is based on the detection of cDNA copy of the mRNA, the problem still exist but is less acute since the cDNA is single stranded. The fragments are also cut into smaller species and the method requires the use of several capture oligonucleotide sequences in order to obtain a pattern of signals which attest the presence of a given gene. Said cutting also decreases the number of labeled nucleotides, and thus reduces the obtained signal. In the case of cDNA analysis, the use of long capture polynucleotide sequences gives a much better sensitivity to the detection. In many gene expression applications, the use of long capture nucleotide sequences is not a problem, when cDNAs to be detected originate from genes having different sequences, since the difference in the sequence is sufficient in order to avoid cross reactions between them even on a sequence longer than 100 bases so that polynucleotides can be used as capture nucleotide sequences. Long capture nucleotide sequences give the required sensitivity but they will hybridize to other homologous sequences.

The main reason not to perform a single hybridization of the amplicons on the array is that the amplicons will rehybridize in solution much faster than hybridize on the small capture nucleotide sequences of the array.

However, for gene expression array which is based on the cDNA copy of mRNA the same problem is encountered when using small capture probe arrays: the rate of hybridisation is low. Therefore, the fragments are cut into smaller pieces and the method requires the use of several capture nucleotide sequences in order to obtain a pattern of signals which attest to the presence of a given gene (WO97/10364 and WO97/27317). Said cutting also decreases the number of labeled nucleotides, and thus reduces the obtained signal. In this case, the use of long capture nucleotide sequences gives a much better sensitivity to the detection. In many gene expression applications, when cDNA to be detected originates from genes having different sequences, the use of long capture probes is not a problem, since there is no cross-reactions between them. Long capture nucleotide sequences give the required sensitivity, however, they will hybridize to other homologous sequences.

The detection of Single Nucleotide Polymorphism in the DNA is just one particular aspect of the detection of homologous sequences. The use of arrays has been proposed to discriminate two sequences differing by one nucleotide at a particular location of the sequence. Since DNA or RNA sequences are in low copy numbers, their sequences are first amplified so that double stranded sequences are analyzed on the array. Several methods have been proposed to detect such a base change in one location. The document WO 97/31256 proposes the use of two oligonucleotide sequences: the first one with a part specific and a part addressable, the second one with a part specific and a part labeled. After ligation in solution, the product is immobilized on an array with capture nucleotide sequences with a least a part complementary of the addressable part. The detection of SNP is the basis for polymorphism determination of individual organism, but also for its genotyping, since the genome of individuals differ from each other in the same species or subspecies by said SNPs. The presence of a particular SNP affect the activities of enzymes like the P450 and make them more or less active in the metabolism of a drug.

The capture oligonucleotide present on the array can also be used as primers for extension once the target nucleotide hybridized. The document WO 96/31622 proposes to identify a nucleotide at a given location upon a sequence by elongation of a capture nucleotide sequence with detectable modified nucleotides in order to detect the given spots, where the target has been bound with the last nucleotide of the capture nucleotide sequence being complementary of a target sequence at this particular position. The document WO 98/28438 proposes to complete several cycles of hybridization-elongation steps to label a spot in order to compensate for a low hybridization yield of the target sequence. This method allows identification of a nucleotide at a given location of a sequence by labeling of a spot of the elongated capture nucleotide sequence.

Prior to elongation, the capture nucleotide sequences present on the array can be digested by a nuclease in order to differentiate between matched and the unmatched heteroduplexes (U.S. Pat. No. 5,753,439). Use of nuclease for identification of sequences has also been proposed (EP 0721016). A second labeled nucleotide sequence complementary of the targets has also been proposed to be added to the hybridized targets and being ligate to the capture nucleotide sequence if the last nucleotide of the targets is complementary to the targets a this position (WO 96/31622).

The document EP-0785280 proposes a detection of polymorphism based on the hybridization of the target nucleotides on blocks containing several oligonucleotide sequences differing by one base each and obtain a ratio of intensity for determining which sequences are the perfect hybridization matches.

Using membranes or nylon supports are proposed to increase the sensitivity of the detection on solid support by incorporation of a spacer between the support and the capture nucleotide sequences. Van Ness et al. (1991 Nucleic Acids Res. 19:3345) describe a poly(ethyleneimine) arm for the binding of DNA on nylon membranes. The European patent application EP-0511559 describes a hexaethylene glycol derivative as spacer for the binding of small oligonucleotides upon a membrane. When membranes like nylon are used as support, there is no control of the site of binding between the solid support and the oligonucleotides and it was observed that a poly dT tail increased the fixation yield and so the resulting hybridization (WO089/11548). Similar results are obtained with repeated capture sequences present in a polymer (U.S. Pat. No. 5,683,872).

Guo et al. (1994 Nucleic Acids Res. 22:5456) teach the use of poly dT of 15 bases as spacer for the binding of oligonucleotides on glass with increased sensitivity of hybridization.

The document WO99/16780 describes the detection of 4 homologous sequences of the gene femA on nylon strips. However, no data on the sensitivity of the method and the detection is presented. In said document, the capture nucleotide sequences comprise between 15 and 350 bases with homology less than 50% with a consensus sequence.

The publication of Anthony et al. (J. Clin. Microbiol. 38:7817-8820) describes the use of a membrane array for the discrimination with low sensitivity of homologous sequences originated from a several related organisms. Targets to detect are rDNA amplified from bacteria by consensus PCR and the detection is obtained on nylon array containing capture nucleotide sequences for said bacteria and having the capture nucleotide sequences having between 20 and 30 bases which are covalently linked to the nylon, and there is no control of the portion of the sequence which is available for hybridization.

However these patents neither described nor suggested that it is was possible to use a component of a (micro)organism, especially a genetic sequence, to identify said (micro)organism together with the identification of the group to which these (micro)organisms belong. Also there is neither an indication nor a suggestion in the state of the art that polynucleotides can be used as capture sequences in microarrays in order to differentiate a binding between homologous polynucleotides sequences and to permit identification of one target sequence among other species, genus or families of (micro)organisms sequences.

Also there is no indication or suggestion that homologous sequences differing by one nucleotide at one location of the sequence (such as observed in polymorphism analysis) could be detected by hybridization of the amplified sequences on corresponding capture nucleotide sequences.

Prior to the invention, it was unknown that it is possible to identify in a two-step process, i.e. an amplification followed by a direct hybridization of the amplicons on an array, organisms belonging to the same group, to two groups or more together with the specific identification of the groups as such. Also it was unknown that it was possible to identify organisms belonging to a group and sub-group together with the specific identification of these group and sub-group. Also that such identification could be obtained by using polynucleotide as capture sequences for all detections.

Also it was unknown that polynucleotides could be used for the identification of homologous polynucleotide sequences differing by one nucleotide present in a particular location of the sequence.

Also it was unknown that homologous polynucleotide sequences could be discriminated and detected on an array directly after amplification with a very high sensitivity.

The development of the biochips technology allows the detection of multiple nucleotide sequences simultaneously in a given assay and thus allows the identification of the corresponding organism or part of the organism. Arrays are solid supports containing on their surface a series of discrete regions bearing capture nucleotide sequences (or probes) that are able to bind (by hybridisation) to a corresponding target nucleotide sequence(s) possibly present in a sample to be analysed. The present invention enables the detection of the full length double stranded amplicons produced by PCR on the capture probes fixed on a support like the array. If the target sequence is labelled with modified nucleotides during a reverse transcription or an amplification of said sequence, then a signal can be detected and measured at the binding location. Its intensity gives an estimation of the amount of target sequences present in the sample. Such technology allows the identification and/or quantification of genes or species for diagnostic or screening purposes. More particularly, the present invention extends the specific amplification-detection processes of multiple nucleotide sequences even to non homologous sequences.

The present invention provides a new method and device to improve microarrays or biochips technology for the easy identification (detection and/or quantification) of a large number of (micro)organisms or portions of (micro)organisms like their gene transcripts having very different nucleotide sequences. The method is well suited for miniaturized assays where a large amount of information has to be tested or obtained on a small amount of biological material.

Typical applications fitting with these needs are identifications of organisms like bacteria or other pathogenic organisms among many possible other ones which can be responsible for a disease. The present invention may be used for the determination of the presence of SNPs in a genome in order to detect possible genetic diseases. The present invention may also be used for expression analysis on clinical samples where sometimes a few milligrams or even a few micrograms of tissue are available.

The present invention further provides a method and device for getting specific and sensitive detection even for assays suitable for multiple targets. The method is made simple both for the specific amplification of multiple nucleotide sequences even if non homologous by providing derivative nucleic acids which are all amplified by a single primer pair and identifying (detection and/or quantification) the amplified sequences by their direct hybridization on specific capture molecules immobilized in specific locations. Preferably, the invention allows identification and/or recording of single signals upon said locations. The method is particularly suitable when the sample contains nucleotide sequences to be detected at very different concentrations and/or when genomic DNA is present.

The method may be used in diagnostic procedures which employ a closed system containing all reagents for performing this amplification method and which employ a single amplification reaction of all the sequences present in the sample.

The method is also suited for an identification of the genome of pathogenic organisms. It is also useful for quantification of gene expression in cells or tissues, even in degraded form. The method is compatible with detection of amplified target sequences in real time PCR and on microarrays.

### SUMMARY OF THE INVENTION

The present invention is premised in part on the discovery that arrays can be used to obtain a discrimination between a homologous (biological) component (such as a genetic sequence) of different (micro)organisms belonging to several groups together with the identification of these groups as such.

The present invention is especially useful in using arrays to discriminate between homologous genetic sequences (amino acid sequences and nucleotide sequences) belonging to several groups of organisms together with the identification of these groups as such.

The invention provides a method and a device which are based upon a simplified technology requiring the use of a single or limited number of primer pair(s) in an amplification step to detect the presence of the specific target or group of target sequence(s) and followed by the identification (detection and/or quantification) of said specific target or groups of target genetic sequence(s) by recording in a single spot identification upon said micro-array and in the same experimental protocol, said signal being either specific of the organism or the group or sub-group of organisms.

The present invention further provides means for an identification of organisms differing by single base difference of a given nucleotide sequence followed by hybridization of their amplified polynucleotide sequences upon arrays.

### BRIEF DESCRIPTION OF THE DRAWINGS

FIG. 1 is a schematic presentation of the step used in the method of the invention for the identification of 5 Staphylococcus species on biochips after PCR amplification with consensus primers.

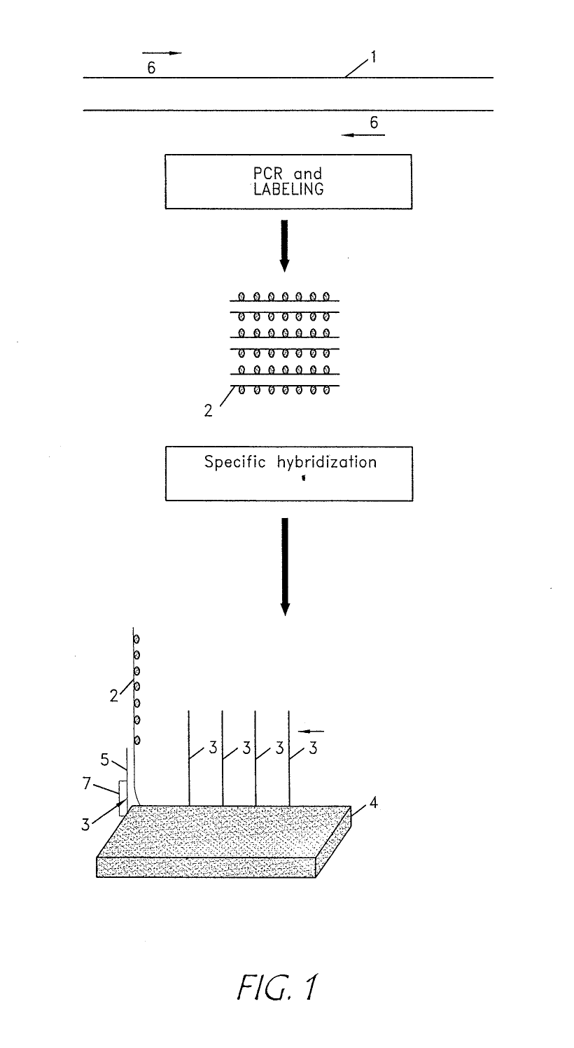

FIG. 2 represents the design of an array which allows the determination of the 5 most common Staphylococcus species, of the presence of any Staphylococcus strain and of the MecA gene.

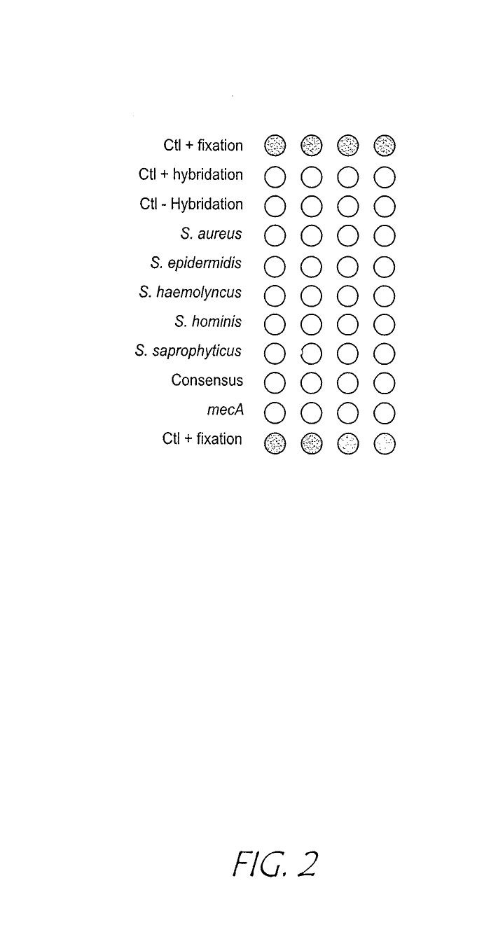

FIG. 3 presents the effect of the length of the specific sequence of a capture nucleotide sequence on the discrimination between sequences with different level of homology.

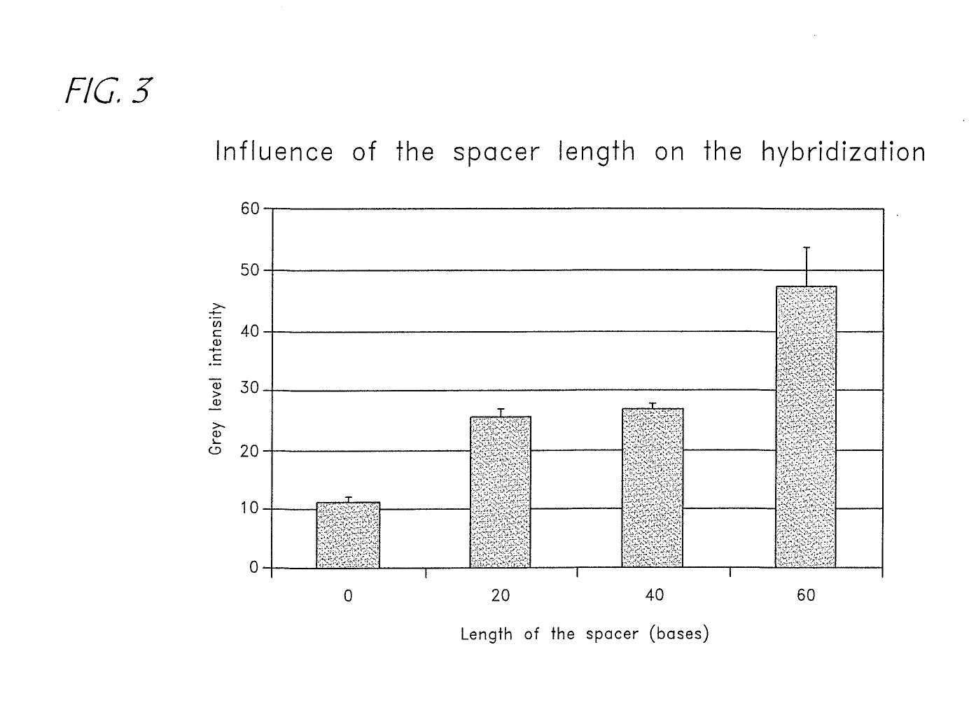

FIG. 4 shows the sensitivity obtained for the detection of FemA sequences from S. aureus on array bearing the small specific capture nucleotide sequence for a S. aureus and a consensus sequence.

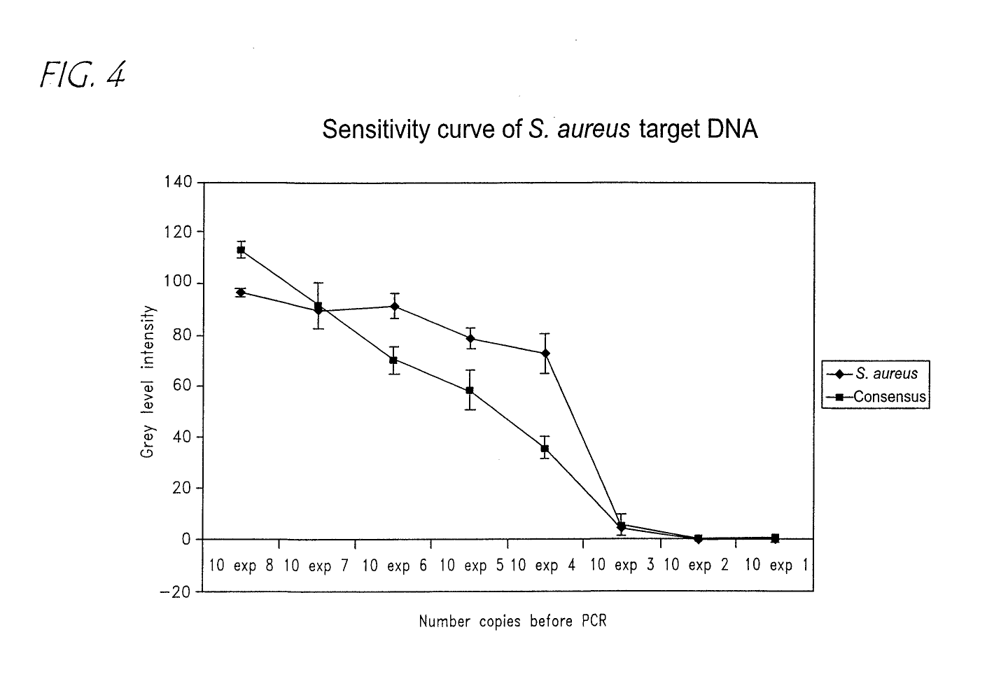

FIG. 5 is a schematic presentation of the steps used in the method of the invention for the identification of different sequences in a sample after obtaining derivatives of the sequences and their amplification by a universal primer pair followed by their hybridization on immobilized specific capture molecules. The figure presents a schematic process for two nucleotide sequences Sa and Sg present in the sample. They are amplified with a primer pair specific for each target sequences (SPa1, SPa2 and SPg1, SPg2) that have an universal amplifying sequences (U). The same amplification solution also contains universal primers (U) having a sequence identical to U and which also participate in the amplification reaction. The amplified target sequences will then hybridize on their specific capture molecules (Ca and Cg).

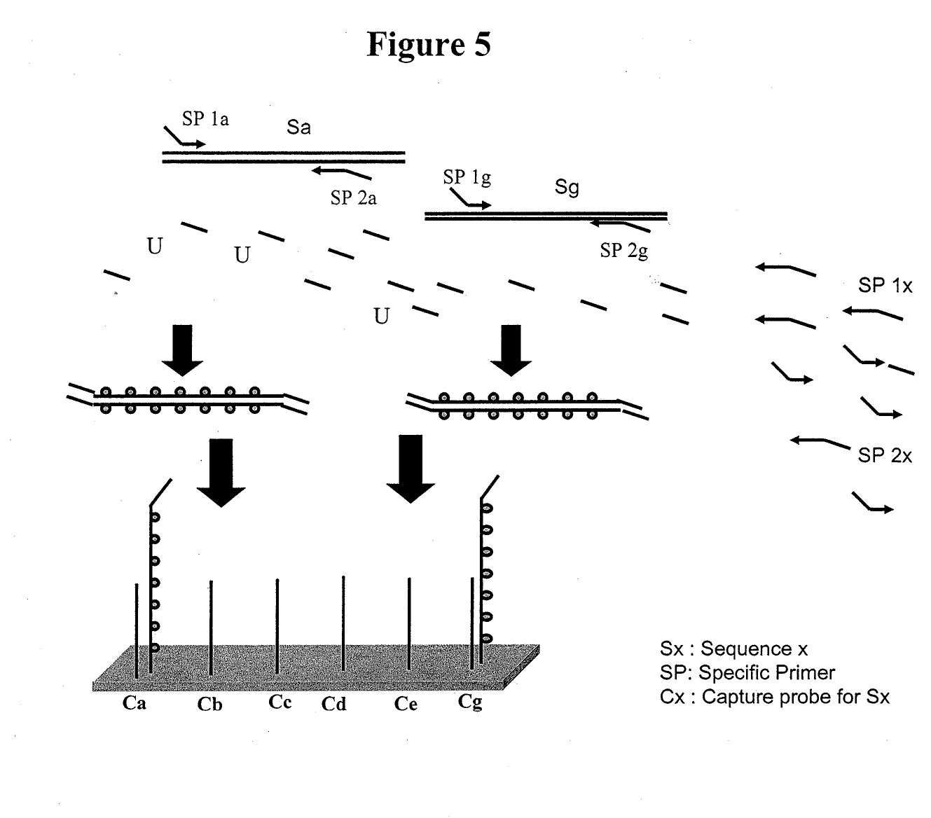

### DETAILED DESCRIPTION OF THE PREFERRED EMBODIMENT

### Definitions

The terms “nucleic acid”, “oligonucleotide”, “array”, “nucleotide sequence”, “target nucleic acid”, “bind substantially”, “hybridizing specifically to”, “background”, “quantifying” are the ones described in the international patent application WO 97/27317 incorporated herein by reference. The term polynucleotide refers to nucleotide or nucleotide like sequences of more than 100 bases long.

The terms “nucleotide triphosphate”, “nucleotide”, “primer sequence” are those described in the document WO 00/72018 and WO 01/31055 incorporated herein by references.

The terms “homologous genetic sequences” mean amino acid or nucleotide sequences having a percentage of amino acids or nucleotides identical at corresponding positions which is higher than in purely random alignments. They are considered as homologous when they show a minimum of homology (or sequence identity) defined as the percentage of identical nucleotides or amino acids found at each position compared to a total of nucleotides or amino acids, after the sequences have been optimally aligned taking into account additions or deletions (like gaps) in one of the two sequences to be compared. Genes coding for a given protein but present in genetically different sources like different organisms are usually homologous. Also in a given organism, genes coding for proteins or enzymes of the same family (Interleukins, Cytochrome b, Cytochrome P450). The degree of homology (or sequence identity) can vary a lot as homologous sequences may be homologous only in one part, a few parts or portions or all along their sequences. The parts or portions of the sequences that are identical in both sequences are said conserved. Protein domains which present a conserved three dimensional structure are usually coded by homologous sequences and even often by a unique exon. The sequences showing a high degree of invariance in their sequences are said to be highly conserved and they present a high degree of homology.

The terms “group, sub-group and sub-sub-group” refer first to the classification of biological organisms in taxas kingdom, branches, classes, orders, families, genus, species, sub-species, varieties or individuals. These constitute different levels of biological taxonomical organization. Groups also refer to organisms which have some aspects in common, but some genetic differences like, for example, the GMO plants, transgenic or chimeric animals. For the purpose of this invention, the common aspects have to be reflected into common or homology DNA or RNA sequences and the dissimilarities or differences in DNA sequences. Gene sequences can also be classified in groups and sub-group independently of their organism origins and are as such part of the invention. They will then refer to groups or sub-groups of genes which belong to a given family such as the cytochrome P450 genes, the protein kinases, the G receptor coupled proteins and others. These genes are homologous to each other as defined here above.

Classification of genes (nucleotide sequences) are used as the basis of molecules paleontology for establishing the classification of organisms into species, genus, family, orders, classes branches, kingdom and taxus.

The terms “hybridization” or “annealing” refer to the formation of duplex DNA strands by nucleotide base pairing. Hybridization yield and specificity is strongly dependant on the incubation conditions especially the temperature and the solution stringency. Conditions have to be worked out in order to optimize the hybridization yield of the specific strands and to minimize the hybridization of unrelated sequences. Stability of the duplex is estimated by the melting temperature (Tm) which represents the temperature for which 50% of the strands will dissociate in given conditions. Determination of the duplex stability can be performed empirically by those skilled in the art considering the variables such as but not limited to the length of the duplex, base composition, ionic strength, and number and position of the mismatches. The Tm will also strongly depend on solution composition, on the ionic strength and on the pH. Tm for perfectly matched small sequences of around 20 bp such as primers can be estimated in reference conditions in a first approximation by the available software methods such as the Primer express or Oligo 6.

Reaction conditions have to be adjusted in order to obtain stringent hybridization conditions in which the complementary sequences will fully or nearly fully hybridize. Such conditions are presented for example in Sambrook et al. (1985 Molecular Cloning—A Laboratory Manual, Cold Spring Harbor Laboratory, Cold Spring Harbor, N.Y.) presented here as reference. Typical stringent solutions used in the PCR are in the range of 0.1 M salt concentration at pH 8. The working conditions are typically chosen in order to be around 5° C. lower than the Tm of the primers and is then adjusted if necessary taking into account the possible presence of mismatches.

The term “homologous sequences” mean nucleotide sequences having a percentage of nucleotides identical at corresponding positions which is higher than in purely random alignments. They are considered as homologous when they show a minimum of homology (or sequence identity) defined as the percentage of identical nucleotides found at each position compared to the total nucleotides, after the sequences have been optimally aligned taking into account additions or deletions (like gaps) in one of the two sequences to be compared. Genes coding for a given protein but present in genetically different sources like different organisms are usually homologous. Also in a given organism, genes coding for proteins or enzymes of the same family (Interleukins, Cytochrome b, P450) are homologous. The degree of homology (or sequence identity) can vary a lot as homologous sequences may be homologous only in one part, a few parts or portions or all along their sequences. The parts or portions of the sequences that are identical in both sequences are said to be conserved. They show identity of sequences. The overall different sequences which include such identical portions of sequences are said to be homologous since some portions of their sequences show a perfect alignment. In some embodiments, the homologous sequences have at least 50% and better at least 70 and even 90 percent nucleotide identity.

Methods of alignment of sequences are based on local homology algorithms which have been computerised and are available as for example (but not limited to) Clustal®, (Intelligenetics, Mountain Views, Calif.), or GAP®, BESTFIT®, FASTA® and TFASTA® (Wisconsin Genetics Software Package, Genetics Computer Group Madison, Wis., USA) or Boxshade®.

The term “consensus sequence” is a sequence determined after alignment of the several homologous sequences to be considered (calculated as the base which is the most commonly found in each position in the compared, aligned, homologous sequences).

The consensus sequence represents a sort of “average” sequence which is as close as possible from all the compared sequences. For high homologous sequences or if the consensus sequence is long enough and the reaction conditions are not too stringent, it can bind to all the homologous sequences. This is especially useful for the amplification of homologous sequences with the same primers called, consensus primers. Experimentally, the consensus sequence calculated from the programs above can be adapted in order to obtain such property.

The terms “primer”, “universal primer” or “specific primer”, “amplification reaction mixture”, “thermostable polymerase” “volume exclusion agent” as mainly used here are defined in the EP141113 (cited above).

The term “primer” refers to an oligonucleotide, whether natural or synthetic, capable of acting as a point of initiation of DNA synthesis under conditions in which synthesis of a primer extension product complementary to a nucleic acid strand is induced, i.e., in the presence of four different nucleoside triphosphates and an agent for polymerization (i.e., DNA polymerase or reverse transcriptase) in an appropriate buffer and at a suitable temperature. Oligonucleotide analogues, such as “peptide nucleic acids”, can act as primers and are encompassed within the meaning of the term “primer” as used herein. A primer is preferably a single-stranded oligodeoxyribonucleotide. The appropriate length of a primer depends on the intended use of the primer but typically ranges from 6 to 50 nucleotides. Short primer molecules generally require cooler temperatures to form sufficiently stable hybrid complexes with the template. A primer need not reflect the exact sequence of the template but must be sufficiently complementary to hybridize with a template. The primers are specific to given sequences or to a family or sequence related polynucleotide and are then considered as consensus for these related sequences.

The PCR reagents described herein are provided and used in PCR in suitable concentrations to provide amplification of the target nucleic acid. The minimal amount of DNA polymerase is generally at least about 1 unit/100 μl of solution, with from about 4 to about 25 units/100 μl being preferred. A “unit” is defined herein as the amount of enzyme activity required to incorporate 10 nmoles of total nucleotides (dNTPs) into an extending nucleic acid chain in 30 minutes at 74° C. The concentration of each primer is at least about 0.025 μmolar and less than about 1 μmolar with from about 0.05 to about 0.2 μmolar being preferred. All primers are present in about the same amount (within a variation of 10% of each). The cofactor is generally present in an amount of from about 1 to about 15 μmolar, and each dNTP is generally present at from about 0.15 to about 3.5 mmolar in the reaction mixture. The volume exclusion agent is present in an amount of at least about 1 weight percent, with amounts within the range of from about 1 to about 20 weight % being preferred. As used in defining the amounts of materials, the term “about” refers to a variation of +/−10% of the indicated amount.

An “amplification reaction mixture”, which refers to a solution containing reagents necessary to carry out an amplification reaction refers, as used herein, to an aqueous solution comprising the various amplification reagents used to amplify a target nucleic acid. The reagents include primers, enzymes, aqueous buffers, salts, target nucleic acid, and deoxynucleoside triphosphates (both conventional and unconventional). Depending on the context, the mixture can be either a complete or incomplete reaction mixture. A “PCR reaction mixture” typically contains oligonucleotide primers, a thermostable DNA polymerase, dNTPs, and a divalent metal cation in a suitable buffer.

A reaction mixture is referred to as complete if it contains all reagents necessary to enable the reaction, and incomplete if it contains only a subset of the necessary reagents. It will be understood by those skilled in the art that reaction components are routinely stored as separate solutions, each containing a subset of the total components, for reasons of convenience, storage stability, and to allow for independent adjustment of the concentrations of the components depending on the application, and, furthermore, that reaction components are combined prior to the reaction to create a complete reaction mixture.

The term “thermostable DNA polymerase” refers to an enzyme that is relatively stable to heat and catalyzes the polymerization of nucleoside triphosphates to form primer extension products that are complementary to one of the nucleic acid strands of the target sequence. The enzyme initiates synthesis at the 3′ end of the primer and proceeds in the direction toward the 5′ end of the template until synthesis terminates. Purified thermostable DNA polymerases can be selected from the genera Thermus, Pyrococcus Thermococcus and Thermotoga, preferably Thermus aquaticus, Pyrococcus furiosus, Pyrococcus woesei, Pyrococcus spec. (strain KOD1), Pyrococcus spec. GB-D, Thermococcus Litoralis Thermococcus sp. 9° N-7, Thermotoga maritima, Pyrococcus spec. ES4 (endeavori), Pyrococcus spec. OT3 (horikoshii), Pyrococcus profundus, Thermococcus stetteri, Thermococcus spec. AN1 (zilligii), Thermococcus peptonophilus, Thermococus celer and Thermococcus fumicolans.

The term “thermostable enzyme” refers to an enzyme that is relatively stable to heat. The thermostable enzymes can withstand the high temperature incubation used to remove the modifier groups, typically greater than 50° C., without suffering an irreversible loss of activity. The hot start DNA polymerases are enzymes or enzyme conditions which make then less active in the original conditions but their activity increased during a first heating at high temperature usually above 90° C.

The term “volume exclusion agent”, as defined herein, refers to one or more water-soluble or water-swellable, nonionic, polymeric volume exclusion agents.

The general principles and conditions for amplification and detection of nucleic acids using polymerase chain reaction are quite well known and are described in numerous references including U.S. Pat. No. 4,683,195, U.S. Pat. No. 4,683,202 and U.S. Pat. No. 4,965,188, incorporated herein by reference. Thus, in view of the teaching in the art and the specific teaching provided herein, a worker skilled in the art should have no difficulty in practicing the present invention by making the adjustments taught herein to co-amplify several nucleic acids, one of which may be a low copy target nucleic acid or may be preferentially amplified.

The term “Real Time PCR” means a method which allows detecting and/or quantifying the presence of the amplicons during the PCR cycles. In the Real Time PCR, the presence of the amplicons is detected and/or quantified in at least one of the cycles of amplification. The increase of amplicons or signal related to the amount of amplicons formed during the PCR cycles is used for the detection and/or quantification of a given nucleotide sequence in the PCR solution.

Micro-arrays are described extensively in EP1266034 and in US20040229225, the disclosures of which are incorporated herein by reference in their entireties. “Micro-array” means a support on which multiple capture molecules are immobilized in order to be able to bind to the given specific target molecule. The micro-array is preferentially composed of capture molecules present at specifically localized areas on the surface or within the support or on the substrate covering the support. A specifically localized area is the area of the surface which contains bound capture molecules specific for a determined target molecule. The specific localized area is either known by the method of building the micro-array or is defined during or after the detection. A spot is the area where specific target molecules are fixed on their capture molecules and seen by the detector. Immobilization of capture molecules on insoluble support is also possible in the form of lines.

The term “organisms” includes live microbial entities as such, such as bacteria or fungi, and comprises parts thereof, the presence of which may be identified with the present method. Hence, in case an organism produces a particular entity, such as a particular protein, the identification of the genetic material of said organism (such as its genomic DNA or its mRNA) allows the determination of whether said part of the organism is present in the sample.

### Part I

The present invention is related to an identification and/or quantification method of a biological (micro)organism or a (biological) component thereof, said (micro)organism or its component being possibly present in a sample, preferably a biological sample, among at least two, preferably at least four, other related (micro)organisms or components; said method comprising the step of:
possibly extracting original components from the (micro)organisms;
possibly labeling said (micro)organism or its components being target;
putting into contact the (micro)organism or its components being targets with capture molecules bound to an insoluble support, preferably a non-porous solid support; and
discriminating the binding of said targets, specific of a (micro)organism or its component by detecting, quantifying and/or recording a signal resulting from the specific binding between said targets and their corresponding specific capture molecules; wherein said capture molecules are bound to an insoluble solid support at a specific location according to an array, said array having a density of at least 4 different bound capture molecules/cm² of solid support surface and wherein the binding between the targets and their corresponding capture molecules forms said signal at the expected location, the detection of a single signal allowing a discrimination of a target being specific of said (micro)organism or its components from other related (micro)organisms or other related components.

Advantageously, said method further comprises the step of identifying and/or quantifying the presence of several groups, subgroups or sub-subgroups of components or (micro)organisms, comprising said components being related to each other until possible individual genetic sequences (nucleotide and/or amino acid sequences) wherein the binding of targets and corresponding specific capture molecules forms a signal at an expected location allowing the identification of a target specific of a group, sub-group or sub-subgroup of components or (micro)organisms comprising said components.

Therefore, the biological component according to the invention could be a nucleotide sequence specific of a (micro)organism or an amino acid sequence (peptide) specific of a (micro)organism. Examples of said molecules are homologous nucleotide sequences or peptides presenting a high homology such as receptors, HLA molecules, cytochrome P450, etc.

Furthermore, the inventors have discovered that it is possible to drastically simplify the identification or quantification of one or several (micro)organisms among many other ones present in such biological sample, said identification and/or quantification being obtained by combining a single amplification using common primer pairs and an identification of the possible (micro)organisms by detecting, quantifying and/or possibly recording upon an array the presence of a single signal resulting only between a capture nucleotide sequence and its corresponding target nucleotide sequence and thereafter correlating the presence of said detected target nucleotide sequence to the identification of a nucleotide sequence specific of said (micro)organism(s).

This means that the method and device according to the invention will allow the easy identification/detection of a specific sequence among other homologous sequences and possibly its quantification (characterization of the number of copies or presence of said organisms in a biological sample) of a target nucleotide sequence, said target sequence having a nucleotide sequence specific of said (micro)organisms.

Such identification may be obtained directly, after washing of possible contaminants (unbound sequences), by detecting and possibly recording a single spot signal at one specific location, wherein said capture nucleotide sequence was previously bound and said identification is not a result of an analysis of a specific pattern upon the microarray as proposed in the system of the state of the art. Therefore, said method and device do not necessarily need a detailed analysis of said pattern by an image processing and a software analysis.

This invention was made possible by discovering that target sequences can be discriminated from other homologous ones upon an array with high sensitivity by using bound capture nucleotide sequences composed of at least two parts, one being a spacer bound by a single and advantageously predetermined (defined) link to the support (preferably a non porous support) and the other part being a specific nucleotide sequence able to hybridize with the nucleotide target sequence.

Furthermore, said detection is greatly increased, if high concentrations of capture nucleotide sequences are bound to the surface of the solid support.

The present invention is related to the identification of a target nucleotide sequence obtained from a biological (micro)organism or a portion thereof, especially a gene possibly present in a biological sample from at least 4 other homologous (micro)organisms or a portion thereof, said other (micro)organisms could be present in the same biological sample and have homologous nucleotide sequences with the target.

Said identification is obtained firstly by a genetic amplification of said nucleotide sequences (target and homologous sequences) by common primer pairs followed (after washing) by discrimination between the possible different target amplified nucleotide sequences. Said discrimination is advantageously obtained by hybridization upon the surface of an array containing capture nucleotide sequences at a given location, specific for a target nucleotide sequence specific for each (micro)organism to be possibly present in the biological sample and by the identification of said specific target nucleotide sequence through the identification and possibly the recording of a signal resulting from the specific binding of this target nucleotide sequence upon its corresponding capture nucleotide sequence at the expected location (single location signal being specific).

According to the invention, the preferred method for genetic amplification is the PCR using two anti-parallel consensus primers which can recognize all said target homologous nucleotide sequences but other genetic amplification methods may be used.

Therefore, said (micro)organisms could be present in any biological material or sample including genetic material obtained (virus, fungi, bacteria, plant or animal cell, including the human body). The biological sample can be also any culture medium wherein microorganisms, xenobiotics or pollutants are present, as well as such extract obtained from a plant or an animal (including a human) organ, tissue, cell or biological fluid (blood, serum, urine, sputum, etc).

The method according to the invention can be performed by using a specific identification (diagnostic and/or quantification) kit or device comprising at least an insoluble solid support upon which are bound single stranded capture nucleotide sequences (preferably bound to the surface of the solid support by a direct covalent link or by the intermediate of a spacer) according to an array with a density of at least 4, preferably at least 10, 16, 20, 50, 100, 1000, 4000, 10 000 or more, different single stranded capture nucleotide sequences/cm² insoluble solid support surface, said single stranded capture nucleotide sequences having advantageously a length comprised between about 30 and about 600 bases (including the spacer) and containing a sequence of about 3 to about 60 bases, said sequence being specific for the target (which means that said bases of said sequence are able to form a binding with their complementary bases upon the sequence of the target by complementary hybridization). Preferably, said hybridization is obtained under stringent conditions (under conditions well-known to the person skilled in the art).

In the method and kit or device according to the invention, the capture nucleotide sequence is a sequence having between 16 and 600 bases, preferably between 30 and 300 bases, more preferably between 40 and 150 bases and the spacer is a chemical chain of at least 6.8 nm long (of at least 4 carbon chains), a nucleotide sequence of more than 15 bases or is nucleotide derivative such as PMA.

The method, kit and device according to the invention are particularly suitable for the identification of a target, being preferably biological (micro)organisms or a part of it, possibly present in a biological sample where at least 4, 12, 15 or even more homologous sequences are present. Because of the high homology, said nucleotide sequence can be amplified by common primer(s) so that the identification of the target nucleotide sequence is obtained specifically by the discrimination following its binding with the corresponding capture nucleotide sequence, previously bound at a given location upon the microarray. The sensitivity can be also greater increased if capture nucleotide sequences are spotted to the solid support surface by a robot at high density according to an array. A preferred embodiment of the invention is to use an amount of capture nucleotide sequences spotted on the array resulting in the binding of between about 0.01 to about 5 pmoles of sequence equivalent/cm² of solid support surface.

The kit or device according to the invention may also incorporate various media or devices for performing the method according to the invention. Said kit (or device) can also be included in an automatic apparatus such as a high throughput screening apparatus for the detection and/or the quantification of multiple nucleotide sequences present in a biological sample to be analyzed. Said kit or apparatus can be adapted for performing all the steps or only several specific steps of the method according to the invention.

In the method, the kit (device) or apparatus according to the invention, the length of the bound capture nucleotide sequences is preferably comprised between about 30 and about 600 bases, preferably between about 40 and about 400 bases and more preferably between about 40 and about 150 bases. Longer nucleotide sequences can be used if they do not lower the binding yield of the target nucleotide sequences usually by adopting hairpin based secondary structure or by interaction with each other.

In a preferred embodiment, the specific part of the capture nucleotide sequence is bound onto a nucleotide sequence of between 20 and 600 bases.

In another preferred embodiment, all capture molecules are polynucleotides of more than 100 base long.

In another embodiment, the capture nucleotide sequence is linked to a polymer molecule bound to the solid support. The polymer is preferably a chain of at least 10 atoms, selected from the group consisting of poly-ethyleneglycol, polyaminoacids, polyacrylamide, poly-aminosaccharides, polyglucides, polyamides, polyacrylate, polycarbonate, polyepoxides or poly-ester (possibly branched polymers).

If the homology between the sequences to be detected is low (between 30 and 60%), parts of the sequence which are specific in each sequence can be used for the design of specific capture nucleotide sequences binding each of the different target sequences. However, it is more difficult to find part of the sequence sufficiently conserved as to design “consensus” sequences which will amplify or copy all desired sequences. If one pair of consensus primers is not enough to amplify all the homologous sequences, then a mixture of two or more primers pairs is added in order to obtain the desired amplifications. The minimum homologous sequences amplified by the same consensus primer is two, nut there is no limitation to said number.

If the sequences show high degree of homology, higher than 60% and even higher than 90%, then the finding of common sequence for consensus primer is easily obtained, but the choice for specific capture nucleotide sequences become more difficult.

In another preferred embodiment of the invention, the capture nucleotide sequences are chemically synthesized oligonucleotides sequences shorter than 100 bases (easily performed on programmed automatic synthesizer). Such sequences can bear a functionalized group for covalent attachment upon the support, at high concentrations.

Longer capture nucleotide sequences are preferably synthesized by (PCR) amplification (of a sequence incorporated into a plasmid containing the specific part of the capture nucleotide sequence and the non specific part (spacer)).

In a further embodiment of the invention, the specific sequence of the capture nucleotide sequence is separated from the surface of the solid support by at least about 6.8 nm long, equivalent to the distance of at least 20 base pair long nucleotides in double helix form.

In the method, kit (device) or apparatus according to the invention, the portion(s) (or part(ies)) of the capture nucleotide sequences complementary to the target is comprised between about 3 and about 60 bases, preferably between about 15 and about 40 bases and more preferably between about 20 and about 30 bases. These bases are preferably assigned as a continuous sequence located at or near the extremity of the capture nucleotide sequence. This sequence is considered as the specific sequence for the detection. In a preferred form of the invention, the sequence located between the specific capture nucleotide sequence and the support is a non specific sequence.

In another embodiment of the invention, a specific nucleotide sequence comprising between about 3 and about 60 bases, preferably between about 15 and about 40 bases and more preferably between about 20 and about 30 bases is located on a capture nucleotide sequence comprising a sequence between about 30 and about 600 bases.

The method, kit (device) or apparatus according to the invention are suitable for the detection and/or the quantification of a target which is made of DNA or RNA, including sequences which are partially or totally homologous upon their total length.

The method according to the invention can be performed even when a target present between an homology (or sequence identity) greater than 30%, greater than 60% and even greater than 80% and other molecules.

In the method, kit (device) or apparatus according to the invention, the capture nucleotide sequences are advantageously covalently bound (or fixed) upon the insoluble solid support, preferably by one of their extremities as described hereafter.

The method according to the invention gives significant results which allows identification (detection and quantification) with amplicons in solutions at concentration of lower than about 10 nM, of lower than about 1 nM, preferably of lower than about 0.1 nM and more preferably of lower than about 0.01 nM (=1 fmole/100 μl).

Another important aspect of this invention is to use very concentrate capture nucleotide sequences on the surface. If too low, the yield of the binding is quickly lower and is undetectable. Concentrations of capture nucleotide sequences between about 600 and about 3,000 nM in the spotting solutions are preferred. However, concentrations as low as about 100 nM still give positive results in favorable cases (when the yield of covalent fixation is high or when the target to be detected is single stranded and present in high concentrations). Such low spotting concentrations would give density of capture nucleotide sequence as low as 20 fmoles per cm². On the other side, higher density was only limited in the assays by the concentrations of the capture solutions, but concentrations still higher than 3,000 nM give good results.

The use of these very high concentrations and long nucleotide sequences are two unexpected characteristic features of the invention. The theory of DNA hybridization proposed that the rate of hybridization between two DNA complementary sequences in solution is proportional to the square root of the DNA length, the smaller one being the limited factor (Wetmur, J. G. and Davidson, N. 1968 J. Mol. Biol. 3:584). In order to obtain the required specificity, the specific sequences of the capture nucleotide sequences had to be small compared to the target. Moreover, the targets were obtained after PCR amplification and were double stranded so that they reassociate in solution much faster than to hybridize on small sequences fixed on a solid support where diffusion is low thus reducing even more the rate of reaction. It was unexpected to observe a so large increase in the yield of hybridization with the same short specific sequence.

The amount of a target which “binds” on the spots is small compared to the amount of capture nucleotide sequences present. So there is a large excess of capture nucleotide sequence and there was no increase of binding if more capture nucleotide sequences were present.

One may perform the detection on the full length sequence obtained after amplification or copy and when labeling is performed by incorporation of labeled nucleotides, more markers are present on the hybridized target making the assay sensitive.

The method, kit and apparatus according to the invention may comprise the use of other bound capture nucleotide sequences, which may have the same characteristics as the previous ones and may be used to identifying a target from another group of homologous sequences (preferably amplified by common primer(s)).

In the microbiological field, one may use consensus primer(s) specific for each family, or genus, of micro-organisms and then identify some or all the species of these various family in an array by using capture nucleotide sequences of the invention. Detection of other sequences can be advantageously performed on the same array (i.e. by allowing an hybridization with a standard nucleotide sequence used for the quantification, with consensus capture nucleotide sequences for the same or different micro-organisms strains, with a sequence allowing a detection of a possible antibiotic resistance gene by micro-organisms or for positive or negative control of hybridization). Said other capture nucleotide sequences have (possibly) a specific sequence longer than 10 to 60 bases and a total length as high as 600 bases and are also bound upon the insoluble solid support (preferably in the array made with the other bound capture nucleotide sequences related to the invention). A long capture nucleotide sequence may also be present on the array as consensus capture nucleotide sequence for hybridization with all sequences of the microorganisms from the same family or genus, thus giving the information on the presence or not of a microorganism of such family, genus in the biological sample.

The same array can also bear capture nucleotide sequences specific for a bacterial group and as specific application to Gram-positive or Gram-negative strains or even all the bacteria.

Another application is the detection of homologous genes from a consensus protein of the same species, such as various cytochromes P450 by specific capture nucleotide sequences with or without the presence of a consensus capture nucleotide sequence for all the cytochromes P450 possibly present in a biological sample. Such detection is performed at the gene level by reverse transcription into cDNA.

The solid support according to the invention can be or can be made with materials selected from the group consisting of glasses, electronic devices, silicon supports, plastic supports, silica, metal or a mixture thereof in format such as slides, compact discs, gel layers, microbeads. Advantageously, said solid support is a single glass slide which may comprise additional means (barcodes, markers, etc.) or media for improving the method according to the invention.

The amplification step used in the method according to the invention is advantageously obtained by well known amplification protocols, preferably selected from the group consisting of PCR, RT-PCR, LCR, CPT, NASBA, ICR or Avalanche DNA techniques.

Advantageously, the target nucleotide sequence to be identified is labeled previously to its hybridization with the single stranded capture nucleotide sequences. Said labeling (with known techniques from the person skilled in the art) is preferably also obtained upon the amplified sequence previously to the denaturation (if the method includes an amplification step).

Advantageously, the length of the target nucleotide sequence is selected as being of a limited length preferably between 50 and 2000 bases, preferably between 100 and 400 bases and more preferably between 100 and 200 bases. This preferred requirement depends on the possibility to find consensus primers to amplify the required sequences possibly present in the sample. Too long target nucleotide sequence may reallocate faster and adopt secondary structures which can inhibit the fixation on the capture nucleotide sequences.

The amplified target nucleotide sequence can be cut before the hybridization, and the use of one capture sequence for each target sequence to make the interpretation of the results easy.

The detection of homologous expressed genes is obtained by first reverse transcription of the mRNA by a consensus primer, the preferred one being the poly dT. In one embodiment, the reverse transcribed cDNA is then amplified by consensus primers as described in this invention.

According to a further aspect of the present invention, the method, kit (device) or apparatus according to the invention is advantageously used for the identification of different Staphylococcus species or variant, preferably the S. aureus, the S. epidermidis, the S. saprophyticus, the S. hominis or the S. haemolyticus for homologous organs present together or separately in the biological sample, said identification being obtained by detecting the genetic variants of the FemA gene in said different species, preferably by using a common locations in the FemA genetic sequence (examples 4, 5, 6, 7). In another aspect of the invention, 16 Staphylococcus species could be detected after amplification by the same primers and identification on the array (example 7).

Preferably, the primer(s) and the specific portions of said FemA sequence used for obtaining amplified products are the ones described hereafter in Example 2. These primers have been selected as consensus primers for the amplification of the FemA genes of all of the 16 Staphylococcus tested and they probably will amplify the FemA from all other possible Staphylococcus species.

A further aspect of the invention is the detection of Mycobacteria species, the M. tuberculosis and other species, preferably the M. avium, M. gastrii, M. gordonae, M. intracellulare, M. leprae, M. kansasi, M. malmoense, M. marinum, M. scrofulaceum, M. simiae, M. szulgai, M. xenopi, M. ulcerans (Example 8).

In a further application of the invention, one array can specifically detect amplified sequences from several bacterial species belonging to the same genus (Examples 7 and 8) or from several genus like Staphylococcus, Streptococcus, Enterococcus, Haemophilus (see Table 1) or different bacterial species and genus belonging to the Gram-positive bacteria and/or to the Gram-negative bacteria (Examples 16 and 22).

Preferably, the primer(s) and the specific portions of gyrase (sub-unit A) sequences are used for obtaining amplified products. These primers have been selected as consensus primers for the amplification of the gyrase genes of all of the bacteria tested and they probably will amplify the gyrase from many other possible bacteria species and genus and families.

The invention is particularly suitable for detection of bacteria belonging to at least two of the following genus families: Staphylococcus, Enterococcus, Streptococcus, Haemolyticus, Pseudomonas, Campylobacter, Enterobacter, Neisseria, Proteus, Salmonella, Simonsiella, Riemerella, Escherichia, Neisseria, Meningococcus, Moraxella, Kingella, Chromobacterium, Branhamella.

The array allows to read the MAGE number by observation of the lines positive for signal bearing the specific capture nucleotide sequences.

The same application was developed for the G Protein Coupled Receptors (GPCR). These receptors bind all sorts of ligands and are responsible for the signal transduction to the cytoplasm and very often to the nucleus by modulating the activity of the transcriptional factors. Consensus primers are formed for the various subtypes of GPCR for dopamine and for serotonin and histamine. The same is possible for the histamine and other ligands.

The detection of the various HLA types is also one of the applications of the invention. HLA are homologous sequences which differ from one individual to the other. The determination of the HLA type is especially useful in tissue transplantation in order to determine the degree of compatibility between the donor and the recipient. It is also a useful parameter for immunization. Given the large number of subtypes and the close relation between the homologous sequences it was not always possible to perfectly discriminate one sequence among all the other ones and for some of them there was one or two cross-reactions. In this case, a second capture nucleotide sequence complementary to another location of the amplified sequence was added on the array, in order to make the identification absolute.

Genetic sequences code for proteins so that homologous DNA sequences correspond to homologous amino acid sequences of the encoded proteins while variation in the DNA sequences correspond to variation in amino acid sequence. One embodiment of this invention is to use antibodies for specific capture of proteins from a sample in order to identify the protein and so the organism from which it originates. By choosing appropriate antibodies, the organisms or the group to which it belongs is determined. The HLA typing is given as example of the use of specific antibodies for discriminating the various HLA-A proteins on an array (Example 23).

Discrimination of the Cytochrome P450 forms is one particular application of the invention (Example 14).

Detection of polymorphism sequences (which can be considered as homologous even if differing by only one base) can be made also by the method according to the invention. This is especially useful for the Cytochrome P450 since the presence of certain isoforms modifies the metabolism of some drugs. The invention was found particularly useful for discriminating between the isoforms of Cyto P450 2D6 and 2C19. More generally the invention is particularly well adapted for the discrimination of sequences differing by one base mutation or deletion called Single Nucleotide Polymorphism (SNP). The originality of the invention is to perform the hybridization step directly on the amplified sequences without the necessity to copy into RNA and to cut them into pieces.

Furthermore, one array can specifically detect amplified sequences from several animal species and genus belonging to several families like Galinacea, Leporidae, Suidae and Bovidae (Table 2).

One array can specifically detect amplified sequences from several fishes species, such as G. morhua, G. macrocephalus, P. flesus, M merluccius, O. mykiss, P. platessa, P. virens, S. salar, S. pilchardus, A. thazard, T. alalunga, T. obesus, R. hippoglossoides, S. trutta, S. sarda, T. thynnus, S. scombrus belonging to several genera such as Auxis, Sarda, Scomber, Thunnus, Oncorhynch, Salmo, Merluccius, Pleuronectes, Platichtlys, Reinhardtius, Pollachius, Gadus, Sardina, from several families such as Scombridae, Salmonidae, Merluccidae, Pleuronectidae, Gadidae and Clupeidae (Table 3). Other homologous sequences allow the determination of plant species and genuses such as Potato, tomato, oryza, zea, soja, wheat, barley, bean, carrot belonging to several families (Example 19).

According to a further aspect of the present invention, the method, kit (device) or apparatus according to the invention is advantageously used for the identification of the origin of meat (Table 2).

Preferably, the primer(s) and the specific portions of cytochrome b sequences are used for obtaining amplified products are the ones described hereafter in Example 3. These primers have been selected as consensus primers for the amplification of the cytochrome B genes of all of animals tested and they probably will amplify the cytochrome B from many other animals species, genus and families.

According to a further aspect of the present invention, the method, kit (device) or apparatus according to the invention is advantageously used for the identification of the origin of fishes (table 3).

Preferably, the primer(s) and the specific portions of said cytochrome b sequences used for obtaining amplified products are the ones described hereafter in Example 18. These primers have been selected as consensus primers for the amplification of the cytochrome B genes of all of fishes tested and they probably will amplify the cytochrome B from many other fish species, genuses and families.

According to a further aspect of the present invention, the method, kit (device) or apparatus according to the invention is advantageously used for the identification of the origin of plants.

Preferably, the primer(s) and the specific portions of said sucrose synthase sequences used for obtaining amplified products are the ones described hereafter in the examples. These primers have been selected as consensus primers for the amplification of the sucrose synthase genes of all of plants tested and they probably will amplify the sucrose synthase from many other plants species, genus and families.

According to a further aspect of the present invention, the method, kit (device) or apparatus according to the invention is advantageously used for the identification of the Genetically Modified Organism (GMO). The GMO are produced by insertion into the genome of an organism of one or several external genes together with other regulating or construction sequences.

Preferably, the primer(s) and the specific portions of said sucrose synthase sequences used for obtaining amplified products are the ones described hereafter in the examples. These primers have been selected as consensus primers.

Homologous DNA or RNA sequences lead to the expression in cells or tissues of proteins which are also homologous to each other. Therefore, a target component to be detected may be protein which is related to other homologous ones which could be present in the same biological sample. Related proteins means proteins which have some part(s) of their sequence or conformation in common, while said proteins present other part(s) which are specific or the (micro)organisms or a part of said (micro)organisms from which they originate.

Part or portion of the amino acid sequences are identical between proteins from the same group while other portions are specific of the target to be identified and possibly quantified. Said amino acid sequences present linear or conformational epitopes which can be recognized by specific (monoclonal) antibodies. The discrimination between said specific related targets is possible by specific antibodies or reconstructed antibodies like proteins bearing hypervariable portions of these antibodies. An identification of said common homologous sequences is also possible by using antibodies directed against the common sequence. Therefore, discrimination between groups, subgroups, sub-subgroups and individual proteins can be made in a single experiment.

Preferably, antibodies are bound to the solid support as array and are used for the specific capture of the target's components to be identified. For HLA identification, proteins are classified in class I, II and III antigens. The class I is divided into the HLA-A, B, C, E, F and G. Each of them being subdivided into HLA types and subtypes as given in the databank IMGT/HLA. There are more than 476 different alleles of the class I HLA antigens. The heavy chains of the HLA complex of type I possess regions as the α1 and α2 domains which are very polymorphic while other parts as the α3 is more conserved (Auffray and Strominger, 1986 Advanced Hum. Genet. 15:197). The class II is divided into the HLA-DR, HLA-DP and HLA-DQ. There are more than 430 alleles of the HLA class II. Each type is subdivided into subtypes and sub-subtypes which can be discriminated according to the present invention (Example 23).

In one of the aspects of the invention, typing of Cytochrome P450 proteins is performed using the antibodies directed against cytochrome P450 1A1, 1A2, 2A6, 2C11, 3A4, 4A. These antibodies are available from ABR (Golden, Colo., USA).

According to a further aspect of the present invention, the method, kit (device) or apparatus according to the invention is advantageously used for the identification of the organisms or part of it as provided in the examples cited here above and also the ones presented in the Examples 1 to 23.

Another aspect of the present invention is related to any part of biochips or microarray comprising said above described sequences (especially the specific capture nucleotide sequence described in the examples) as well as a general screening method for the identification of a target sequence specific of said microorganisms of family type discriminated from homologous sequences upon any type of microarrays or biochips by any method.

After hybridization on the array, the target sequences can be detected by current techniques. Without labeling, preferred methods are the identification of the target by mass spectrometry now adapted to the arrays (U.S. Pat. No. 5,821,060) or by intercalating agents followed by fluorescent detection(WO 97/27329).

The labeled associated detections are numerous. A review of the different labeling molecules is given in WO97/27317. They are obtained using either already labeled primer or by incorporation of labeled nucleotides during the copy or amplification step. A labeling can also be obtained by ligating a detectable moiety onto the RNA or DNA to be tested (a labeled oligonucleotide, which is ligated, at the end of the sequence by a ligase). Fragments of RNA or DNA can also incorporate labeled nucleotides at their 5′-OH or 3′-OH ends using a kinase, a transferase or a similar enzyme.

The most frequently used labels are fluorochromes like Cy3, Cy5 and Cy7 suitable for analyzing an array by using commercially available array scanners (General Scanning, Genetic Microsystem). Radioactive labeling, cold labeling or indirect labeling with small molecules recognized thereafter by specific ligands (streptavidin or antibodies) are common methods. The resulting signal of target fixation on the array is either fluorescent, colorimetric, diffusion, electroluminescent, bio- or chemiluminescent, magnetic, electric like impedometric or voltammetric (U.S. Pat. No. 5,312,527). A preferred method is based upon the use of the gold labeling of the bound target in order to obtain a precipitate or silver staining which is then easily detected and quantified by a scanner.

Quantification has to take into account not only the hybridization yield and detection scale on the array (which is identical for target and reference sequences) but also the extraction, the amplification (or copying) and the labeling steps.

The method according to the invention may also comprise means for obtaining a quantification of target nucleotide sequences by using a standard nucleotide sequence (external or internal standard) added at known concentration. A capture nucleotide sequence is also present on the array so as to fix the standard in the same conditions as said target (possibly after amplification or copying); the method comprising the step of quantification of a signal resulting from the formation of a double stranded nucleotide sequence formed by complementary base pairing between the capture nucleotide sequences and the standard and the step of a correlation analysis of signal resulting from the formation of said double stranded nucleotide sequence with the signal resulting from the double stranded nucleotide sequence formed by complementary base pairing between capture nucleotide sequence(s) and the target in order to quantify the presence of the original nucleotide sequence to be detected and/or quantified in the biological sample.

Advantageously the standard is added in the initial biological sample or after the extraction step and is amplified or copied with the same primers and/or has a length and a GC content identical or differing from no more than 20% to the target. More preferably, the standard can be designed as a competitive internal standard having the characteristics of the internal standard found in the document WO 98/11253. Said internal standard has a part of its sequence common to the target and a specific part which is different. It also has at or near its two ends sequences which are complementary of the two primers used for amplification or copy of the target and similar GC content (WO 98/11253). In the preferred embodiment of this invention, the common part of the standard and the target, means a nucleotide sequence which is homologous to all target amplified by the same primers (i.e. which belong to the same family or organisms to be quantified).

Preferably, the hybridization yield of the standard through this specific sequence is identical or differ no more than 20% from the hybridization yield of the target sequence and quantification is obtained as described in WO 98/11253.

Said standard nucleotide sequence, external and/or internal standard, is also advantageously included in the kit (device) or apparatus according to the invention, possibly with all the media and means necessary for performing the different steps according to the invention (hybridization and culture media, polymerase and other enzymes, standard sequence(s), labeling molecule(s), etc.).

Advantageously, the solid support of the biochips also contain spots with various concentrations (i.e. 4) of labeled capture nucleotide sequences. These labeled capture nucleotide sequences are spotted from known concentrations solutions and their signals allow the conversion of the results of hybridization into absolute amounts. They also allow to test for the reproducibility of the detection.

The solid support of the biochips can be inserted in a support connected to another chamber and automatic machine through the control of liquid solution based upon the use of microfluidic technology. By being inserted into such a microlaboratory system, it can be incubated, heated, washed and labeled by automates, even for preliminary steps (like extraction of DNA, genetic amplification steps) or the identification and discrimination steps (labeling and detection). All these steps can be performed upon the same solid support.

The present invention is also related to a method to identify homologous sequences (and the groups to which they belong and eventually the organisms and their groups) possibly present in a biological sample by assay of their genetic material in a array-type format. The method is well adapted for determination of organisms belonging to several groups being themselves members of a super-group. The method is for example well adapted for a biological determination and/or classification of animals, plants, fungi or micro-organisms.

The method involves the use of multiple capture nucleotide sequences present as arrays, the capture of the corresponding target sequences and their analysis and possibly their quantification. The method also allows the identification of these organisms and their groups by characterization of the positive area of the arrays bearing the required capture nucleotide sequences. One particular specification of the invention being that a positive hybridization resulting in one spot on the array, gives the necessary information for the identification of the sequence or the organism or the group or sub-group from which it belongs by the person skilled in the art.

It also provides a method for sequential analysis of the presence of any researched organisms during the genetic amplification followed by the detection of amplicons on the array and identification of the corresponding organisms or groups thereafter.

Furthermore, the inventors have discovered that is possible to obtain by the method of the invention a very quick and easy identification of such multiple sequences belonging to several groups or sub-groups or sub-sub-groups of sequences being homologous to each others, until possible individual sequences, by combining a single nucleotide amplification, preferably by PCR, using common primer pair(s) together with an identification of the organisms at different level(s) by detecting and possibly recording upon an array having at least 5 different bound single stranded capture nucleotide sequences/cm² of solid support surface, the presence of a single signal resulting from the binding between a capture sequence and its (or their) corresponding target sequence(s) and thereafter correlating the presence of said detected target sequences to the identification of a specific genetic sequence among the other ones. The method is especially well adapted for the identification of organism species, genus and family through the analysis of a given part of their genome or gene expressed, these sequences being homologous to each other in the different organisms.

A single signal means a signal which by itself is sufficient to identify one or more target nucleotide sequence(s) to which it is designed and therefore to give (if necessary) an unambiguous response for the presence or not of the organisms or groups of organism present in the sample or the organisms or group of organisms from which said sample has been obtained.

The method and device according to the invention allows easy identification/detection of a specific nucleotide sequences among other possible amplified nucleotide sequences and possibly their quantification (characterization of the number of copies or presence of said organisms in a biological sample) of target sequences, said target nucleotide sequences having a nucleotide sequence specific of said organisms or groups of organisms.

The array may contain capture nucleotide sequences from several organism genus and from several of these genus species. The capture nucleotide sequences may detect the genus, the species and also the family(ies) to which these genus belong. The capture nucleotide sequences may also detect the sub-species and even the individual organisms of one or several species. Individual organisms of a given species are considered as having very homologous sequences differing mainly by single bases within some of their DNA sequences or genes. Homology is important for getting consensus primers and a single base change is sufficient to obtain discrimination between two target amplicons. If not completed, the discrimination can be confirmed by the use of second capture nucleotide sequences present upon the array and able to bind a same amplicon at different sequence location.

Said identification is obtained firstly by a genetic amplification of said nucleotide sequences (target sequences) by common primer pair followed (after washing) by discrimination between the possible different targets amplified according to the above described method.

The amplified sequences may belong to the same gene, may be part of the same DNA locus and are homologous to each others.

The method according to the invention further comprises the step of correlating the signal of detection (possibly recorded) to the presence of:

specific organism(s) groups,

specific organism(s) sub-groups until the possible individuals,

genetic characteristics of a sequence from a organism,

polymorphism of said sequence,

genotyping of organisms based on differences in DNA or RNA sequences,

diagnostic predisposition or evolution (monitoring) of genetic diseases, including cancer of a patient (including the human) from which the biological sample has been obtained.

The method also applies to the identification and possibly characterization of nucleotide sequences as such independently of the organism. Genes or DNA sequences can be classified in groups and sub-groups and sub-sub-groups according to their sequence homology. Bioinformatic programs exist for sequence alignment and comparison (such as Clustal, Intelligenetics, Mountain View, Calif., or GAP, BESTFIT, FASTA and TFASTA in the Wisconsin Genetics Software Package, Genetics computer Group Madison, Wis., USA or Boxshade). A classification can be made according to the percentage of homology and alignment of the sequences. An interest in detection and identification of the sequences from a given family in a given organism, tissue or cell is for example the possibility to detect the effect of any given molecules, biological or pathological conditions (by proteomics, functional genomics, etc.) upon both the overall and the specific genes of one or several families.

The inventors also found that sensitivity of the assay was increased by using high density of capture nucleotide sequences fixed on the support, being preferably higher than about 100 fmoles/cm² of solid support surface.

The capture nucleotide sequences specific for the determination of a group of organisms are designed in a way as to be able to specifically capture the different sequences belonging to the various groups. These capture nucleotide sequences are called consensus for this group of organisms. The consensus capture nucleotide sequences may contain specific sequences which are longer than the specific capture nucleotide sequences of the different members of the group. These capture nucleotide sequences are consensus sequences, (i.e. the sequences containing at each of its location the base which is the most present in the different sequences of the members of the group when aligned). In another embodiment the consensus capture nucleotide sequence has the length of the amplified sequences.

The inventors have found unexpected results in that the same identification of several organisms of several groups can be performed at the organisms as well as at the level in the same experimental conditions. Identification of the groups required long capture nucleotide sequences while the specific identification of the organism require small, but specific capture sequences. The inventors found that using the characteristic of the invention, mainly by binding of the specific part of the sequences onto a spacer, it was possible to obtain both results in the same experimental conditions. The invention allows also using the same stringency conditions, meanly determined by the salt concentration and the temperature and the rate of reaction.

According to the invention, organisms are identified as such by their specific polymorphism. Single base substitution in a particular location of genome is the characteristic of an individual organism among others of the same species. The method for identification of the polymorphism is part of the invention with direct hybridization of the amplified sequences on the capture nucleotide sequences of the array and detection of the fixed target sequence.

The detection of the target sequence being bound on capture nucleotide sequences is obtained through the labeling of the capture nucleotide sequence on which the target sequence is bound. A step of capture nucleotide sequences labeling is added after the hybridization step. The extension of the capture nucleotide sequence free end, preferably the 3′ end) is performed using detectable nucleotide, preferably a biotin or fluorescent nucleotide, and a polymerization agent, preferably a DNA polymerase and the necessary reagent for making the extension. The target sequence hybridized on the capture nucleotide sequence serves as matrix for the extension; the hybridized target sequences are then removed from the capture nucleotide sequence, rehybridized and extension of the capture nucleotide sequence performed.

The invention allows identification of the presence of a polymorphism by using an array having at least five different bounded single stranded capture polynucleotide sequence/cm² of solid support surface, the determination of a single signal resulting from the binding between the capture sequence and the target sequence, extending at least one polynucleotide primer of the hybrid beyond the 3′ terminal nucleotide thereof in the 3′ to 5′ direction using the polynucleotide sequence as a template, said extension is effected in the presence of polymerization agent and nucleotide precursor wherein at least one nucleotide incorporated into the extended primer molecule is a detectably-modified nucleotide; denaturing the duplex to free the target sequence from the polynucleotide capture nucleotide sequence, carry out step one or more times and detecting the presence of a signal associated with the detectable modified nucleotide in the extended capture nucleotide sequence at the reaction zone to effect said determination.

The process is repeated as needed to obtain a signal detectable on the array. A preferred signal is obtained in colorimetry using the silver precipitation as proposed and detection of the array on colorimetric detector (WO 00/72018). The arrays may be present in the surface of multiwells and multiwell plate detectors used for the reading of the results.

In another embodiment, a second labeled nucleotide sequence complementary to the target sequence and adjacent to the capture nucleotide sequence is added on the hybridized amplicons and a ligation performed. If the last base of the capture nucleotide sequence is complementary to the target sequence, then ligation will occur and the spot is labeled. If not ligation will not occur even if the target amplicon is hybridized on the capture nucleotide sequence.

In a particular embodiment the array bear in separated area several identical capture nucleotide sequences differing only by one nucleotide located at the same place in the capture nucleotide sequence, the last free end is the interrogation base. The array is then able to identify the presence of any of the 4 bases present at a given location of the sequence. Such array is especially useful when detecting polymorphism in homozygote or heterozygote organism or when the polymorphism is not known.

In the method, kit (device) or apparatus according to the invention, the portion(s) (or part(ies)) of the capture nucleotide sequences complementary to the target sequence is composed of at least two families. The first one comprised between about 5 and about 60 bases, preferably between about 15 and about 40 bases and more preferably between about 20 and about 30 bases. In the second capture family, the binding parts of the capture nucleotide sequence sequences are comprised between about 10 and 1000 bases and preferably between 100 and 600 bases. These bases are preferably assigned as a continuous sequence located at or near the extremity of the capture nucleotide sequence. This sequence is considered as the specific sequence for the detection. In a preferred form of the invention, the sequence located between the specific capture nucleotide sequence and the support surface is a non-specific sequence.

In another preferred embodiment of the invention, the first family of capture nucleotide sequences detect the members of a group while the second family of capture nucleotide sequences detect the group as such.

However, both families of capture nucleotide sequences can be polynucleotides.

All the capture sequences present on the array necessary for capturing the target sequences are polynucleotides and are able to detect both the members of a group and the groups or sub-groups themselves.

The consensus primers can be chosen in order to amplify different sequences and groups of sequences.

The same pair of primers amplifies several groups of sequences being different for the different groups of homologous sequences, each one being associated with one or several group of organism.

The pair of consensus primers may be associated with group identification and/or for species identification on the array.

A second or third (or even more) primers are added for the amplification step in order to possibly amplify other sequences, related or not to one particular group and useful to be detected in the sample. Virus susceptible to be present in a clinical sample together with bacteria is one of the examples where such extension of the invention is particularly useful like the combination of virus detection of example 17 with bacteria detection of examples 7, 8 or 16.

Two pairs of (possibly consensus) primers may be used for the amplification, (one for amplification of sequences of the gram-positive and the other one for the gram-negative bacteria, the amplified sequences are specific of each of the gram-positive or the gram-negative bacteria and detected thereafter on the array as specific bacteria species or/and genus and/or family).

Each of the two primers pair amplifies various sequences specific of one or several families which are then detected as specific species or/and genus, families on the array.

The same array can also bear capture nucleotides sequences specific for bacterial families or genus.

In one preferred embodiment of the invention, the detection of the presence of any member of the groups are first detected during the PCR using method like the real time PCR and the amplicons are thereafter used for identification on the array.

Real time PCR is performed in specific machines which along the PCR cycle detect the appearance of fluorescence in the solution. Increase in fluorescence is due to the insertion of fluorochromes such as in the double stranded amplicons produced during the PCR cycles.

Specific fluorescent labeled nucleotide sequences are added to the PCR solution for specific identification of the amplicons. These nucleotide sequences are complementary to the amplified target sequences and their fluorescence emission is limited by the presence at the right position of a scavenger. Once digested by the polymerase during the copying of the amplicons, the fluorochrome is released in solution where it is detected. Said method is called Fluorescence Resonance Emission Transfert (FRET. The sequence is chosen so as to bind to a consensus region of the detected amplicons or several nucleotide sequences are chosen in consensus regions specific of the groups of sequences or organisms to be detected. These nucleotide sequences are preferably labeled with different fluorochromes so as to identify the group during the amplification step.

The fluorescent signal of the amplification solution is registered and if crossing a threshold, the solution is processed for hybridization on capture nucleotide sequences of the array. In a preferred embodiment a solid support bearing the array is added in the amplification chamber and in the hybridization processes. In another preferred embodiment the hybridization is performed on the surface of the same chamber as the PCR. Chambers, preferably closed chambers, can be of any size, format and material as compatible with arrays as already mentioned here above. The chambers may be in polymers such as polycarbonate, polypropylene, or glass such as capillaries. Polyacrylate based surfaces are particularly useful since they are transparent to light and allow covalent binding of capture probes necessary for the arrays. The free end, of the capture nucleotide sequence can be either a 5′ or 3′ —OH or phosphate group modified in order to avoid elongation. Preferably, the specific sequence portion of the capture nucleotide sequence has a melting temperature smaller than the primers used for the amplification in order to avoid hybridization during the PCR cycles. Also the hybridization may be performed at a given temperature using the heating and control system of the amplification cycler. A control process provides on the amplification cycler to continue or not the detection on the array after the amplification steps.

The real time PCR may be performed with the primers amplifying the gram-positive or/and the gram-negative PCR and thereafter the families or/and the genes or/and the species identified on the array.

One embodiment of the invention is to combine in one process the real time PCR together with the hybridization on capture probes for identification of the target molecules or organisms. In a preferred embodiment the process is performed in the same chamber and with the same machine device.

The present invention also covers the machine and apparatus necessary for performing the various steps of the process mainly for diagnostic and/or quantification of a (micro)organism or component possibly present in a sample among at least two, preferably at least 4 other related (micro)organisms which comprises:

capture molecules being bound to an insoluble solid support at specific locations according to an array, said capture molecules being able to discriminate between related (micro)organisms or components, said array having a density of at least 4 discrete regions per cm² solid support surface;

a detection and/or quantification device of a signal formed at the location of the binding between said target compound with said capture molecule;

possibly reading device of information recorded upon said solid support;

a computer program to recognize the discrete regions bearing the target molecules and their locations; and

correlating the presence of the signal at these locations with the detection and/or quantification of the said (micro)organism or component.

In a particular embodiment, this apparatus also performs the genetic amplification of the nucleotide sequences by PCR performed previously or in real time together with the identification of a (micro)organism or its components.

Detection of other sequences can be advantageously performed on the same array (i.e. by allowing an hybridization with a standard nucleotide sequence used for the quantification, with consensus capture nucleotide sequences for the same or different micro-organisms strains, with a sequence allowing a detection of a possible antibiotic resistance gene by micro-organisms or for positive or negative control of hybridization). Said other capture nucleotide sequences have (possibly) a specific sequence longer than 10 to 60 bases and a total length as high as 600 bases and are also bound upon the insoluble solid support (preferably in the array made with the other bound capture nucleotide sequences related to the invention).

These characteristics described in details for a specific detection and analysis of nucleotide sequences can be adapted by the person skilled in the art for other components of (micro)organisms such as receptors, antibodies, enzymes, etc.

The present invention will be described in details in the following non-limiting examples in reference to the enclosed figures and tables.

### BRIEF DESCRIPTION OF THE TABLES

Table 1 presents identification of 3 gram-positive and 1 gram-negative bacteria at the genus level (horizontally) and at the species level (vertically). These bacteria are detected with the method of the invention on biochips after PCR amplification with consensus primers. The PCR was realized on the gyrase (sub-unit A) sequences.

TABLE 1

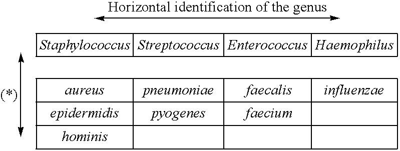

(*) Identification of the species

Table 2: The identification of meat animals at the family level (horizontally) and at the genus and species levels (vertically) (3 levels of classification), detected with the method of the invention on biochips after PCR amplification with consensus primers. The PCR was realized on Cytochrome B gene sequences.

TABLE 2
Meat

| Galinacea | Leporidae | Suidae | Bovidae |
|---|---|---|---|
| Chicken | Rabbit | Pig | Cow |
| Duck | | Wild | Brownswiss, Jersey, Hereford, |
| | | pig | Simmental, Piemontaise, Canadienne, |
| | | | RedAngus, Limousine, AberdeenAngus, |
| | | | Butana, Charolais, Fresian, Kenana, |
| | | | N'Dama |
| Ostrich | | | |
| Turkey | | | |
| Quail | | | |

Table 3 presents the identification of fishes at the family level (horizontally) and at the genus and species levels (vertically) (3 levels of classification), detected with the method of the invention on biochips after PCR amplification with consensus primers. The PCR was realized on Cytochrome B gene sequences.

### Part II

The inventors have also discovered that it is possible to drastically simplify the identification of one or several (micro)organisms among many other ones having different sequences and being present in a sample even at different concentrations by combining a single amplification using a common primer pair together with amplification with primers specific of the different nucleotide sequences. In some embodiments, the invention involves detecting and possibly recording the presence of a single signal resulting only from a binding between an immobilized capture sequence and its corresponding target sequence and correlating the presence of said detected target sequence to the identification of a genetic sequence specific of said (micro) organism(s). The method and device according to the invention allow the easy identification/detection of a specific sequence among other sequences and possibly its quantification (characterization of the number of copies or presence of said organisms in a biological sample) of a target sequence, said target sequence having a nucleotide sequence specific of said (micro) organisms.

The present invention provides a method for identifying and/or quantifying an organism or part of an organism in a sample by detecting a nucleotide sequence specific of said organism, among at least 4 other nucleotide sequences from other organisms or from parts of the organism comprising the steps of:
producing derived sequences from the said organism nucleotide sequences by incorporation of at least one common sequence in said organism nucleotide sequences in order to obtain a partial homology between the said derived nucleotide sequences
amplifying said derived nucleotide sequences by PCR into double stranded target nucleotide sequences using a unique pair of primers, which recognize the common sequence of the derived sequences and which are capable of amplifying at least 4 of said other derived nucleotide sequences as to produce full-length target nucleotide sequences having between 60 and 800 bases;
contacting said full-length target nucleotide sequences resulting from the amplifying step with at least 5 different single-stranded capture nucleotide sequences having between about 55 and about 800 bases, preferably between about 60 and about 450 bases, said single-stranded capture nucleotide sequences being covalently bound in an microarray to insoluble solid support(s) and wherein said capture nucleotide sequences comprising a nucleotide sequence of at least 15 bases which is able to specifically bind to said full-length target nucleotide sequence without binding to said at least 4 other derived nucleotide sequences, and said specific sequence is separated from the surface of the solid support by a spacer comprising a nucleotide sequence of at least 40 bases in length; and
detecting specific hybridization of said target nucleotide sequence to said capture nucleotide sequences present at specific locations.

In some embodiments, the identification is performed directly or after washing off possible contaminants (unbound sequences), by detecting and possibly recording a single spot signal at one specific location, wherein said capture nucleotide sequence was previously bound and said identification is the result of said signal at the expected location and is not a result of an analysis of a specific pattern upon a microarray as proposed in the system of the state of the art. Therefore, said method and device do not necessarily need a detailed analysis of said pattern by an image processing and a software analysis.

This invention was made possible by discovering that target sequences can be discriminated from other ones upon an array with high sensitivity by using bound capture nucleotide sequences composed of at least two parts, one being a spacer bound by a single and advantageously predetermined (defined) link to the support (preferably a non porous support) and the other part being a specific nucleotide sequence able to hybridize with the nucleotide target sequence. The target molecule binds to its specific complementary sequence of the probe and this sequence is separated from the solid support surface by nucleotides acting as a spacer. Such configuration of the capture molecules leads to a high hybridization yield and/or to a stabilization of the target sequences which makes possible the detection of full length molecules even in the presence of their complementary sequences present in the same hybridization solution. This effect is reproducible and valid for different target molecule to be detected. This result also allows hybridization of the full length amplified sequences without them being further cut into pieces or without them being transformed into single strand sequences, which was unexpected given the constraints of the hybridization on solid support.

Furthermore, said detection is greatly increased, if high concentrations of capture nucleotide sequences are bound to the surface of the solid support.

In some embodiments, the present invention is related to the identification of a target sequence obtained from a biological (micro)organism or a portion thereof. For example, the target gene may be present in a biological sample which contains at least 4 other (micro)organisms or portions thereof. Such a method is also well applicable to detection of the components or portions of an organism such as different gene transcripts.

In some embodiments, said identification is obtained firstly by a genetic amplification of said nucleotide sequences (target and homologous sequences) by a common primer pair together with primers specific for the nucleotide sequences followed (after washing if necessary) by a discrimination between the possible different targets amplified. In some embodiments, said discrimination is advantageously obtained by hybridization upon a surface containing capture nucleotide sequences at a given location, specific for a target specific for each (micro)organism which may be possibly present in the biological sample and by the identification of said specific target through the identification and possibly the recording of a signal resulting from the specific binding of this target upon its corresponding capture sequence at the expected location (single location signal being specific for the target).

This embodiment of the invention is related to an unexpected improvement of multiplex amplification methods, preferably a PCR amplification, working in tandem with the detection on immobilized capture molecules allowing analysis of at least 5, 10, 20, 40 different polynucleotide target sequences being possibly present at different concentrations in a given sample. In the amplification step, this embodiment of the invention prevents the use of high concentrations of primers specific for each of the nucleotides to be detected as in a normal PCR. This embodiment of the invention opens the way for the detection of unrelated sequences and is useful in many biological applications such as pathogen detection or the identification of transcripts or of different polymorphisms.

According to this embodiment of the invention, the preferred method for genetic amplification is the PCR. Each nucleotide sequence to be detected is amplified by the combination of two primer pairs; the first one being specific for the nucleotide sequence and leading to the production of derived sequences and the second one being common (universal primer pair) for all the target nucleotides which will be detected and identified thereafter. The derived sequences are best obtained by amplification (or copy) of the nucleotide sequences present in the sample using specific primer pairs, each member of said primer pair comprises a sequence complementary to one of the two strands of a given polynucleotide sequence and a common sequence (U) serving as an universal amplifying sequence, being identical for all the specific primers. In some embodiments, U is at least 15, and preferably at least 20 nucleotides, in length and is located at the 5′ end of the primers. Preferably, U does not contain more than 10 and, more preferably no more than 5, successive bases complementary to the target polynucleotides. During the first amplification cycles, the common sequence (U) will be incorporated into the amplicons and it will then serve in the later cycles as an universal amplifying sequence. Its complementary sequence will then be recognized by the unique primer pair which in this particular embodiment is composed of a single primer having a sequence identical to the common sequence of the specific primers. “Identical sequences” means that they share at least 10 and, more preferably, 15 bases.

In a particular embodiment, the two members of a given primer pair have two different common sequences (U1 and U2) which are used as two universal amplifying sequences. Each specific nucleotide sequence needs two primers to be amplified, each one being complementary of one of the two nucleotide opposite strands. In this particular embodiment, the two primers of a given pair have two different common sequences and the unique primer pair is composed of a pair of sequences having a sequence identical to these two common sequences (U1 and U2). Preferably, the identical sequences share at least 10 and, more preferably, 15 bases.

In another embodiment, the length of the sequence complementary to one of the two strands of a given polynucleotide sequence of the specific primer pair is at least 6 and, more preferably, at least 15 nucleotides. In another embodiment, the sequences complementary to the strands of the polynucleotide sequences of the specific primer pairs show a homology of lower than 50% and preferably lower than 30%.

In the preferred embodiment the nucleotide sequences of the sample to be detected have less than 50% and preferably less that 30% homology to each other. In a particular embodiment, the homology of the amplified target sequences show a low homology being lower than 50% and preferably below 30% so that they are not considered as homologous to each other.

The method according to this embodiment of the invention may further comprise the step of correlating the signal of detection (possibly recorded) to the presence of specific (micro)organism(s), genetic characteristics of a sequence, polymorphism of a sequence, diagnostic predisposition or evolution (monitoring) of genetic diseases, including cancer of a patient (including a human) from which the biological sample has been obtained.

Therefore, said (micro)organisms could be present in any biological material including genetic material obtained. The biological material may comprise virus, fungi, bacteria, plant or animal cells, including biological samples, obtained from humans. The biological sample can be also any culture medium wherein microorganisms, xenobiotics or pollutants are present, as well as such extract obtained from a plant or an animal (including a human) organ, tissue, cell or biological fluid (blood, serum, urine, etc).

The method according to this embodiment of the invention is performed by using a specific identification (diagnostic and/or quantification) kit or device comprising at least an insoluble solid support upon which are bound single stranded capture nucleotide sequences. Preferably the capture molecules are bound to an insoluble solid support (by a direct covalent link or by the intermediate of a spacer) at a specific location according to an array, said array having a density of at least 4, preferably at least 10, 16, 20, 50, 100, 1000, 4000, 10 000 or more, different bound single stranded capture nucleotide sequences/cm² insoluble solid support surface. In another embodiment, the capture probes are bound to different solid support. In some embodiments, the different solid supports are beads, each bead having bound a capture molecule specific for a target so that identification of the location of the binding of a specific capture molecule can be performed.

In some embodiments, the single stranded capture nucleotide sequences have a length comprised between about 30 and about 600 bases (including the spacer) and containing a specific sequence or capture portion (able to hybridize with their corresponding target nucleotide) of at least 15, preferably at least 40, more preferably at least 60 and even more preferably more than 100 continuous nucleotide sequence complementary to one of the two strands of the amplified target sequences, said sequence being specific for the target (which means that said bases of said sequence are able to bind with their complementary bases upon the sequence of the target by complementary hybridization). Preferably, said hybridization is obtained under stringent conditions (under conditions well-known to the person skilled in the art).

Advantageously, when the nucleotide sequence specific for the organism or part of the organism to be identified and/or quantified in a sample is non homologous or the homology is low (less than 30% homology) with other sequences from other organisms possibly present in the same sample, the length of the specific sequence of the capture nucleotide sequence can be increased significantly in order to have a high hybridization yield with the target amplified nucleotide sequences. As the homology between the amplified target nucleotide sequences is low, the risk of cross-hybridization on long capture nucleotide sequence is also low. As a consequence, the specificity of the assay is kept even when long specific sequences are used. In a particular embodiment, the length of the specific sequence of the capture nucleotide sequence is preferably of more than 100 bases, and even more than 200 bases and even more than 400 bases.

The length of the capture molecules is preferably to be limited in order to reduce or avoid cross-reaction with other target sequences. The detection of possible cross-reaction on the capture molecule can be first tested theoretically by comparison of the sequences with the appropriate software as known by the person skilled in the art and/or by experimental assay. Also, long nucleotide sequences can be used if they do not lower the binding yield of the target nucleotide sequences usually by adopting hairpin based secondary structure or by interaction with each other. The length of the target specific sequence of the capture nucleotide sequence is preferably limited to about 600 bases and preferably to about 450 bases and even to about 150 bases.

In the method and kit or device according to this embodiment of the invention, the capture nucleotide sequence is a sequence having between 16 and 600 bases, preferably between 30 and 300 bases, more preferably between 40 and 150 bases and the spacer or spacer portion is a chemical chain of at least 6.8 nm long (corresponding to a nucleotide sequence of 20 bases), comprising a nucleotide sequence of at least 20 bases, better at least 40 bases and even longer than 60 bases or is a nucleotide derivative such as PMA or LNA.

In a preferred embodiment, the nucleotide sequence located between the specific capture nucleotide sequence and the support is a non-specific sequence which is not homologous or identical to the target to be detected. In a particular embodiment, the spacer sequence of a particular capture molecule is a sequence which is complementary to the nucleotide sequences to be detected. It will serve as spacer by separation of the at least 15 bases complementary to the amplified target from the support by at least 20 and better 40 bases.

In a preferred embodiment, the binding of the amplicons on the capture probe is such as to produce two non complementary ends, one being a spacer end and the other one a non-spacer end, such that the spacer end is non-complementary to the spacer portion of the capture molecule and said spacer end exceeds said non-spacer end by at least 50 bases.

In still another preferred embodiment, the detection is performed by hybridization of the full length of amplified sequence upon capture molecules.

In a preferred embodiment, the quantification of the organism present in the biological sample is obtained by the quantification of the signal present at a particular location of the support.

The method, kit and device according to this embodiment of the invention are particularly suitable for the identification of a target, being preferably biological (micro)organisms or a part thereof, possibly present in a biological sample where at least 4, 10, 20 or even more different sequences are possibly present. In some embodiments, said sequences are amplified by specific primers and are made homologous by the incorporation of a common sequence(s) present together on the specific primers and are then amplified by common primers. Because of the presence of common sequences at two different locations of their sequence, the derived sequences are amplified to a detectable amount by a common primer pair. Also given their difference in sequences their identification is obtained by the discrimination following its binding with the corresponding capture nucleotide sequence, previously bound at a given location upon a solid support. The sensitivity can be also greater increased if capture nucleotide sequences are spotted to the solid support surface by a robot at high density according to an array. A preferred embodiment of the invention is to use an amount of capture nucleotide sequences spotted on the array resulting in the binding of between about 0.01 to about 5 pmoles of sequence equivalent/cm² of solid support surface.

The kit or device according to this embodiment of the invention may also incorporate various media or devices for performing the method according to the invention. Said kit (or device) can also be included in an automatic apparatus such as a high throughput screening apparatus for the detection and/or the quantification of multiple nucleotide sequences present in a biological sample to be analyzed. Said kit or apparatus can be adapted for performing all the steps or only several specific steps of the method according to this embodiment of the invention.

In the method, the kit (device) or apparatus according to this embodiment of the invention, the bound single stranded capture nucleotide sequences contain a sequence of between about 10 and about 600 bases, preferably between about 50 and about 450 bases specific for a target nucleotide sequence to be detected and/or quantified and having a total length comprised between about 30 and about 800 bases comprising a spacer having a nucleotide sequence of at least about 20 bases, preferably at least about 40 bases, preferably at least about 60 bases.

It was found that multiple genes or genomic DNA which are unrelated to the other in terms of sequence could be amplified together in one amplification (PCR) solution containing a limited concentration of primers specific of the different nucleotide sequences to be detected so that the total primer concentration in the amplification solution is comprised between 0.5 and 4 μM and better between 0.5 and 2 μM with the achievement thereafter of high yield of the different amplicons with the help of a unique primer pair so that the amplicons are then in sufficient amount to be detected after binding on specific probes.

The method is especially useful when the assay is designed to detect a large number of possible organisms (such as 10 or even 20 or more than 40) so that the amplification solution has to contain specific primers for all these organism nucleotide sequences but the number of actually detected organisms in the sample is limited. This is typically the situation of a diagnostic assay where the number of possible pathogens is large but indeed only one or a few of them are present in a given sample. In this case the amplification method allies both the specificity by the use of specific primer but avoid the problems occurring with the use of high primer concentrations. The use of a common primer pair at high concentration provides finally enough amplicons of any of the present pathogen(s) for their detection on the specific capture probes.

The present amplification method drastically reduces the non-specific amplification due to very low concentrations in the amplification solution of the different target specific primers. This feature is especially useful when working on real biological samples which contain genomic DNA from the host.

In a particular embodiment, the production of the derived sequences from the organism nucleotide sequences of the sample and the amplification by the universal primers are performed in one amplification process.

Preferably the length of the sequence complementary to one of the two strands of a given polynucleotide sequence of the specific primer pair is at least 6 and, more preferably, at least 15 nucleotides. In the preferred embodiment, the primers are designed to be specific for a given nucleotide sequence to be detected. However in a particular application, the primers are random sequences of small sequences of around 6 to 14 bases which are used for the amplification (for example for the transcripts). One of the applications is the amplification of transcripts or random sequences in a genome. The primers can be linked by a ligase to a polynucleotide if needed and processed for further amplification with the universal primers.

In another embodiment, the primers specific for the targets are at a concentration lower than 100 nM in the PCR solution, and may be even lower than 50 nM, or even lower than 20 10 nM.

Also in a preferred embodiment, the concentration of the universal primers is at least 100 nM, and preferably at least 500 nM, and even more preferably at least 1000 nM.

Also in a preferred embodiment, the ratio between the concentration of universal primers and the concentration of the specific target primers in the amplification PCR solution is at least 20:1, more preferably at least 50:1, and even more preferably at least 100:1.

In a preferred embodiment, the total concentration of the overall specific primers does not exceed 2000 nM, and preferably does not exceed 1000 nM, and still more preferably does not exceed 500 nM.

In still another preferred embodiment, the concentration of the overall specific primers does not exceed the concentrations of the universal primers.

Preferably, the universal primers have a Tm ±5° C., and, preferably, ±2° C. of the primers specific for the sample nucleotide sequences.

In still a preferred embodiment, the annealing temperature of the PCR cycles is at least 5° C., and, preferably, at least 7° C. lower than the Tm of the specific and the universal primers.

In particular embodiment, the PCR amplification is obtained with less than 25, and, preferably, less than 20, and even more preferably less 15 cycles and the detection is performed on such amplified sequences.

In particular embodiment, the concentration ratio between 2 different polynucleotide target sequences being detected is higher than 10.

In particular embodiment, the amplification (PCR) solution comprises at least 20 and preferably at least 40 and even more preferably at least 60 different target specific primers.

In a preferred embodiment, the ratio between the concentrations of the two universal primers in the amplification solution is comprised between 1.2 and 2.

In particular embodiment, the amount of non specific amplified sequences represents less than 50% and even less than 20% of the specific amplified sequences.

In particular embodiment, the PCR amplification is performed by a DNA polymerase which is a hot-start DNA polymerase.

In particular embodiment, the PCR amplification is performed by a DNA polymerase which is a Topo Taq DNA polymerase.

The method is not only applicable to amplification and detection of full size genes, but also to degraded genes and is well suited for degraded genes extracted from paraffin embedded tissues, where some chemical modifications of the mRNA occur due to the presence of chemical fixing agents. The present method is fully compatible and well adapted in term of sensitivity and specificity in combination with detection on microarray and also with a real time PCR performed on arrays.

In another preferred embodiment of the invention, the capture nucleotide sequences are chemically synthesized oligonucleotides sequences shorter than 100 bases (easily performed on programmed automatic synthesizer). Such sequences can bear a functionalized group such as amino group for covalent attachment upon the support, at high concentrations.

Longer capture nucleotide sequences are preferably synthesized by PCR amplification (of a sequence incorporated into a plasmid containing the specific part of the capture nucleotide sequence and the non specific part (spacer)).

In a further embodiment of the invention, the specific sequence of the capture nucleotide sequence is separated from the surface of the solid support by a spacer which is at least about 6.8 nm long, equivalent to the distance of at least 20 base pair long nucleotides in double helix form or equivalent to the size of the streptavidin or avidin protein when used as a linker between the capture molecules and the support.

In another embodiment of the invention, a specific nucleotide sequence comprising between about 10 and about 60 bases, preferably between about 15 and about 40 bases and more preferably between about 20 and about 30 bases is located on a capture nucleotide sequence comprising a sequence between about 30 and about 600 bases.

The method, kit (device) or apparatus according to the invention are suitable for the detection and/or the quantification of a target which is made of DNA or RNA, including sequences which are partially or totally homologous upon their total length.

In the method, kit (device) or apparatus according to the invention, the capture nucleotide sequences are advantageously covalently bound (or fixed) upon the insoluble solid support, preferably by one of their extremities as described hereafter.

The method according to the invention gives significant results which allows identification (detection and quantification) with amplicons in solutions at concentration of lower than about 10 nM, of lower than about 1 nM, preferably of lower than about 0.1 nM and more preferably of lower than about 0.01 nM (=1 fmole/100 μl).

In another aspect of this embodiment of the invention, very concentrated capture nucleotide sequences are used on the surface. The density of capture nucleotide sequences bound to the surface at a specific location is preferably higher than about 10 fmoles, and preferably higher than about 100 fmoles per cm² of solid support surface. If the amount of capture nucleotides is too low, the yield of the binding is quickly lower and is undetectable. Concentrations of capture nucleotide sequences between about 600 and about 3,000 nM in the spotting or binding solutions are preferred. However, concentrations as low as about 100 nM still give positive results in favorable cases (when the yield of covalent fixation is high or when the target to be detected is single stranded and present in high concentrations). Such low spotting concentrations would give density of capture nucleotide sequence as low as 20 fmoles per cm². On the other hand, higher density was only limited in the assays by the concentrations of the capture solutions, but concentrations still higher than 3,000 nM give good results.

The use of these very high concentrations and long probes are two unexpected characteristic features of the invention. The theory of DNA hybridization proposed that the rate of hybridization between two DNA complementary sequences in solution is proportional to the square root of the DNA length, the smaller one being the limited factor (Wetmur, J. G. and Davidson, N. 1968 J. Mol. Biol. 3:584). In order to obtain the required specificity, the specific sequences of the capture nucleotide sequences had to be small or limited in length compared to the target. Moreover, the targets were obtained after PCR amplification and were double stranded so that they reassociate in solution much faster than they hybridise on small sequences fixed on a solid support where diffusion is low thus reducing even more the rate of reaction. It was unexpected to observe a so large increase in the yield of hybridisation with the same short specific sequence.

In one embodiment, the amount of a target which “binds” on the spots is very small compared to the amount of capture nucleotide sequences present. So there is a large excess of capture nucleotide sequence and there was no reason to obtain the binding if even more capture nucleotide sequences.

One may perform the detection on the full length sequence after amplification or copying and when labeling is performed by incorporation of labeled nucleotides, more signal is present on the hybridized target making the assay sensitive. Since the method is highly sensitive, the capture probes are also able to capture cut target amplified sequence very efficiently. Cutting the sequences is preferably performed by enzymatic digestion, such as the DNAase, or by chemical treatment, such as the heating in alkaline solution.

The method, kit and apparatus according to this embodiment of the invention may comprise the use of other bound capture nucleotide sequences, which may have the same characteristics as the previous ones and may be used to identifying a target from another group of homologous sequences (preferably amplified by common primer(s)).

In the microbiological field, one may use the present invention for the amplification-detection of various microorganisms from the same genus or from different genuses and then identify the species by using capture nucleotide sequences of the invention. The finding of specific sequence is best performed by alignment programs using software on DNA or genomic sequences data bases. Given the genome programs of sequencing the different pathogenic organisms, it is feasible to find specific sequences for the amplification by specific primers and for the detection on specific probes. Detection of other sequences can be advantageously performed on the same array i.e. by allowing a hybridization with a standard nucleotide sequence used for the quantification or for positive or negative controls of hybridization. Said other capture nucleotide sequences have (possibly) a specific sequence longer than 10 to 60 bases and a total length as high as 600 bases and are also bound upon the insoluble solid support (preferably in the array made with the other bound capture nucleotide sequences related to the invention). A long capture nucleotide sequence may also be present on the array as consensus capture nucleotide sequence for hybridization with all sequences of the microorganisms from the same family or genus, thus giving the information on the presence or not of a microorganism of such family, genus in the biological sample.

The same array can also bear capture nucleotide sequences specific for a bacterial group (Gram positive or Gram negative strains or even all the bacteria).

The solid support according to the invention can be or can be made with materials selected from the group consisting of gel layers, glasses, electronic devices, silicon or plastic support, polymers, compact discs, metallic supports or a mixture thereof (see EP 0 535 242, U.S. Pat. No. 5,736,257, WO99/35499, U.S. Pat. No. 5,552,270, etc). Advantageously, said solid support is a single glass slide which may comprise additional means (barcodes, markers, etc.) or media for improving the method according to the invention. In a particular embodiment, the insoluble solid support is in the form of a multiwell plate.

In another particular embodiment, the different capture molecules are immobilized on different beads and, more preferably, the different beads with different capture molecules are labeled so as to be discriminated from each other. This is best achieved by using a mixture of beads having particular features, usually a particular fluorescent emission spectra, and distinguishable from each other in order to quantify the bound molecules on a particular bead. In this embodiment, one bead or a population of beads is then considered as a spot having a capture molecule specific of one target molecule.

The amplification step used in the method according to the invention is advantageously obtained by well known amplification protocols, preferably selected from the group consisting of PCR, RT-PCR, LCR, CPT, NASBA, ICR, Avalanche DNA techniques or isothermal amplification.

One particular isothermal amplification method which is suitable for RNA is the WT-Ovation™ Pico RNA Amplification System based on the Ribo-SPIA™ technology (NuGEN, San Carlos, Calif., USA). It produces amplified cDNA from total RNA for gene expression analysis. Amplification is initiated at the 3′ end as well as randomly throughout the whole transcriptome in the sample.

Ribo-SPIA™ technology is a three-step process; (1) Generation of First strand cDNA, (2) Generation of a DNA/RNA Heteroduplex Double Strand cDNA and (3) SPIA™ Amplification.

First, the cDNA is generated from the RNA by reverse transcription using a mix of DNA/RNA chimeric poly T primer and DNA/RNA chimeric random primer. These chimeric primers contain a sequence part which is common to all primers. The second part of the sequences each has a DNA portion that hybridizes either to the 5′ portion of the poly (A) sequence or randomly across the transcript. RT extends the 3′ DNA end of each primer generating first strand cDNA. The resulting cDNA/mRNA hybrid molecule contains a unique RNA sequence at the 5′ end common for all of the cDNA strands. Fragmentation of the mRNA within the cDNA/mRNA complex creates priming sites for DNA polymerase to synthesize a second strand, which includes DNA complementary to the 5′ unique sequence from the first strand chimeric primers. The result is a double stranded cDNA with a unique DNA/RNA heteroduplex at one end. RNase H is then used to degrade RNA in the DNA/RNA heteroduplex at the 5′ end of the first cDNA strand. This results in the exposure of a DNA sequence that is available for binding a second SPIA™ DNA/RNA chimeric primer. DNA polymerase then initiates replication at the 3′ end of the primer, displacing the existing forward strand. The RNA portion at the 5′ end of the newly synthesized strand is again removed by RNase H, exposing part of the unique priming site for initiation of the next round of cDNA synthesis.

The process of SPIA™ DNA/RNA primer binding, DNA replication, strand displacement and RNA cleavage is repeated, resulting in rapid accumulation of cDNA with sequence complementary to the original mRNA. The method is a linear amplification and the company claim an amplification of 15,000-fold. The method is applicable on 500 pg of starting total RNA. The method is especially well suited for the amplification of small RNA as present in paraffin embedded tissues.

Advantageously, the target to be identified is labeled previously to its hybridization with the single stranded capture nucleotide sequences. Said labeling (with known techniques from the person skilled in the art) is preferably also obtained during the amplification step. Hybridization on capture probes preferably requires the denaturation of the double stranded amplified target sequences. However, the inventors have found that this denaturation is not mandatory and hybridization can take place even without the denaturation step.

Advantageously, the length of the target is selected as being of a limited length preferably between about 60 and about 200 bases, preferably between about 80 and about 400 bases and more preferably between about 80 and about 800 bases. This preferred requirement depends on the possibility to find specific primers to amplify the required sequences possibly present in the sample. Too long target may reallocate faster and adopt secondary structures which can inhibit the fixation on the capture nucleotide sequences.

In a particular embodiment, the detection and/or the quantification of the amplified target sequences is obtained after their hybridization on corresponding capture probes in the amplification solution. Preferably, the amplification and the detection are performed in the same closed device. In a particular embodiment, the detection of the amplified sequences is performed during the PCR cycles. The amplification is preferably a real time PCR.

In a preferred embodiment, the detection of the presence of pathogenic organisms (being or not micro organisms such as bacteria or viruses) is obtained by detection of their genomic DNA sequences.

In a preferred embodiment, the detection of the presence of Genetically Modified Organisms (GMO) is performed by the detection of their genomic DNA sequences.

In another embodiment, the method is used for detection of the presence of mutations or deletions in some specific parts of a genome or in genes.

In a preferred embodiment, the original sequence to be detected and/or quantified in the sample belongs to the cytochrome P450 forms family.

Detection of genes is also a preferred application of this invention. In one embodiment the detection of homologous genes is obtained by first reverse transcription of the mRNA and then amplification by specific and universal primers as described in this invention. More particularly, the original nucleotide sequences to be detected and/or be quantified are RNA sequences submitted to a reverse transcription of the 3′ or 5′ end by using poly dT oligonucleotide or random primers of 6 or 8 or even 10 bases long. In another embodiment, the amplification is obtained by using random primers of between 6, 8 or 10 nucleotides long useful when the mRNAs present in the sample are the result of a degradation of the RNA transcripts and are found in small fragments.

In yet another embodiment, the amplification is the result of an isothermal amplification. In another embodiment, the amplification is a linear amplification. In a specific embodiment, one common sequence is used for all of the different specific primers used for the amplification of the RNA present in the sample as proposed by WT-Ovation™ Pico RNA Amplification System of NuGEN (San Carlos, Calif.).

More specifically the invention is related to a method for identifying and/or quantifying at least 5 transcripts of a cell in a sample comprising the steps of:

producing derived sequences from the parts of the transcript sequences present in the cell extract by incorporation of at least one common sequence in said parts of transcript sequences in order to obtain a partial homology between the said derived nucleotide sequences,

amplifying said derived nucleotide sequences as to produce full-length target nucleotide sequences having between 50 and 800 bases;

contacting said full-length target nucleotide sequences resulting from the amplifying step with at least 5 different single-stranded capture nucleotide sequences having between 55 and 800 bases, preferably between about 200 and about 450 bases, said single-stranded capture nucleotide sequences being covalently bound in an microarray to insoluble solid support(s) and said capture nucleotide sequences comprising a nucleotide sequence of at least 15 bases which is able to specifically bind to said full-length target nucleotide sequence, and said specific sequence is separated from the surface of the solid support by a nucleotide sequence of at least 40 bases in length; and

detecting specific hybridization of said target nucleotide sequence to said capture nucleotide sequences and quantifying the transcript expression level in the cell.

The present method allows best the detection and quantification of at least 10, preferably at least 20, and even more preferably more than 50 gene transcripts. In a preferred embodiment, the detection and/or quantification of the nucleotide sequence is performed on degraded RNA extracted from paraffin embedded tissue.

Because of the degradation of the RNA, the full length target amplified sequences are best produced by random primers so that the sequence which is amplified may be any part of the transcripts. Since their concentration is low, a first amplification step based on the use of random primer is necessary. The length of the capture molecule which gives the best reproducible and sensitive assay from one sample to the other is a sequence between about 55 and about 800 nucleotide long, preferably between about 200 and about 450 nucleotide long. The inventors have found that the use of long probes complementary to the transcripts gives very efficient, sensitive and reproducible from one sample to the other method for the detection of the cDNA coming from the small RNA present in the paraffin embedded tissues. The levels of the detection signals are also very high and well adapted for the determination of the transcripts pattern in the tissues even with analysis performed on small fragments of such transcripts. The particular feature of the method is the possibility to obtain a quantification of a particular transcript from the detection of the amplified sequences from RNA present in the tissue as small fragments which are randomly produced so that there is a collection of different fragments for each transcript.

In a preferred embodiment, the different single-stranded capture nucleotide sequences bound to the support have their entire sequences complementary or identical to one part of the transcript sequence to be detected.

In a particular embodiment, the capture nucleotide sequences comprise a nucleotide sequence of at least about 50 bases which is able to specifically bind to said full-length target nucleotide sequence without binding to said at least 4 other derived nucleotide sequences.

In a particular embodiment, the detection and/or quantification of the nucleotide sequence is performed on target amplified cDNA having a full length of between about 50 and about 150 bases long.

In a particular embodiment, the full-length target nucleotide sequences are double stranded DNA produced by PCR. In a particular embodiment, the full-length target nucleotide sequences are single stranded DNA produced by isothermal amplification.

According to a further aspect of the present invention, the method, kit (device) or apparatus according to the invention is advantageously used for the identification of different bacterial species belonging to different genus among them, Salmonella, Escherichia coli, Yersinia, Vibrio, Enterobacterium, Pseudomonas.

According to a further aspect of the present invention, the method, kit (device) or apparatus according to the invention is advantageously used for the identification of at least 5 GMO obtained after amplification of one of their DNA sequences with specific primers and detection on specific capture molecules present on an array.

The present method also allows the detection of antibiotic resistance genes or genetic variants such as the FemA gene, the Gyrase gene or the MexR gene.

The method of the present invention allows the detection of mutations or deletions in some specific parts of a genome or in genes for the polymorphism analysis of a genome or particular genes. Examples of polymorphism are given in example 5 on the genes gyrase and muxR related to antibiotic resistance. Detection of polymorphism is especially useful for the detection of genetic diseases and for analyzing specific susceptibilities of patients to drugs, such as a cytochrome P450, where the presence of certain isoforms modifies the metabolism of some drugs.

Another aspect of the present invention is related to any part of biochips or microarray comprising said above described sequences (especially the specific capture nucleotide sequence described in the examples) as well as a general screening method for the identification of a target sequence specific of said microorganisms of family type discriminated from other sequences upon any type of microarrays or biochips by any method.

After hybridization on the array, the target sequence is detected by any current techniques suitable for micro detection on arrays or on equivalent support. Without labelling, preferred methods are the identification of the target by mass spectrometry now adapted to the arrays (U.S. Pat. No. 5,821,060) or by intercalating agents followed by fluorescent detection (WO97/27329 or Fodor et al. 1993 Nature 364: 555).

The detection methods employing labels are numerous. A review of the different labeling molecules is given in WO 97/27317. They are obtained using either already labeled primer or by incorporation of labeled nucleotides during the copying or amplification step. Labeling can also be obtained by ligating a detectable moiety onto the RNA or DNA to be tested (a labeled oligonucleotide, which is ligated, at the end of the sequence by a ligase). Fragments of RNA or DNA can also incorporate labeled nucleotides at their 5′OH or 3′OH ends using a kinase, a transferase or a similar enzyme.

The most frequently used labels are fluorochromes like Cy3, Cy5 and Cy7 suitable for analyzing an array by using commercially available array scanners (General Scanning, Genetic Microsystem, etc.). Radioactive labeling, cold labeling or indirect labeling with small molecules recognized thereafter by specific ligands (streptavidin or antibodies) are common methods. The resulting signal of target fixation on the array is either fluorescent, calorimetric, diffusion, electroluminescent, bio- or chemiluminescent, magnetic, electric like impedometric or voltammetric (U.S. Pat. No. 5,312,527). A preferred method is based upon the use of the gold labeling of the bound target in order to obtain a precipitate or silver staining which is then easily detected and quantified by a scanner.

Quantification has to take into account not only the hybridization yield and detection scale on the array (which is identical for target and reference sequences) but also the extraction, the amplification (or copying) and the labeling steps.

The method according to the invention may also comprise means for obtaining a quantification of target nucleotide sequences by using a standard nucleotide sequence (external or internal standard) added at known concentration. A capture nucleotide sequence is also present on the array so as to hybridize to the standard in the same conditions as said target (possibly after amplification or copying). In this embodiment, the method comprises the quantification of a signal resulting from the formation of a double stranded nucleotide sequence formed by complementary base pairing between the capture nucleotide sequences and the standard and the step of a correlation analysis of signal resulting from the formation of said double stranded nucleotide sequence with the signal resulting from the double stranded nucleotide sequence formed by complementary base pairing between capture nucleotide sequence(s) and the target in order to quantify the presence of the original nucleotide sequence to be detected and/or quantified in the biological sample.

Advantageously the standard is added in the initial biological sample or after the extraction step and is amplified or copied with the same primers and/or has a length and a GC content identical or differing by no more than 20% from the target. More preferably, the standard can be designed as a competitive internal standard having the characteristics of the internal standard found in the document WO98/11253, the disclosure of which is incorporated herein by reference in its entirety. Said internal standard has a part of its sequence common to the target and a specific part which is different. It also has at or near its two ends sequences which are complementary of the two primers used for amplification or copy of the target and similar GC content (WO98/11253).

Preferably, the hybridization yield of the standard through this specific sequence is identical or differs by no more than 20% from the hybridization yield of the target sequence and quantification is obtained as described in WO 98/11253.

Said standard nucleotide sequence, external and/or internal standard, is also advantageously included in the kit (device) or apparatus according to the invention, possibly with all the media and means necessary for performing the different steps according to the invention (hybridization and incubation media, polymerase and other enzymes, standard sequence(s), labeling molecule(s), etc.).

The present invention also covers the means for performing the method. Particularly, the invention includes a diagnostic and/or quantification kit which comprises:

an insoluble solid support upon which single stranded capture nucleotide sequences are bound in an array, said single stranded capture nucleotide sequences containing a sequence of between about 10 and about 600 bases, and preferably between 50 and 450 bases specific for a target nucleotide sequence to be detected and/or quantified and having a total length comprised between about 30 and about 800 600 bases comprising a spacer having a nucleotide sequence of at least about 20 bases and preferably of at least about 40 bases and, in some embodiments even longer than about 60 bases, said single stranded capture nucleotide sequences being disposed upon the surface of the solid support according to an array with a density of at least 4 single stranded capture nucleotide sequences/cm² of the solid support, and.

an amplification (PCR) solution that comprises at least 5 different target specific primers and a universal primer pair, a thermostable DNA polymerase, a plurality of dNTPs and a buffered solution having a pH comprised between 7 and 9 for containing the primers.

In a preferred embodiment, the kit comprises a device having a chamber for performing the amplification reaction together with detection and possibly a quantification of amplified target sequences. The kit preferably comprises the amplification reagents for the performance of the PCR amplification together with the hybridization on the immobilized capture molecules.

In another embodiment, the insoluble solid support of the kit is selected from the group consisting of glasses, electronic devices, silicon supports, plastic supports, compact discs, gel layers, metallic supports or a mixture thereof.

In another preferred embodiment of the kit, the single stranded capture nucleotide sequences are disposed upon the surface of the solid support as an array with a density of at least 4 single stranded capture nucleotide sequences/cm² of the solid support surface.

In another embodiment, the insoluble solid support of the kit is in the form of a multiwell plate.

In another preferred embodiment, the insoluble solid support is a series of microbeads. The biochip is composed of a collection of beads on which the capture molecules are bound with one particular bead having only one capture molecule sequence. The beads are labeled so that they can be recognized preferably by a bead analyzed and counter such as the FACS machine.

In a preferred embodiment of the kit, the capture nucleotide sequences are specific to a target nucleotide sequence to be detected and/or quantified which is specific for a gene selected from the group consisting of bacteria, human cells, cytochrome P450 forms family.

In another embodiment, the diagnostic kit comprises biochips, for identification and/or quantification of GMOs obtained after amplification of one of their DNA sequences with specific primers and detection on specific capture molecules present on an array. In some embodiments, the kit allows identification and/or quantification of at least 5 GMOs. Preferably the specific capture molecules present on an array contain at least 5 bases located on either sides of the 3′ or 5′ flanking regions of the foreign DNA incorporated into the genome of the plant in order to obtain a of the GMO.

In a specific embodiment, the diagnostic kit comprises biochips, for identification and/or quantification of bacterial species obtained after amplification of one of their DNA sequences with specific primers and universal primer(s) and detection on an array. In some embodiments, the kit allows the identification and/or quantification of at least 5 bacterial species.

In another preferred embodiment, the diagnostic kit comprises biochips, for identification and/or quantification of different single nucleotide polymorphism (SNP) located at different locations in the genome of an organism.

In another embodiment, the kit comprises biochips, for identification and/or quantification of at least 5 gene transcripts obtained after amplification of one of their RNA or cDNA sequences with specific primer(s) and detection on specific capture molecules present on an array.

Advantageously, the biochips also contain spots with various concentrations (e.g., 4) of labeled capture nucleotide sequences. These labeled capture nucleotide sequences are spotted from known concentrations solutions and their signals allow the conversion of the results of hybridization into absolute amounts. They also allow testing for the reproducibility of the detection.

In a particular embodiment, the support for the capture molecules is a multiwell plate.

Alternatively, the biochip is composed of a collection of beads on which the capture molecules are bound with one particular bead having only one capture molecule sequence. The beads are labeled so that they can be recognized preferably by a bead analyzed and counter such as the FACS machine.

The solid support (biochip) can be inserted in a support connected to another chamber and automatic machine through the control of liquid solution based upon the use of microfluidic technology. By being inserted into such a microlaboratory system, it can be incubated, heated, washed and labeled by automates, even for previous steps (like extraction of DNA, amplification by PCR) or the following step (labeling and detection). All these steps can be performed upon the same solid support. In a preferred embodiment, the mixing is performed by movement of the liquid by physical means such as pump, opening and closing valves, electrostatic waves or piezoelectric vibrations

Preferably the support containing the capture molecules is part of a device having a chamber for performing the amplification reaction and a chamber having capture molecules for performing the hybridization and the detection of the target molecules.

Preferably the chamber for performing the PCR reaction is in a material resistant to 95° C. Preferably material is selected from the group consisting of glass, polymer, polycarbonate (PC), polyethylene (PE), Cycloolefin copolymer (COC), cyclic olefin polymer (COP and a mixture thereof. In still another embodiment, the chamber for PCR has a thickness of material of less than 2 mm and better less than 1 mm.

In a preferred embodiment, the incubation system provides conditions so that the thickness of the solution being in contact with the micro-array is constant above all the arrayed spots or localized areas. The difference of thickness between two spots or localized areas of the arrayed surface is preferably lower than 100 micrometers and may be lower than 10 micrometers and/or even lower than 1 micrometer. In another embodiment, the incubation system provides conditions for the thickness of the solution which is in contact with the micro-array to be changed between two measurements. In still another embodiment the chamber having the capture molecules has a surface having a transmission of more than 90% and better more than 95% at a the wavelength of detection of the target label. In still another embodiment the chamber having the capture molecules has a surface having allowing the same detection efficiency on the overall surface covered by the micro-array to be analyzed.

Preferably the detection and/or the quantification of the amplified target sequences is obtained after their hybridization on corresponding capture probes in the amplification solution.

In still another embodiment the PCR chamber and the array chambers are the same chamber.

In a particular embodiment, the amplification and the detection are performed in the same closed device. In still another embodiment, the detection of the amplified sequences is performed during the PCR cycles and preferably the detection is a real time PCR.

The present invention will be described in details in the following non-limiting examples in reference to the enclosed figures.

### EXAMPLE 1

### Detection of Homologous FemA Sequences on Array Bearing Long Specific Capture Nucleotide Sequences

Production of the Capture Nucleotide Sequences and of the Targets

The FemA genes corresponding to the different Staphylococci species were amplified separately by PCR using the following primers:

S. aureus 1:

(SEQ ID NO: 1)

5′ CTTTTGCTGATCGTGATGACAAA 3′;
S. aureus 2:

(SEQ ID NO: 2)

5′ TTTATTTAAAATATCACGCTCTTCG 3′;
S. epidermidis 1:

(SEQ ID NO: 3)

5′ TCGCGGTCCAGTAATAGATTATA 3′;
S. epidermidis 2:

(SEQ ID NO: 4)

5′ TGCATTTCCAGTTATTTCTCCC 3′;
S. haemolyticus 1:

(SEQ ID NO: 5)

5′ ATTGATCATGGTATTGATAGATAC 3′;
S. haemolyticus 2:

(SEQ ID NO: 6)

5′ TTTAATCTTTTTGAGTGTCTTATAC 3′;
S. saprophyticus 1:

(SEQ ID NO: 7)

5′ TAAAATGAAACAACTCGGTTATAAG 3′;
S. saprophyticus 2:

(SEQ ID NO: 8)

5′ AAACTATCCATACCATTAAGTACG 3′;
S. hominis 1:

(SEQ ID NO: 9)

5′ CGACCAGATAACAAAAAAGCACAA 3′;
S. hominis 2:

(SEQ ID NO: 10)

5′ GTAATTCGTTACCATGTTCTAA 3′.

The PCR was performed in a final volume of 50 μl containing: 1.5 mM MgCl₂, 10 mM Tris pH 8.4, 50 mM KCl, 0.8 μM of each primer, 50 μM of each dNTP, 50 μM of biotin-16-dUTP), 1.5 U of Taq DNA polymerase Biotools, 7.5% DMSO, 5 ng of plasmid containing FemA gene. Samples were first denatured at 94° C. for 3 min. Then 40 cycles of amplification were performed consisting of 30 sec at 94° C., 30 sec at 60° C. and 30 sec at 72° C. and a final extension step of 10 min at 72° C. Water controls were used as negative controls of the amplification. The sizes of the amplicons obtained using these primers were 108 bp for S. saprophyticus, 139 bp for S. aureus, 118 bp for S. hominis, 101 bp for S. epidermidis and 128 bp for S. haemolyticus. The sequences of the capture nucleotide sequences were the same as the corresponding amplicons but they were single strands.

The biochips also contained positive controls which were CMV amplicons hybridized on their corresponding capture nucleotide sequence and negative controls which were capture nucleotide sequences for a HIV-I sequence on which the CMV could not bind.

Capture Nucleotide Sequence Immobilization

The protocol described by Schena et al. (1996 PNAS. USA 93:10614) was followed for the grafting of aminated DNA to aldehyde derivatized glass. The aminated capture nucleotide sequences were spotted from solutions at concentrations ranging from 150 to 3000 nM. The capture nucleotide sequences were printed onto the silylated microscopic slides with a home made robotic device (250 μm pins from Genetix (UK) and silylated (aldehyde) microscope slides from Cell associates (Houston, USA)). The spots have 400 μm in diameter and the volume dispensed is about 0.5 nl. Slides were dried at room temperature and stored at 4° C. until used.

Hybridization

At 65 μl of hybridization solution (AAT, Namur, Belgium) were added 5 μl of amplicons and the solution was loaded on the array framed by a hybridization chamber. For positive controls we added 2 nM biotinylated CMV amplicons of 437 bp to the solution; their corresponding capture nucleotide sequences were spotted on the array. The chamber was closed with a coverslip and slides were denatured at 95° C. for 5 min. The hybridization was carried out at 600 for 2 h. Samples were washed 4 times with a washing buffer.

Colorimetric Detection

The glass samples were incubated 45 min at room temperature with 800 μl of streptavidin labeled with colloidal gold 1000× diluted in blocking buffer (Maleic buffer 100 mM pH 7.5, NaCl 150 mM, Gloria milk powder 0.1%). After 5 washes with washing buffer, the presence of gold served for catalysis of silver reduction using a staining revelation solution (AAT, Namur, Belgium). The slides were incubated 3 times 10 min with 800 μl of revelation mixture, then rinsed with water, dried and analyzed using a microarray reader. Each slide was then quantified by a specific quantification software.

Fluorescence Detection

The glass samples were incubated 45 min at room temperature with 800 μl of Cyanin 3 or Cyanin 5 labeled streptavidin. After washing, the slides were dried before being stored at room temperature. The detection was performed in the array-scanner GSM 418 (Genetic Microsystem, Woburn, Mass., USA). Each slide was then quantified by a specific quantification software.

The results give a cross-reaction between the species. For example, epidermidis amplicons hybridized on its capture nucleotide sequence give a value of 152, but give a value of 144, 9, 13 and 20 respectively for the S. saprophyticus, S. aureus, S. haemolyticus and S. hominis capture nucleotide sequences.

### EXAMPLE 2

### Detection of Homologous FemA Sequences on Array Bearing Small Specific Capture Nucleotide Sequences

Protocols for capture nucleotide sequences immobilization and silver staining detection were described in Example 1 but the capture nucleotide sequences specific of the 5 Staphylococcus species were spotted at concentrations of 600 nM and are the following:

| Name | |
|---|---|
| Capture | |
| nucleotide | |
| sequence | Sequence (5′ -> 3′) |
| ATaur02 | ATTTAAAATATCACGCTCTTCGTTTAG |
| | (SEQ ID NO: 11) |
| ATepi02 | ATTAAGCACATTTCTTTCATTATTTAG |
| | (SEQ ID NO: 12) |
| AThae02 | ATTTAAAGTTTCACGTTCATTTTGTAA |
| | (SEQ ID NO: 13) |
| AThom02 | ATTTAATGTCTGACGTTCTGCATGAAG |
| | (SEQ ID NO: 14) |
| ATsap02 | ACTTAATACTTCGCGTTCAGCCTTTAA |
| | (SEQ ID NO: 15) |

In this case, the targets are fragments of the FemA gene sequence corresponding to the different Staphylococci species which were amplified by a PCR using the following consensus primers:

APstap03:

(SEQ ID NO: 16)

5′ CCCACTCGCTTATATAGAATTTGA 3′;
APstap04:

(SEQ ID NO: 17)

5′ CCACTAGCGTACATCAATTTTGA 3′;
APstap05:

(SEQ ID NO: 18)

5′ GGTTTAATAAAGTCACCAACATATT 3′.

This PCR was performed in a final volume of 100 μl containing: 3 mM MgCl₂, 1 mM Tris pH 8, 1 μM of each primer, 200 μM of dATP, dCTP and dGTP, 150 μM of dTTP, 50 μM of biotin-16-dUTP, 2.5 U of Taq DNA polymerase (Boehringer Mannheim, Allemagne), 1 U of Uracil-DNA-glycosylase heat labile (Boehringer Mannheim, Allemagne), 1 ng of plasmid containing FemA gene. Samples were first denatured at 94° C. for 5 min. Then 40 cycles of amplification were performed consisting of 1 min at 94° C., 1 min at 50° C. and 1 min at 72° C. and a final extension step of 10 min at 72° C. Water controls were used as negative controls of the amplification. The sizes of the amplicons obtained using these primers were 489 bp for all species.

The hybridization solution was prepared as in example 1 and loaded on the slides. Slides were denatured at 98° C. for 5 min. Hybridization is carried out at 50° C. for 2 h. Samples are then washed 4 times with a washing buffer. The values were very low and almost undetectable.

### EXAMPLE 3

### Effect of the Spacer Length on the Sensitivity of Detection of Homologous FemA Sequences on Array Bearing Long Capture Nucleotide Sequences with a Small Specific Sequence

The experiment was conducted as described in Example 2 with the same amplicons but the capture nucleotide sequences used are the following:

| Name | |
|---|---|
| Capture | |
| nucleotide | |
| sequence | Sequence (5′ -> 3′) |
| Ataur02 | ATTTAAAATATCACGCTCTTCGTTTAG |
| | (SEQ ID NO: 11) |
| ATepi02 | ATTAAGCACATTTCTTTCATTATTTAG |
| | (SEQ ID NO: 12) |
| ATepi03 | GAATTCAAAGTTGCTGAGAAATTAAGCACATTTCTTTCA |
| | TTATTTAG |
| | (SEQ ID NO: 19) |
| ATepi04 | GAATTCAAAGTTGCTGAGAATAGTTCAATGGAAGGAAGC |
| | GATTAAGCACATTTCTTTCATTATTTAG |
| | (SEQ ID NO: 20) |
| ATepi05 | GAATTCAAAGTTGCTGAGAATAGTTCAATGGAAGGAAGC |
| | GTCTTCTTAAAATCTAAAGAAATTAAGCACATTTCTTTC |
| | ATTATTTAG |
| | (SEQ ID NO: 21) |

^{a}The spacer sequences are underlined

The target amplicons were 489 bp long while the capture nucleotide sequences were 47, 67 or 87 bases single stranded DNA with a specific sequence of 27 bases.

### EXAMPLE 4

### Specificity of the Detection of FemA Sequences from Different Bacterial Species on the Same Array Bearing Long Capture Nucleotide Sequences with a Small Specific Sequence

The experiment was conducted as described in example 2 but the capture nucleotide sequences were spotted at concentrations of 3000 nM and are the following:

| Name | |
|---|---|
| Capture | |
| nucleotide | |
| sequence | Sequence (5′ -> 3′) |
| Ataur27 | GAATTCAAAGTTGCTGAGAATAGTTCAATGGAAGGAAGC |
| | GATTTAAAATATCACGCTCTTCGTTTAG |
| | (SEQ ID NO: 22) |
| Atepi27 | GAATTCAAAGTTGCTGAGAATAGTTCAATGGAAGGAAGC |
| | GATTAAGCACATTTCTTTCATTATTTAG |
| | (SEQ ID NO: 23) |
| Athae27 | GAATTCAAAGTTGCTGAGAATAGTTCAATGGAAGGAAGC |
| | GATTTAAAGTTTCACGTTCATTTTGTAA |
| | (SEQ ID NO: 24) |
| Athom27 | GAATTCAAAGTTGCTGAGAATAGTTCAATGGAAGGAAGC |
| | GATTTAATGTCTGACGTTCTGCATGAAG |
| | (SEQ ID NO: 25) |
| Atsap27 | GAATTCAAAGTTGCTGAGAATAGTTCAATGGAAGGAAGC |
| | GACTTAATACTTCGCGTTCAGCCTTTAA |
| | (SEQ ID NO: 26) |

^{a}The spacer sequence is underlined. The specific sequences were of 27 bases

The targets are fragments of the FemA gene sequence corresponding to the different Staphylococci species which were amplified by PCR using the following consensus primers:

APcons3-1:

(SEQ ID NO: 27)

5′ TAAYAAARTCACCAACATAYTC 3′;
APcons3-2:

(SEQ ID NO: 28)

5′ TYMGNTCATTTATGGAAGATAC 3′

A consensus sequence is present on the biochips which detects all the tested Staphylococcus species. All target sequences were amplified by PCR with the same pair of primers.

The size of the amplicons obtained using these primers were 587 bp for all species. The consensus sequence capture nucleotide sequence was a 489 base long single stranded DNA complementary to the amplicons of S. hominis as amplified in example 2. The detection was made in fluorescence. Homology between the consensus capture nucleotide sequence and the sequences of the FemA from the 15 S. species were between 66 and 85%. All the sequences hybridized on this consensus capture nucleotide sequence.

### EXAMPLE 5

### Effect of the Length of the Specific Sequence of the Capture Nucleotide Sequence on the Discrimination Between Homologous Sequences

The experiment was conducted as described in example 4 but at a temperature of 43° C. and the capture nucleotide sequences used are presented in the table here joined. The numbers after the names indicate the length of the specific sequences.

The FemA amplicons of S. anaerobius (a subspecies of S. aureus) were hybridized on an array bearing capture nucleotide sequences of 67 single stranded bases with either 15, 27 and 40 bases specific for the S. aureus, anaerobius and epidermidis at their extremities. The difference between the capture nucleotide sequences of anaerobius and aureus was only one base in the 15 base capture nucleotide sequence and 2 in the 27 and the 40 bases.

The amplicons of the FemA from the three Staphylococcus species were hybridized on the array.

| Name | |
|---|---|
| Capture | |
| nucleotide | |
| sequence | Sequence (5′ -> 3′) |
| Ataur15 | GAATTCAAAGTTGCTGAGAATAGTTCAATGGAAGGAAGC |
| | GTCTTCTTAAAATGCTCTTCGTTTAGTT |
| | (SEQ ID NO: 29) |
| Ataur27 | GAATTCAAAGTTGCTGAGAATAGTTCAATGGAAGGAAGC |
| | GATTTAAAATATCGCTCTTCGTTTAG |
| | (SEQ ID NO: 22) |
| Ataur40 | GAATTCAAAGTTGCTGAGAATAGTTCAAATCTTTATTTA |
| | AAATATCACGCTCTTCGTTTAGTTCTTT |
| | (SEQ ID NO: 30) |
| Atana15 | GAATTCAAAGTTGCTGAGAATAGTTCAATGGAAGGAAGC |
| | GTCTTCTTAAAATGCTCTTCATTTAGTT |
| | (SEQ ID NO: 31) |
| Atana27 | GAATTCAAAGTTGCTGAGAATAGTTCAATGGAAGGAAGC |
| | GGTTTAAAATATCACGCTCTTCATTTAG |
| | (SEQ ID NO: 32) |
| Atana40 | GAATTCAAAGTTGCTGAGAATAGTTCAAATCTTTGTTTA |
| | AAATATCACGCTCTTCATTTAGTTCTTT |
| | (SEQ ID NO: 33) |
| Atepi15 | GAATTCAAAGTTGCTGAGAATAGTTCAATGGAAGGAAGC |
| | GTCTTCTTAAAATTTTCATTATTTAGTT |
| | (SEQ ID NO: 34) |
| Atepi27 | GAATTCAAAGTTGCTGAGAATAGTTCAATGGAAGGAAGC |
| | GATTAAGCACATTTCTTTCATTATTTAG |
| | (SEQ ID NO: 23) |
| Atepi40 | GAATTCAAAGTTGCTGAGAATAGTTCAAATCTTTATTAA |
| | GCACATTTCTTTCATTATTTAGTTCCTC |
| | (SEQ ID NO: 35) |

### EXAMPLE 6

### Sensitivity of the Detection of FemA Sequences of Staphylococcus aureus on Arrays Bearing Specific Sequence as Proposed by this Invention and the Consensus Sequence

The experiment was conducted as described in example 4 with the capture nucleotide sequences spotted at concentrations of 3000 nM. The bacterial FemA sequences were serially diluted before the PCR and being incubated with the arrays.

### EXAMPLE 7

### Detection of 16 Homologous FemA Sequences on Array

The consensus primers and the amplicons were the same as described in the example 4 but the capture probes were chosen for the identification of 15 Staphylococcus species. The experiment is conducted as in example 4. The capture nucleotide sequences contain a spacer fixed on the support by its 5′ end and of the following sequence 5′ GAATTCAAAGTTGCTGAGAATAGTTCAATGGAAGGAAGCG 3′ (SEQ ID NO: 36) followed by the following specific sequences for the various femA from the different Staphylococcus:

S. aureus:

(SEQ ID NO: 37)

ATTTAAAATATCACGCTCTTCGTTTAG;
S. epidermidis:

(SEQ ID NO: 38)

ATTAAGCACATTTCTTTCATTATTTAG;
S. haemolyticus:

(SEQ ID NO: 39)

ATTTAAAGTTTCACGTTCATTTTGTAA;
S. hominis:

(SEQ ID NO: 40)

ATTTAATGTCTGACGTTCTGCATGAAG;
S. saprophyticus:

(SEQ ID NO: 41)

ACTTAATACTTCGCGTTCAGCCTTTAA;
S. capitis:

(SEQ ID NO: 42)

ATTAAGAACATCTCTTTCATTATTAAG;
S. caseolyticus:

(SEQ ID NO: 43)

ATAAAGACATTCGAGACGAAGGCT;
S. cohnii:

(SEQ ID NO: 44)

ACTTAACACTTCACGCTCTGACTTGAG;
S. gallinarum:

(SEQ ID NO: 45)

ACTTAAAACTTCACGTTCAGCAGTAAG;
S. intermedius:

(SEQ ID NO: 46)

GTGGAAATCTTGCTCTTCAGATTTCAG;
S. lugdunensis:

(SEQ ID NO: 47)

TTCTAAAGTTTGTCGTTCATTCGTTAG
S. schleiferi:

(SEQ ID NO: 48)

TTTAAAGTCTTGCGCTTCAGTGTTGAG;
S. sciuri:

(SEQ ID NO: 49)

GTTGTATTGTTCATGTTCTTTTTCTAA;
S. simulans:

(SEQ ID NO: 50)

TTCTAAATTCTTTTGTTCAGCGTTCAA;
S. warneri:

(SEQ ID NO: 51)

AGTTAAGGTTTCTTTTTCATTATTGAG;
S. xylosus:

(SEQ ID NO: 52)

GCTTAACACCTCACGTTGAGCTTGCAA.

### EXAMPLE 8

### Detection of 13 Homologous p34 Sequences and Identification of 13 Mycobacteria Species

The P34 genes present in all Mycobacteria were all amplified with the following consensus primers:

Sense

MycU4 5′ CATGCAGTGAATTAGAACGT 3′ (SEQ ID NO: 53) located at the position 496-515 of the gene, Tm=56° C.

Antisense

APmcon02 5′ GTASGTCATRRSTYCTCC 3′ (SEQ ID NO: 54) located at the position 733-750 of the gene, Tm 52-58° C., S═C or G; R=A or G; Y=T or C.

The size of amplified products ranges from 123 to 258 bp.

The following capture nucleotide sequences were chosen for the specific capture of the Mycobacteria sequences:

Capture nucleotide sequences
M. avium:

(SEQ ID NO: 55)

5′ CGGTCGTCTCCGAAGCCCGCG 3′ (21 nt)
M. gastrii 1:

(SEQ ID NO: 56)

5′ GATCGGCAGCGGTGCCGGGG 3′ (20 nt);
M. gastrii 3:

(SEQ ID NO: 57)

5′ GTATCGCGGGCGGCAAGGT 3′ (19 nt);
M. gastrii 5:

(SEQ ID NO: 58)

5′ TCTGCCGATCGGCAGCGGTGCCGG 3′ (24 nt);
M. gastrii 7:

(SEQ ID NO: 59)

5′ GCCGGGGCCGGTATTCGCGGGCGG 3′ (24 nt)
M. gordonae:

(SEQ ID NO: 60)

5′ GACGGGCACTAGTTGTCAGAGG 3′ (22 nt);
M. intracellulare 1:

(SEQ ID NO: 61)

5′ GGGCCGCCGGGGGCCTCGCCG 3′ (21 nt);
M. intracellulare 3:

(SEQ ID NO: 62)

5′ GCCTCGCCGCCCAAGACAGTG 3′ (21 nt);
M. leprae:

(SEQ ID NO: 63)

5′ GATTTCGGCGTCCATCGGTGGT 3′ (22 nt);
M. kansasi 1:

(SEQ ID NO: 64)

5′ GATCGTCGGCAGTGGTGACGG 3′ (21 nt);
M. kansasi 3:

(SEQ ID NO: 65)

5′ TCGTCGGCAGTGGTGAC 3′ (17 nt);
M. kansasi 5:

(SEQ ID NO: 66)

5′ ATCCGCCGATCGTCGGCAGTGGTGACG 3′ (27 nt);
M. malmoense:

(SEQ ID NO: 67)

5′ GACCCACAACACTGGTCGGCG 3′ (21 nt);
M. marinum:

(SEQ ID NO: 68)

5′ CGGAGGTGATGGCGCTGGTCG 3′ (21 nt);
M. scrofulaceum:

(SEQ ID NO: 69)

5′ CGGCGGCACGGATCGGCGTC (20 nt);
M. simiae:

(SEQ ID NO: 70)

5′ ATCGCTCCTGGTCGCGCCTA 3′ (20 nt);
M. szulgai:

(SEQ ID NO: 71)

5′ CCCGGCGCGACCAGCAGAACG 3′ (21 nt);
M. tuberculosis:

(SEQ ID NO: 72)

5′ GCCGTCCAGTCGTTAATGTCGC 3′ (22 nt);
M. xenopi:

(SEQ ID NO: 73)

5′ CGGTAGAAGCTGCGATGACACG 3′ (22 nt);

Each of the sequences above comprises a spacer at its 5′ end. Spacer sequence: 5′ GAATTCAAAGTTGCTGAGAATAGTTCAATGGAAGGAAGCGTCTTC 3′ (SEQ ID NO: 74). Capture nucleotide sequences were aminated at their 5′ end.

### EXAMPLE 9

### Detection of MAGE Genes

MAGE genes were all amplified with the following consensus primers:

Sense

DPSCONS2 5′ GGGCTCCAGCAGCCAAGAAGAGGA 3′ (SEQ ID NO: 75), located at the 398-421 position of the gene, Tₘ=78° C.

Other amplicons were added as sense primer in order to increase the efficiency of the PCR for some MAGEs:

DPSMAGE1

(SEQ ID NO: 76)

5′ GGGTTCCAGCAGCCGTGAAGAGGA 3′, Tm = 78° C.;
DPSMAG8

(SEQ ID NO: 77)

5′ GGGTTCCAGCAGCAATGAAGAGGA 3′, Tm = 74° C.;
DPSMAG12

(SEQ ID NO: 78)

5′ GGGCTCCAGCAACGAAGAACAGGA 3′, Tm = 76° C.;

Antisense

DPASCONB4 5′ CGGTACTCCAGGTAGTTTTCCTGC 3′ (SEQ ID NO: 79), located at the position 913-936 of the gene, Tm=74° C.

The size of the amplified products are around 530 bp.

The following capture nucleotide sequences of 27 nucleotides were chosen for the specific capture of the MAGE sequences:

Capture nucleotide sequences
Mage 1 DTAS01

(SEQ ID NO: 80)

5′ ACAAGGACTCCAGGATACAAGAGGTGC 3′;
Mage 2 DTAS02

(SEQ ID NO: 81)

5′ ACTCGGACTCCAGGTCGGGAAACATTC 3′;
Mage 3 DTS0306

(SEQ ID NO: 82)

5′ AAGACAGTATCTTGGGGGATCCCAAGA 3′;
Mage 4 DTAS04

(SEQ ID NO: 83)

5′ TCGGAACAAGGACTCTGCGTCAGGCGA 3′;
Mage 5 DTAS05

(SEQ ID NO: 84)

5′ GCTCGGAACACAGACTCTGGGTCAGGG 3′;
Mage 6 DTS06

(SEQ ID NO: 85)

5′ CAAGACAGGCTTCCTGATAATCATCCT 3′;
Mage 7 DTAS07

(SEQ ID NO: 86)

5′ AGGACGCCAGGTGAGCGGGGTGTGTCT 3′;
Mage 8 DTAS08

(SEQ ID NO: 87)

5′ GGGACTCCAGGTGAGCTGGGTCCGGGG 3′;
Mage 9 DTAS09

(SEQ ID NO: 88)

5′ TGAACTCCAGCTGAGCTGGGTCGACCG 3′;
Mage 10 DTAS10

(SEQ ID NO: 89)

5′ TGGGTAAAGACTCACTGTCTGGCAGGA 3′;
Mage 11 DTAS11

(SEQ ID NO: 90)

5′ GAAAAGGACTCAGGGTCTATCAGGTCA 3′;
Mage 12 DTAS12

(SEQ ID NO: 91)

5′ GTGCTACTTGGAAGCTCGTCTCCAGGT 3′;

Each of the sequences above comprises a spacer aminated at its 5′ end in order to be covalently linked to the glass. Spacer sequence 5′ GAATTCAAAGTTGCTGAGAATAGTTCAATGGAAGGAAGCG 3′ (SEQ ID NO: 36).

They were spotted on aldehyde bearing glasses and used for the detection of the MAGEs amplified by the consensus primers given here above. The results showed a non equivocal identification of the MAGEs present in the tumors compared to identification using 12 specific PCR, one for each MAGE sequences.

### EXAMPLE 10

### Identification of G-Protein Dopamine Receptors Subtypes in Rat

Dopamine Receptors coupled to the G-protein were all amplified with the following consensus primers:

Sense

CONSENSUS2-3-4: 5′ TGCAGACMACCACCAACTACTT 3′ (SEQ ID NO: 92) located at the position 221-242 of the gene, Tₘ=66° C.; M=A or C;

CONSENSUS1-5: 5′ TGMGGKCCAAGATGACCAACWT 3′ (SEQ ID NO: 93) (22 nt) located at the position 221-240 of the gene, Tm=66° C.; M=A or C; K=G or T; W=A or T.

Antisense

5′ TCATGRCRCASAGGTTCAGGAT 3′ (SEQ ID NO: 94) located at the position 395-416 of the gene, Tm=64-68° C.; R=A or G; S═C or G.

The size of the amplified product is 196 bp.

The following capture nucleotide sequences of 27 nucleotides were chosen for the specific capture of the dopamine receptor sequences:

Capture nucleotide sequences
DRD1

(SEQ ID NO: 95)

5′ CTGGCTTTTGGCCTTTGGGTCCCTTTT 3′;
DRD2

(SEQ ID NO: 96)

5′ TGATTGGAAATTCAGCAGGATTCACTG 3′;
DRD3

(SEQ ID NO: 97)

5′ GAGTCTGGAATTTCAGCCGCATTTGCT 3′;
DRD4

(SEQ ID NO: 98)

5′ CGTCTGGCTGCTGAGCCCCCGCCTCTG 3′;
DRD5

(SEQ ID NO: 99)

5′ CTGGGTACTGGCCCTTTGGGACATTCT 3′.

Each of the sequences above comprised an aminated spacer at its 5′ end. Spacer sequence 5′ GAATTCAAAGTTGCTGAGAATAGTTCAATGGAAGGAAGCG (SEQ ID NO: 36).

### EXAMPLE 11

### Identification of G-Protein Histamine Receptors Subtypes in Rat

Histamine Receptors coupled to the G-protein were all amplified with the following primers:

Sense

H1sense: 5′ CTCCGTCCAGCAACCCCT 3′ (SEQ ID NO: 100) (18 nt) located at the Position 381-398 of the gene, Tm=60° C.

H2sense: 5′ CTGTGCTGGTCACCCCAGT 3′ (SEQ ID NO: 101) (19 nt) located at the Position 380-398 of the gene, Tm=62° C.

H3sense: 5′ ACTCATCAGCTATGACCGATT 3′ (SEQ ID NO: 102) (21 nt) located at the Position 378-398 of the gene, Tm=60° C.

Antisense

H1antisense: 5′ ACCTTCCTTGGTATCGTCTG 3′ (SEQ ID NO: 103) (20 nt) located at the Position 722-741 of the gene, Tm=60° C.

H2antisense: 5′ GAAACCAGCAGATGATGAACG 3′ (SEQ ID NO: 104) (21 nt) located at the Position 722-742 of the gene, Tm=62° C.

H3antisense: 5′ GCATCTGGTGGGGGTTCTG 3′ (SEQ ID NO: 105) (19 nt) located at the Position 722-740 of the gene, Tm=62° C.

Size of the amplified product ranged from 359 to 364 bp.

The following capture nucleotide sequences were chosen for the specific capture of the histamine receptor sequences:

Capture nucleotide sequences
H1

(SEQ ID NO: 106)

5′ CCCCAGGATGGTAGCGGA 3′ (18 nt);
H2

(SEQ ID NO: 107)

5′ AGGATAGGGTGATAGAAATAAC 3′ (22 nt);
H3

(SEQ ID NO: 108)

5′ TCTCGTGTCCCCCTGCTG 3′ (18 nt).

Each of the sequences above comprised a spacer at its 5′ end.

Spacer sequence 5′ GAATTCAAAGTTGCTGAGAATAGTTCAATGG AAGGAAGCG 3′ (SEQ ID NO: 36). Capture nucleotide sequences were aminated at their 5′ end.

### EXAMPLE 12

### Identification of G-Protein Serotonin Receptors Subtypes in Rat

Serotonin Receptor coupled to the G-protein were all amplified with the following primers:

Sense

Consensus for the subtypes 1A, 1B, 1C, 1D, 1E, 2A,
2B, 2C, 4, 6, 7: 5′ATCHTGCACCTSTGBGBCAT 3′ (SEQ ID
NO: 109) Tm = 58-64° C. (20 n6); H = C or A or T;
S = C or G; B = C or T or G;

(SEQ ID NO: 110)

1A ATCCTGCACCTGTGCGCCAT (0 mismatch) position
370-389;

(SEQ ID NO: 111)

1B ATCATGCATCTCTGTGTCAT (1 mismatch) position
397-416;

(SEQ ID NO: 112)

1C ATCATGCACCTCTGCGCCAT (0 mismatch) position
427-446;

(SEQ ID NO: 113)

1D ATCCTGCATCTCTGTGTCAT (1 mismatch) position
367-386;

(SEQ ID NO: 114)

1E ATCTTGCACCTGTCGGCTAT (2 mismatches) position
331-350;

(SEQ ID NO: 115)

2A ATCATGCACCTCTGCGCCAT (0 mismatch) position
487-506;

(SEQ ID NO: 116)

2B ATCATGCATCTCTGTGCCAT (1 mismatch) position
424-443;

(SEQ ID NO: 117)

2C ATCATGCACCTCTGCGCCAT (0 mismatch) position
24-43;

(SEQ ID NO: 118)

4 ATTTTTCACCTCTGCTGCAT (3 mismatches);

(SEQ ID NO: 119)

6 ATCCTCAACCTCTGCTTCAT (3 mismatches);

(SEQ ID NO: 120)

7 ATCATGACCCTGTGCGTGAT (3 mismatches);
Consensus 4, 6: 5′ ATCYTYCACCTCTGCYKCAT 3′ (SEQ ID
NO: 121) Tm = 52-64° C. (20 nt); K = G or T;
Y = T or C;

(SEQ ID NO: 122)

4 ATTTTTCACCTCTGCTGCAT (1 mismatch) position
322-341;

(SEQ ID NO: 123)

6 ATCCTCAACCTCTGCCTCAT (1 mismatch) position
340-359.
Consensus 5A, 5B: 5′ ATCTGGAAYGTGRCAGCCAT 3′ (SEQ
ID NO: 124) Tm = 58-62° C. (20 nt); Y = T or C;
R = A or G;

(SEQ ID NO: 125)

5A ATCTGGAATGTGACAGCAAT (1 mismatch) position
385-404;

(SEQ ID NO: 126)

5B ATCTGGAACGTGGCGGCCAT (1 mismatch) position
424-443.
Specific 7: 5′ ATCATGACCCTGTGCGTGAT 3′ (SEQ ID NO:
127) Tm = 56° C. (18 nt) position 517-536;
Specific 3B: 5′ CTTCCGGAACGATTAGAAA 3′ (SEQ ID NO:
128) TM = 54° C. (19 nt) position 404-422.

Antisense

Consensus for the subtypes 1A, 1B, 1C, 1D, 1E, 2A,
2B, 2C, 4, 7 Tm = 48-58° C.: 5′TTGGHNGCYTTCYGBTC
3′ (SEQ ID NO: 129); Y = T or C; H = A or T or C;
N = A or C or G or T; B = C or T or G;

(SEQ ID NO: 130)

1A TTCACCGTCTTCCTTTC (4 mismatches);

(SEQ ID NO: 131)

1B TTGGTGGCTTTGCGCTC (1 mismatch) position
913-929;

(SEQ ID NO: 132)

1C TTGGAAGCTTTCTTTTC (1 mismatch) position
922-938;

(SEQ ID NO: 133)

1D TTAGTGGCTTTCCTTTC (2 mismatches) position
877-893;

(SEQ ID NO: 134)

1E GTGGCTGCTTTGCGTTC (2 mismatches) position
862-878;

(SEQ ID NO: 135)

2A TTGCACGCCTTTTGCTC (2 mismatches) position
952-968;

(SEQ ID NO: 136)

2B TTTGAGGCTCTCTGTTC (2 mismatches) position
952-968;

(SEQ ID NO: 137)

2C TTGGAAGCTTTCTTTTC (1 mismatch) position
424-440;

(SEQ ID NO: 138)

4 TTGGCTGCTTTCCGGTC (2 mismatches);

(SEQ ID NO: 139)

7 GTGGCTGCTTTCTGTTC (1 mismatch) position
973-989.
Specific 1A: 5′ TTCACCGTCTTCCTTTC 3′ (SEQ ID NO:
140) Tm = 50° C. (17 nt) position 1018-1034.
Specific 4: 5′ TCTTGGCTGCTTTGGTC 3′ (SEQ ID NO:
141) Tm = 52° C. (17 nt) position 762-778.
Specific 6: 5′ ATAAAGAGCGGGTAGATG 3′ (SEQ ID NO:
142) Tm = 52° C. (18 nt) position 945-963.
Consensus 5A, 5B: 5′ CCTTCTGCTCCCTCCA 3′ (SEQ ID
NO: 143), Tm = 52° C. (16 nt);

(SEQ ID NO: 144)

5A CCTTCTGTTCCCTCCA (1 mismatch) position 823-840;

(SEQ ID NO: 145)

5B CCTTCTGCTCCCGCCA (1 mismatch) position 862-879.
Specific 3B: 5′ ACCGGGGACTCTGTGT 3′ (SEQ ID NO:
146) Tm = 52° C. (16 nt) position 1072-1089.

The following capture nucleotide sequences were chosen for the specific capture of the serotonin receptor subtypes sequences:

Capture nucleotide sequences
HTR1C

(SEQ ID NO: 147)

5′ CTATGCTCAATAGGATTACGT 3′ (21 nt);
HTR2A:

(SEQ ID NO: 148)

5′ GTGGTGAATGGGGTTCTGG 3′ (19 nt);
HTR2B:

(SEQ ID NO: 149)

5′ TGGCCTGAATTGGCTTTTTGA 3′ (21 nt);
HTR2C/1C:

(SEQ ID NO: 150)

5′ TTATTCACGAACACTTTGCTTT 3′ (22 nt);
HTR1B:

(SEQ ID NO: 151)

5′ AATAGTCCACCGCATCAGTG 3′ (20 nt);
HTR1D:

(SEQ ID NO: 152)

5′ GTACTCCAGGGCATCGGTG 3′ (19 nt);
HTR1A:

(SEQ ID NO: 153)

5′ CATAGTCTATAGGGTCGGTG 3′ (20 nt);
HTR1E:

(SEQ ID NO: 154)

5′ ATACTCGACTGCGTCTGTGA 3′ (20 nt);
HTR7:

(SEQ ID NO: 155)

5′ GTACGTGAGGGGTCTCGTG 3′ (19 nt);
HTR5A:

(SEQ ID NO: 156)

5′ GGCGCGTTATTGACCAGTA 3′ (19 nt);
HTR5B:

(SEQ ID NO: 157)

51 GGCGCGTGATAGTCCAGT 3′ (18 nt);
HTR3B:

(SEQ ID NO: 158)

51 GATATCAAAGGGGAAAGCGTA 3′ (21 nt);
HTR4:

(SEQ ID NO: 159)

5′ AAACCAAAGGTTGACAGCAG 3′ (20 nt);
HTR6:

(SEQ ID NO: 160)

5′ GTAGCGCAGCGGCGAGAG 3′ (18 nt).

Each of the sequences above comprises a spacer at its 5′ end

Spacer sequence 5′ GAATTCAAAGTTGCTGAGAATAGTTCAAT GGAAGGAAGCG 3′ (SEQ ID NO: 36). Capture nucleotide sequences were aminated at their 5′ end.

### EXAMPLE 13

### Identification of the HLA-A Subtypes

The HLA-A subtypes were amplified with the following consensus primers:

Sense

IPSCONA

(SEQ ID NO: 161)

5′ GACAGCGACGCCGCGAGCCA 3′

located at the position 181-200 of the gene, Tm 70° C.

Antisense

IPASCONA

(SEQ ID NO: 162)

5 CGTGTCCTGGGTCTGGTCCTCC 3′

located at the position 735-754 of the gene, Tm=74° C.

The size of the amplified product was 574 bp.

The following capture nucleotide sequences of 27 nucleotides were chosen for the specific capture of the HLA-A sequences:

Capture nucleotide sequences
HLA-A1
ITSA01:

(SEQ ID NO: 163)

5′ GGAGGGCCGGTGCGTGGACGGGCTCCG 3′;
HLA-A2
ITASA02:

(SEQ ID NO: 164)

5′ TCTCCCCGTCCCAATACTCCGGACCCT 3′;
HLA-A3
ITASA03A:

(SEQ ID NO: 165)

5′ CTGGGCCTTCACATTCCGTGTCTCCTG 3′;
ITSA03B:

(SEQ ID NO: 166)

5′ AGCGCAAGTGGGAGGCGGCCCATGAGG 3′;
HLA-A11
ITSA11A:

(SEQ ID NO: 167)

5′ GCCCATGCGGCGGAGCAGCAGAGAGCC 3′;
ITSA11B:

(SEQ ID NO: 168)

5′ CCTGGAGGGCCGGTGCGTGGAGTGGCT 3′;
HLA-A23
ITSA23A:

(SEQ ID NO: 169)

5′ GCCCGTGTGGCGGAGCAGTTGAGAGCC 3′;
ITASA23B:

(SEQ ID NO: 170)

5′ CCTTCACTTTCCCTGTCTCCTCGTCCC 3′;
HLA-A24
ITSA24A:

(SEQ ID NO: 171)

5′ GCCCATGTGGCGGAGCAGCAGAGAGCC 3′;
ITASA24B:

(SEQ ID NO: 172)

5′ TAGCGGAGCGCGATCCGCAGGTTCTCT 3′;
HLA-A25
ITASA25A

(SEQ ID NO: 173)

5′ TAGCGGAGCGCGATCCGCAGGCTCTCT 3′;
ITASA25B:

(SEQ ID NO: 174)

5′ TCACATTCCGTGTGTTCCGGTCCCAAT 3′;
HLA-A26
ITASA26:

(SEQ ID NO: 175)

5′ GGGTCCCCAGGTTCGCTCGGTCAGTCT 3′;
HLA-A29
ITASA29:

(SEQ ID NO: 176)

5′ TCACATTCCGTGTCTGCAGGTCCCAAT 3′;
HLA-A30
ITASA30:

(SEQ ID NO: 177)

5′ CGTAGGCGTGCTGTTCATACCCGCGGA 3′;
HLA-A31
ITASA31:

(SEQ ID NO: 178)

5′ CCCAATACTCAGGCCTCTCCTGCTCTA 3′;
HLA-A33
ITSA33:

(SEQ ID NO: 179)

5′ CGCACGGACCCCCCCAGGACGCATATG 3′;
HLA-A68
ITSA68A:

(SEQ ID NO: 180)

5′ GGCGGCCCATGTGGCGGAGCAGTGGAG 3′;
ITASA68B:

(SEQ ID NO: 181)

5′ GTCGTAGGCGTCCTGCCGGTACCCGCG 3′;
HLA-A69
ITASA69:

(SEQ ID NO: 182)

5′ ATCCTCTGGACGGTGTGAGAACCGGCC 3′.

Each of the sequences above comprised an aminated spacer at its 5′ end. Spacer sequence 5′ GAATTCAAAGTTGCTGAGAATAGTTCAATGGAAGGAAGCG 3′ (SEQ ID NO: 36).

### EXAMPLE 14

### Identification of Cytochrome P450 3a Forms

The Cytochrome P450 forms were amplified with the following consensus primers:

Sense

Consensus: 5′ GCCAGAGCCTGAGGA 3′ (SEQ ID NO: 183) located at the position 1297-1311 of the 3a3 gene, Tm=50° C.

Antisense

Consensus a3, a23, a1, a2: 5′ TCAAAAGAAATTAACAGAGA 3′ (SEQ ID NO: 184) located at the position 1839-1858 of the 3a3 gene, Tm=50° C.

Specific a9: 5′ ACAATGAAGGTAACATAGG 3′ (SEQ ID NO: 185) located at the position 2015-2033 of the 3a9 gene Tm=52° C.

Specific a18: 5′ ACTGATGGAACTAACTGG 3′ (SEQ ID NO: 186) located at the position 1830-1846 of the 3a18 gene Tm=52° C.

The length of the PCR product was around 560 bp.

The following capture nucleotide sequences were chosen for the specific capture of the cytochrome P-450 3a sequences:

Capture nucleotide sequence
3a1

(SEQ ID NO: 187)

5′ TGTTTTGATTCGGTACATCTTTG 3′ (23 nt);
3a3

(SEQ ID NO: 188)

5′ TTGATTTGGTACATCTTTGCT 3′ (21 nt);
3A9

(SEQ ID NO: 189)

5′ ACTCCTGGGGGTTTTGGGTG 3′ (20 nt);
3A18

(SEQ ID NO: 190)

5′ ATTACTGAGTATTCAGAAATTCAC 3′ (24 nt);
3A2

(SEQ ID NO: 191)

5′ GGTTAAAGATTTGGTACATTTATGG 3′ (25 nt).

Each of the sequences above comprised a spacer at its 5′ end

Spacer sequence 5′ GAATTCAAAGTTGCTGAGAATAGTTCAAT GGAAGGAAGCG 3′ (SEQ ID NO: 36). Capture nucleotide sequences were aminated at their 5′ end.

Each of the sequences above comprises a spacer at its 5′ end.

Spacer sequence 5′ GAATTCAAAGTTGCTGAGAATAGTTCAAT GGAAGGAAGCG (SEQ ID NO: 36).

### EXAMPLE 15

### Identification of GMO on Biochips

The following primers were chosen for the amplification step of the GMO.

Consensus primers to detect GMO on biochips:

| | |
|---|---|
| Forward | Reverse |

| | |
|---|---|
| OPP35S1 (P-35S) | OPT352 (T-35S) |
| 5′CGTCTTCAAAGCAAGTGGATTG3′ | 5′GAAACCCTAATTCCCTTATCAG |
| (SEQ ID NO: 192) | GG3′ |
| | (SEQ ID NO: 193) |
| OPTE91 (T-E9) | OPTnos2 (T-nos) |
| 5′TCATGGATTTGTAGTTGAGTATG | 5′ATCTTAAGAAACTTTATTGCCA |
| AA3′ | AATGT3′ |
| (SEQ ID NO: 194) | (SEQ ID NO: 195) |
| OPEPS3 (EPSPS) | OPTE92 (T-E9) |
| 5′GCTGTAGTTGTTGGCTGTGGT3′ | 5′CTGATGCATTGAACTTGACG |
| (SEQ ID NO: 196) | A3′ |
| | (SEQ ID NO: 197) |
| OPLB1 | OPEPS4 (EPSPS) |
| (octopine Left Border) | 5′GCGACATCAGGCATCTTGTT3′ |
| 5′ATCAGCAATGAGTATGATGGTCA | (SEQ ID NO: 199) |
| AT3′ | |
| (SEQ ID NO: 198) | |
| OPLB3 | OPRB2 |
| (nopaline Left Border) | (octopine Right Border) |
| 5′ACAAATTGACGCTTAGACAACT3′ | 5′TGCCAGTCAGCATCATCACA |
| (SEQ ID NO: 200) | C3′ |
| | (SEQ ID NO: 201) |
| | OPRB4 |
| | (nopaline Right Border) |
| | 5′TAAGGGAGTCACGTTATGAC |
| | C3′ |
| | (SEQ ID NO: 202) |

These primers allowed the amplification of the following genes:

1) CTP1, CTP2, CP4EPSPS, S CryIAb and hsp 70 Int. in Mon 809 (corn, Monsanto);

2) hsp 70 Int. and S CryIAb in Mon 810 (corn, Monsanto);

3) S CryIAb and S Pat in Bt 11 (corn, Novartis);

4) CTP4 and EPSPS in GTS40-3-2 (soybean, Monsanto).

The capture nucleotide sequences were chosen in these sequences to allow discrimination. Each of the sequences above comprised a spacer at its 5′ end.

Spacer sequence

(SEQ ID NO: 36)

5′GAATTCAAAGTTGCTGAGAATAGTTCAATGGAAGGAAGCG.

The following sequences were chosen as specific capture probes of the GMO:

OT1 pat (T25, Bt11)

| | |
|---|---|
| TGGTGGATGGCATGATGTTGGTTTTTGGCA; | (SEQ ID NO: 203) |
| OT2 CryIAb (Bt11) | |
| GCACGAAGCTCTGCAATCGCACAAACCCGT; | (SEQ ID NO: 204) |
| OT3 P-PCK (Bt176) | |
| TGGGGGTAGCTGTAGTCGGACTCGGACTGG; | (SEQ ID NO: 205) |
| OT4 CP4EPSPS/Tnos | |
| AGCCCCTAGCTAGGGGGTGGCCAGGAAGTA. | (SEQ ID NO: 206) |

### EXAMPLE 16

### Detection of Gyrase (Sub-Unit A) Sequences on Array Bearing Genus and Species Specific Capture Nucleotide Sequences Example of Bacterial Detection

Amplification of the Sequences

The amplified target sequences are fragments of the gyrase gene (sub-unit A) sequences corresponding to the different genus and species (table 1) which were amplified by a PCR using the following consensus primers:

| | |
|---|---|
| Pgyr1: 5′GANGTNATSGGTAAATAYCA 3′; | (SEQ ID NO: 207) |
| Pgyr2: 5′CGNRYYTCVGTRTAACG 3′. | (SEQ ID NO: 208) |

The PCR was performed in a final volume of 100 μl containing: 3 mM MgCl₂, 1 mM Tris pH 8, 1 μM of each primer, 200 μM of dATP, dCTP and dGTP, 150 μM of dTTP, 50 μM of biotin-16-dUTP, 2.5 U of Taq DNA polymerase (Boehringer Mannheim, Allemagne), 1 U of Uracil-DNA-glycosylase heat labile (Boehringer Mannheim, Allemagne), 1 ng of plasmid containing gyrase gene. Samples were first denatured at 94° C. for 5 min. Then 40 cycles of amplification were performed consisting of 30 sec at 94° C., 45 sec at 48° C. and 30 sec at 72° C. and a final extension step of 10 min at 72° C. Water controls were used as negative controls of the amplification. The sizes of the amplicons obtained using these primers were 166 bp for all genera.

Production of the Capture Nucleotide Sequences and of the Targets

The capture nucleotide sequences contain a spacer fixed on the support by its 5′ end and of the following sequence

(SEQ ID NO: 209)

5′ATAAAAAAGTGGGTCTTAGAAATAAATTTCGAAGTGCAATAATTATTA
TTCACAACATTTCGATTTTTGCAACTACTTCAGTTCACTCCA3′),

followed by the following specific sequences for the various Gyrase from the different bacteria:

| Name | |
|---|---|
| Capture | |
| nucleotide | |
| sequence | Sequence (5′ -> 3′) |
| A. Genus level | |
| T. Staphy genus | GACTCWTCAATTTATGAWGCHATGGTAHGAAY |
| | GG |
| | (SEQ ID NO: 210) |
| T. Entero genus | GACAGTGCGATYTAYGARTCAATGGTRCGG |
| | (SEQ ID NO: 211) |
| T. Strepto genus | TGGTTCGTATGGCTCAATGGTGGAGYTAY |
| | (SEQ ID NO: 212) |
| B. Species level | |
| T S. aureus | CTCAAGATTTCAGTTATCGTTATCCGCT |
| | (SEQ ID NO: 213) |
| T S. epidermidis | CCCAAGACTTTAGTTATCGTTATCCACT |
| | (SEQ ID NO: 214) |
| T S. hominis | CACAAACCTTTAGCTATCGTTATCCTC |
| | (SEQ ID NO: 215) |
| T Entero. faecium | ACAGCCATTCAGCTACCGTTATATGCT |
| | (SEQ ID NO: 216) |
| T Entero. faecalis | AACCTTTTAGTTATCGGGCTATGTTAGTT |
| | (SEQ ID NO: 217) |
| T S. pneumoniae | GATGGAGATAGTGCTGCCGCTCAAC |
| | (SEQ ID NO: 218) |
| T S. epyogenes | CTTGTTGATGGGCATGGCAATTTTGG |
| | (SEQ ID NO: 219) |
| T H. influenzae | TTCTCACTTCGCTATATGTTGGTTGATG |
| | (SEQ ID NO: 220) |

The capture nucleotide sequences were first synthesized chemically and later on produced by PCR amplification after cloning of the sequences into the plasmid pGEM-T Easy Vector System (Promega, Madison, USA). The capture nucleotide sequences were then produced by amplification of the plasmids using a common 5′ aminated primer 5′ GAATTCAAAGTTGCTGAGAATAGTTCA (SEQ ID NO: 221) and a second primer of 27 bases complementary of each capture nucleotide sequence.

The aminated capture polynucleotide sequences (longer than 100 bases) were spotted from solutions at concentrations ranging from 150 to 3000 nM. The capture nucleotide sequences were printed onto the aldehyde microscopic slides with a home made robotic device (250 μm pins from Genetix (UK). The solutions of spotting were from AAT (Namur, Belgium). The spots have 400 μm in diameter and the volume dispensed is about 0.5 nl. Slides are dried at room temperature and stored at 4° C. until used.

Hybridization

At 65 μl of hybridization solution (AAT, Namur, Belgium) were added 5 μl of amplicons and the solution was loaded on the array framed by a hybridization chamber. For positive controls 2 nM biotinylated CMV amplicons of 437 bp were added to the solution; their corresponding capture nucleotide sequences were spotted on the array. The chamber was closed with a coverslip and slides were denatured at 95° C. for 5 min. The hybridization was carried out at 650 for 30 min. Samples were then washed 4 times with a washing buffer.

Colorimetric Detection

The glass samples were incubated 45 min at room temperature with 800 μl of streptavidin labeled with colloidal gold 1000× diluted in blocking buffer (Maleic buffer 100 mM pH 7.5, NaCl 150 mM, Gloria milk powder 0.1%). After 5 washes with washing buffer, the presence of gold served for catalysis of silver reduction using a staining solution (Silver Blue Solution, AAT, Namur, Belgium). The slides were incubated 10 min with 800 μl of revelation mixture, then rinsed with water, dried and analyzed using a microarray reader (Worstation, AAT, Namur, Belgium). The spots of the arrays were then quantified by a specific quantification software.

### EXAMPLE 17

### Detection of Virus Species and Subtypes

The virus to be detected was the adenovirus, the herpes virus 1, 5 and 4. The consensus primers for the virus amplification were

(SEQ ID NO: 222)

A(G)C(A,T)G(C,T)GCCGCCGTGT(A)T(A,C)C(T)G(A,C)
and

(SEQ ID NO: 223)

GT(G,C)G(T,A)GTTGTTTTTG(A)T(C)G(C)G(T).

The amplicons of the virus are respectively of 315, 331, 779, and 820 bases long for the 4 virus corresponding to the sequences N°420-734, 7924-8254, 1562-2340, 120761-130580.

The conditions for the PCR amplification were as described in example 1 but with an annealing temperature of 45° C. After amplification, the amplicons were hybridized on an array bearing the capture nucleotide sequences for each virus species and subtypes. The capture nucleotide sequences were composed of a spacer fixed by its 5′ end to the slides and have the sequence as in example 16 and a specific part located on the 3′ end of the capture nucleotide sequence.

Specific sequences of the capture nucleotide sequences:

| | |
|---|---|
| Adenovirus: | |
| 5′-AACTCTTCTCGCTGGCACTCAAGAGTG-3′; | (SEQ ID NO: 224) |
| Herpes virus 1: | |
| 5′-GTGGAAGTCCTGATACCCATCCTACAC-3′; | (SEQ ID NO: 225) |
| Herpes virus 5: | |
| 5′-AAAAGCGTGTGATCTGACCGAGGCGAA-3′; | (SEQ ID NO: 226) |
| Herpes virus 4: | |
| 5′-AGGTCCTTGAGGAAGAAGTGTTCCAGG-3′; | (SEQ ID NO: 227) |
| Tm = 82° C. | |

The hybridization, the colorimetry labeling and the quantification were performed as in example 1.

### EXAMPLE 18

### Detection of Cytochrome b Sequences on Array Bearing Species Specific Capture Nucleotide Sequences. Example of Meat Origin

The amplified target sequences are fragments of the cytochrome b gene sequences corresponding to the different species were amplified by a PCR using the following consensus primers:

| | |
|---|---|
| Meat1 5′TCCTCCCATGAGGAGAAATAT 3′; | (SEQ ID NO: 228) |
| Meat2 5′AGCGAAGAATCGGGTAAGGGT 3′. | (SEQ ID NO: 229) |

The PCR were performed as in example 1. The sizes of the amplicons obtained using these primers were between 130 and 147 bp for all genus. After amplification, the amplicons were hybridized on an array bearing the capture nucleotide sequences for each species. The capture nucleotide sequences were composed of a spacer fixed by its 5′ end to the slides and having the same sequence as in example 1 and a specific part located on the 3′ end of the capture nucleotide sequence.

Spacer

(SEQ ID NO: 209)

5′ATAAAAAAGTGGGTCTTAGAAATAAATTTCGAAGTGCAATAATTATTA
TTCACAACATTTCGATTTTTGCAACTACTTCAGTTCACTCCA3′

Specific sequences of the capture nucleotide sequences:

Chicken

(SEQ ID NO: 230)

CCTTAACGACTCTTATCCAAACACTATGCCACCGGGGAG;
Duck

(SEQ ID NO: 231)

CCCTAACGACTCTTATCCAAACACTACTGCCATCGGGGAG;
Ostrich

(SEQ ID NO: 232)

CCTTAACGAACTCTAAG;
Pig

(SEQ ID NO: 233)

AAAGAGGAGTAGAATCACGATTAAG;
Quail

(SEQ ID NO: 234)

CCATGTCGACTCTTATCCAAACACTACTGCCATCGTGGAG;
Rabbit

(SEQ ID NO: 235)

CCCTAACGACTATCCTCCAATCACTAATGCCAACGAGGGG;
Turkey

(SEQ ID NO: 236)

CCCTAACGACTCTTATCCAAACACTACTGCCATCGGGAG;
Wild pig

(SEQ ID NO: 237)

CCCTATCGACTATCTTCTAAACACTACTGGCATCGAGGAG;
Cow

(SEQ ID NO: 238)

CCTAACGACTATTCTCCAACCACTACTGACAACGAGGAG.

The consensus capture nucleotide sequence for all these animal detection

(SEQ ID NO: 239)

ATTCTGAGGGGCACCGTCATCACAAACCTATTTCAGCAATCCCCTACATG
GCAAACCCTAGTAGAATGAGCCTGAGGGGGATTTTCAGTGACAACC

To identify the cow species, another couple of consensus primer was designed:

Cow1

(SEQ ID NO: 240)

AAGACATAATATGTATATAGTAC;
Cow2

(SEQ ID NO: 241)

GAAAAATTTAAATAAGTATCTAG.

Specific capture nucleotide sequences have been designed:

BrownSwiss

(SEQ ID NO: 242)

GCGGCATGATAATTA;
Jersey

(SEQ ID NO: 243)

CGCTATTCAATGAAT;
Ayrshire

(SEQ ID NO: 244)

GCTCACCATAACTGT;
Hereford

(SEQ ID NO: 245)

ATCTGATGGTAAGGA;
Simmental

(SEQ ID NO: 246)

ATAAGCCTGGACATT;
Piemontaise

(SEQ ID NO: 247)

ATAAGCATGGACATT;
Canadienne

(SEQ ID NO: 248)

TCACTCGGCATGATA;
RedAngus

(SEQ ID NO: 249)

AATGGTAGGGGATAT;
Limousine

(SEQ ID NO: 250)

ATGGACTCATGGCTA;
AberdeenAngus

(SEQ ID NO: 251)

TATTCAATGAACTTT;
Butana

(SEQ ID NO: 252)

GCATGGGGTATATAA;
Charolais

(SEQ ID NO: 253)

ATAAGCGTGGACATTA;
Fresian

(SEQ ID NO: 254)

CCTTAAATACCTACC;
Kenana

(SEQ ID NO: 255)

TGCTATAGAAGTCAT;
N′Dama

(SEQ ID NO: 256)

TGTTATAGAAGTCAT.

The hybridization, the colorimetry labeling and the quantification were performed as in example 1.

### EXAMPLE 19

### Detection of Sucrose Synthase Sequences on Array Bearing Species Specific Capture Nucleotide Sequences Example of Plant Origin

The amplified targets are fragments of the sucrose synthase gene sequences corresponding to the different species were amplified by a PCR using the following consensus primers:

PPss3

(SEQ ID NO: 257)

5′ GGTTTGGAGARRGGNTGGGG 3′;
PPss4

(SEQ ID NO: 258)

5′ TCCAADATGTAVACAACCTG 3′.

The PCR were performed as in example 1. The sizes of the amplicons obtained using these primers were 221 bp for all genuses. After amplification, the amplicons were hybridized on an array bearing the capture nucleotide sequences for each species. The capture nucleotide sequences were composed of a spacer fixed by its 5′ end to the slides and having the following sequence and a specific part located on the 3′ end of the capture nucleotide sequence.

Spacer

(SEQ ID NO: 209)

5′ATAAAAAAGTGGGTCTTAGAAATAAATTTCGAAGTGCAATAATTATTA
TTCACAACATTTCGATTTTTGCAACTACTTCAGTTCACTCCA3′.

Specific sequences of the capture nucleotide sequences:

TPss1 (potato)

(SEQ ID NO: 259)

GAAGCATGCATACCATCTCTAGCA;
TPss3 (tomato)

(SEQ ID NO: 260)

GGAGCATGCAGATCATCTCTAGAA;
TPss7 (oryza)

(SEQ ID NO: 261)

GAAGCAAGTGGATGGTGTCAAGCA;
TPss8 (zea)

(SEQ ID NO: 262)

AGAGGAGGTGGATAGTCTCCTGTG;
TPss9 (soja)

(SEQ ID NO: 263)

AGAGAAGTTGAATTGACTCAAGGA;
TPss11 (wheat)

(SEQ ID NO: 264)

AGAGAAGGTGGATAGTCTCGCTCG;
TPss12 (barley)

(SEQ ID NO: 265)

AGAGAAGGTGGATAGTCTCGCTCG;
TPss13 (bean)

(SEQ ID NO: 266)

ATAGAAGCTGAATGGACTCGAGCA;
TPss14 (carrot)

(SEQ ID NO: 267)

GAAGCATGTGAAACATCTCAGTAA.

The hybridization, the colorimetry labeling and the quantification were performed as in example 1.

### EXAMPLE 20

### Detection of Cytochrome b Sequences on Array Bearing Species Specific Capture Nucleotide Sequences Example of Fish Species, Genus and Families

The amplified target sequences are fragments of the cytochrome b gene sequences corresponding to the different species were amplified by a PCR using the following consensus primers:

Fish1

(SEQ ID NO: 268)

5′ ACTATTHCTAGCCATVCAYTA 3′;
Fish2

(SEQ ID NO: 269)

5′ AGGTAGGAGCCATAAAGACCTCG 3′.

The PCR were performed as in example 1. The sizes of the amplicons obtained using these primers were 170 bp for all genuses. After amplification, the amplicons were hybridized on an array bearing the capture nucleotide sequences for each species. The capture nucleotide sequences were composed of a spacer fixed by its 5′ end to the slides and having the following sequence and a specific part located on the 3′ end of the capture nucleotide sequence.

Spacer

(SEQ ID NO: 209)

5′ATAAAAAAGTGGGTCTTAGAAATAAATTTCGAAGTGCAATAATTATTA
TTCACAACATTTCGATTTTTGCAACTACTTCAGTTCACTCCA3′

Specific sequences of the capture nucleotide sequences for the species:

G. morhua:

(SEQ ID NO: 270)

AAGGCTTAATCAGTCGGCATCAAATGTA;
G. macrocephalus:

(SEQ ID NO: 271)

AAGGCTTACTCAGTTGGCATTAAATGTA;
P. flesus:

(SEQ ID NO: 272)

GAAGCCTACTCAGTTGGCATCAACTGCA;
M. merluccius:

(SEQ ID NO: 273)

AACGCCTAATCAGTAGGCATTAAATGCA;
O. mykiss:

(SEQ ID NO: 274)

AAAGCTTACTCAGTCGGCATTGATTGTA;
P. platessa:

(SEQ ID NO: 275)

GAAGCCTATTCAGTCGGCATCAACTGCA;
P. virens:

(SEQ ID NO: 276)

AAAGCTTAATTAGTCGGCATTAAATGTA;
S. salar:

(SEQ ID NO: 277)

CAATGCCTACTCAGTCGGTATCGATTGTA;
S. pilchardus:

(SEQ ID NO: 278)

GAAGCTTAGTCAGTAGGCATCAAATGCA;
A. thazard:

(SEQ ID NO: 279)

AAAGCCTATTCAGTTGGCTTCAAATGTA;
T. alalunga:

(SEQ ID NO: 280)

AAAGCCTACTCAGTAGGCTTCAAATGTA;
T. obesus:

(SEQ ID NO: 281)

AAAGCCTACTCAGTTGGCTTTAACTGTTA;
R. hippoglossoides:

(SEQ ID NO: 282)

GAAGCCTATTCAGTCGGCATCAACTGCA;
S. trutta:

(SEQ ID NO: 283)

AAAGCCTACTCAGTCGGCATCGATTGCA;
S. sarda:

(SEQ ID NO: 284)

AAAGCCTAATCAGTCGGCTTTAATTGCA;
T. thynnus:

(SEQ ID NO: 285)

AAGGCCTATTCAGTTGGCTTCAACTGTA;
S. scombrus:

(SEQ ID NO: 286)

AACGCCTACTCAGTAGGCTTCAAATGCA.

Specific sequences of the capture nucleotide sequences for the families:

Salmonidae:

(SEQ ID NO: 287)

AAACATTCACGCTAACGGAGCATCTTTCTTCTTTA TCTGT;
Pleuronectidae:

(SEQ ID NO: 288)

AAGCATTCATGCCAACGGCGCATCATTCTTTTT CATTTGC;
Pleuronectidae:

(SEQ ID NO: 289)

GAATATACATGCTAATGGTGCCTCTTTCTTTTTTATTTGT;
Scombridae:

(SEQ ID NO: 290)

AAACCTCCACGCAAACGGAGCCTCTTTCTTTCTTTATCTGC.

Among this family, a consensus capture nucleotide sequence was designed to detect the Thunnus genus: ATTCCACATCGGCCG (SEQ ID NO: 291)

Consensus capture nucleotide sequences for these various fish families:

(SEQ ID NO: 292)

ATCCGAAACATCCACGCAACGGGCATCTTTCTTCTTTATCTGTATCTACT
TACACAT

The hybridization, the colorimetry labeling and the quantification were performed as in example 1.

### EXAMPLE 21

### Detection of Cytochrome P450 Isoforms After Amplification with Consensus primers and Hybridization of the Amplicons on Arrays

The amplified targets are fragments of the cytochrome P450 gene sequences corresponding to the different families which were amplified by a PCR using the following consensus primers:

p450-1

(SEQ ID NO: 293)

5′TCCGCAACTTGGGCCTGGGCAAGA 3′;
p450-2

(SEQ ID NO: 294)

5′CCTTCTCCATCTCTGCCAGGAAG 3′.

The conditions for the PCR amplification are the same as in example 1. The sizes of the amplicons obtained using these primers were 970 bp. After amplification, the amplicons were hybridized on an array bearing the capture nucleotide sequences for each single point mutation.

The capture nucleotide sequences were composed of a spacer fixed by its 5′ end to the slides and having the following sequence and a specific part located on the 3′ end of the capture nucleotide sequence.

Spacer

(SEQ ID NO: 36)

5′ GAATTCAAAGTTGCTGAGAATAGTTCAATGGAAGGAAGCG 3′

Specific sequences of the capture nucleotide sequences for the single point mutations from different families of cytochrome p450.

Target Gene: Human CYP2D6

| | |
|---|---|
| Name | Sequence (5′-3′) |

| | | |
|---|---|---|
| WT | | GAAAGGGGCGTCCTGGG |
| | | (SEQ ID NO: 295) |
| *4 | substitution T in C | GAAAGGGGCGTCtTGGG |
| | at position 13 of WT | (SEQ ID NO: 296) |
| WT | | GCTAACTGAGCACAGGA |
| | | (SEQ ID NO: 297) |
| *3 | Deletion of A at | GCTAACTGAGCACGGA |
| | position 14 of WT | (SEQ ID NO: 298) |
| WT | | CTCGGTCACCCCCTGC |
| | | (SEQ ID NO: 299) |
| *6 | Deletion of C at | CTCGGTCACCCCTGC |
| | position 12 of WT | (SEQ ID NO: 300) |

Target Gene: Human CYP2C19

| | |
|---|---|
| Name | Sequence (5′-3′) |

| | | |
|---|---|---|
| WT | | AATTATTTCCCAGGAA |
| | | (SEQ ID NO: 301) |
| *2 | substitution G in A | AATTATTTCCCaGGAA |
| | | (SEQ ID NO: 302) |
| WT | | AGCACCCCCTGAATCC |
| | | (SEQ ID NO: 303) |
| *3 | substitution G in A | AGCACCCCCTGaATCC |
| | | (SEQ ID NO: 304) |

The hybridization, the colorimetry labeling and the quantification were performed as in example 1.

### EXAMPLE 22

### Evidence for Bacterial Presence During the PCR (Real Time PCR) and Identification on Microarrays

Example of detection of the main bacteria responsible for meningitis by real-time PCR on cerebrospinal fluid was combined with genus and species sequence identification on DNA microarray

The tuf is phylogenetically well conserved gene amongst bacteria, it encodes an elongation factor (TE). The biological sample for the detection of meningitis was cerebrospinal fluid. Indeed, this medium is normally sterile and if there is an infection, it would be contaminated by only one pathogen. Thus it limits the risk to amplify other genus with consensus primers.

For a real-time PCR consensus primers for the tuf gene, amplify all genus and species of interest and the consensus probe for the tuf gene was labeled with two fluorochromes (quencher and emitter) as internal control of the PCR.

Biochips bearing specific capture probes for bacteria genus and species currently found in meningitis infections were:

Neisseria menengitidis serogroup A;

Neisseria menengitidis serogroup B;

Haemophylus influenzae;

Escherichia coli;

Streptococcus pneumoniae;

Streptococcus agalactiae;

Staphylococcus aureus;

Staphylococcus epidermidis;

Staphylococcus haemolyticus;

Staphylococcus hominis.

Staphylococcus saprophyticus

For the Primers Consensus Sense were: 5′ GAATTRGTTGAAATGGAA 3′ 18 NT (SEQ ID NO: 305); (R=A or G) position 443-460 Tm=46-48° C., 1 mismatch maximum.

For the Consensus Antisense were: 5′ GTAGTACGGAARTAGAA 3′ 17 nt (SEQ ID NO: 306), (R=A or G), position 995-1011 Tm=46-48° C., 1 mismatch maximum.

For the Double labeled Probe (sense) were: 5′ GGTGTTGAAATGTTCC 3′ 16 nt (SEQ ID NO: 307) position 776-792 Tm=46° C., 1mismatch maximum

Size of the amplified product: 569 bp.

Genus Specific Capture Probes

1) Meningococcus

(SEQ ID NO: 308)

5′ CGACCTGCTGTCCAGCT 3′ (17 nt).

Identical for serogroup A and B and a minimum of 5 mismatches against the other genus.

2) Streptococcus

(SEQ ID NO: 309)

5′ CTTCAGGACGTATCGACC 3′ (18 nt)

Identical for Streptococcus pneumoniae and Streptococcus agalactiae and a minimum of 5 mismatches against the other genus.

3) Staphylococcus

(SEQ ID NO: 310)

5′ TTATTAGACTACGCTGAAG 3′ (19 nt)

Identical for Staphylococcus aureus, Staphylococcus epidermidis, Staphylococcus haemolyticus, Staphylococcus hominis, Staphylococcus saprophyticus and a minimum of 6 mismatches against the other genus.

Species specific capture probes
1) Neisseria menengitidis serogroup A:

(SEQ ID NO: 311)

5′ TCTATTTCCGGTCGTGGT3′ (18 nt);
2) Neisseria menengitidis serogroup B:

(SEQ ID NO: 312)

′ CCATTTCCGGCCGCGG3′ (16 nt);
3) Hoemophylus influenzae:

(SEQ ID NO: 313)

5′ GAGTTAGCAAACCACTTAG3′ (19 nt);
4) Escherichia coli:

(SEQ ID NO: 314)

5′ AACTGGCTGGCTTCCTG3′ (17 nt);
5) Streptococcus pneumoniae:

(SEQ ID NO: 315)

5′ GTATCAAAGAAGAAACTCAAA3′ (21 nt);
6) Streptococcus agalactiae:

(SEQ ID NO: 316)

5′ GTATTAAAGAAGATATCCAAA3′ (21 nt)
7) Staphylococcus aureus:

(SEQ ID NO: 317)

5′ GGTTTACATGACACATCTAA3′ (20 nt)
8) Staphylococcus epidermidis:

(SEQ ID NO: 318)

5′ GTATGCACGAAACTTCTAAA3′ (20 nt)
9) Staphylococcus haemolyticus:

(SEQ ID NO: 319)

5′ GTATCCATGACACTTCTAAA3′ (20 nt)
10) Staphylococcus hominis:

(SEQ ID NO: 320)

5′ GGTATCAAAGAAACTTCTAAA3′ (21 nt)
11) Staphylococcus saprophyticus:

(SEQ ID NO: 321)

5′ ATGCAAGAAGAATCAAGCAA3′ (20 nt)

Each of the sequences above comprised a spacer at its 5′ end Spacer sequence

(SEQ ID NO: 36)

5′ GAATTCAAAGTTGCTGAGAATAGTTCAATGGAAGGAAGCG 3′.

Capture probes were aminated at their 5′ end.

### EXAMPLE 23

### HLA Identification

Glass surface was activated in order to bear aldehyde groups as proposed by EP-00870184.9. The slides were then incubated with a Protein A at 5 μg/ml in PBS solution for 60 min. The slides were washed in PBS and then incubated for 5 min. in NaBH₄ solution at 2.5 mg/ml. After washing they were incubated for 2 h with 10% milk powder and then washed again. Antibodies at concentration of 0.1 mg/ml were spotted on the glass slides with solid pins of 0.250 mm diameter and the spots were around 0.35 mm diameter final. The spotting solution contained buffer borate 0.05 M pH 8, glycerol 40% and NP40 0.02%. After 3 washes with 0.01 M phosphate pH 7.4, non-specific binding sites were blocked with PBS containing milk powder at 0.1% for 1 h at 20° C.

For the reaction of the targets, the slides were incubated for 1 h at 20° C. with the samples in the presence of PBS containing milk powder at 0.1%. After 4 washes of one minute with a 10 mM maleate buffer containing 15 mM NaCl (washing buffer) the slides were incubated for 45 min. at 20° C. with an antibody common for the various targets potentially present in the samples, then with a conjugate of anti-IgG/gold particles of 10 nm diameter (diluted 100 times) in 100 mM maleate buffer containing 150 mM NaCl.

The slides were washed 5 times in the same washing buffer as before and then incubated for 10 min. in the Silver Blue detection solution (AAT Namur) for obtaining the silver crystal precipitation. The slides were finally washed in water before being read in the Silver Blue Reader (AAT).

The HLA-A typing was obtained using antibodies specific of the types or subtypes. The antibodies against HLA-ABC common, HLA-B7, HLA-B27, were obtained from Cymbus Biotechnology, Ltd., Hampshire, UK. Other antibodies were from Pel-Freez especially the antibodies directed against the HLA-A2, A₂O₃ and A210 or HLA-B39, B3901, B3902, which allow typing and subtyping of the HLA. Lymphocytes were isolated from the blood according to the classical microlypophocytotoxicity assay (Pel-Freez, Brown Deer, Wis., USA). Lymphocytes at 10×10⁶ cells/ml were incubated for 30 min. at 37° C. with the antibody array in RPMI 1640 media with Hepes buffer. The arrays are then washed 4 times in the same medium. The second antibodies for cells were directed against CD-2 and CD-19. Then the anti-IgG/nano-gold complexes were incubated followed by the Silver Blue (AAT, Namur, Belgium) for the detection.

### EXAMPLE 24

### Amplification and Detection of 3 Different GMO (Genetically Modified Organisms) and 1 Plant Species

The list of the four targeted GMO and the targeted plant species (invertase for Maize) to be detectable in the assay is presented in the table below together with the two primers used for each of the gene sequences to be amplified. Two common sequences are present on the two primers of the same pair. These common sequences which are later used as universal amplifying sequences are in bold. Specific sequences are in italics.

| | | |
|---|---|---|
| Target | | |
| GMO | Primer forward | Primer reverse |

| | | |
|---|---|---|
| T25 | TCTATATGCTCCACAGTAT | ACTATATGCTCCACTCTAT |
| | GCGAACAAGCGTGTCGTGC | GCCTCGACATGATACTCCT |
| | TCCA | TCCAC |
| | (SEQ ID NO: 322) | (SEQ ID NO: 323) |
| T45 | TCTATATGCTCCACAGTAT | ACTATATGCTCCACTCTAT |
| | GCGACAATGGACACATGAA | GCCTGACTCTGTATGAACT |
| | TTATG | GTTCG |
| | (SEQ ID NO: 324) | (SEQ ID NO: 325) |
| MON531 | TCTATATGCTCCACAGTAT | ACTATATGCTCCACTCTAT |
| | GCGATTTCCCATTCGAGTT | GCCTAACCAATGCCACCCC |
| | TCTCAC | ACTG |
| | (SEQ ID NO: 326) | (SEQ ID NO: 327) |
| LLRICE62 | TCTATATGCTCCACAGTAT | ACTATATGCTCCACTCTAT |
| | GCGACTGGCGTAATAGCGA | GCCTCTAACGGGTGCATCG |
| | AGAG | TCTA |
| | (SEQ ID NO: 328) | (SEQ ID NO: 329) |
| Invertase | TCTATATGCTCCACAGTAT | ACTATATGCTCCACTCTAT |
| | GCGACGCTCTGTACAAGCG | GCCTCAAAGTGTTGTGCTT |
| | TGC | GGACC |
| | (SEQ ID NO: 330) | (SEQ ID NO: 331) |

Amplifications were carried out in 25 μl volume reactions, with 100 ng of genomic DNA (each GMO was diluted in 100% non-GMO maize DNA to a final concentration of 0.1%). The GMO DNAs were obtained from the American Oil Chemist Society (AOCS) (Boulder, Urbana, Ill., USA). The sample contains the DNA of T25, Mon531 and LLRice62 at 0.1% in 99.7% DNA of non-GMO maize. The PCR reaction contained both the specific and the universal primers. The universal primers were TCTATATGCTCCACAGTATGCGA (Universal Primer A) (SEQ ID NO: 332) and ACTATATGCTCCACTCTATGCCT (Universal Primer B) (SEQ ID NO: 333). The PCR was performed using 10 nM of each of the specific primers, 0.667 μM of the biotinylated Universal Primer A, 1 μM of the biotinylated Universal Primer B, 1× QIAGEN Multiplex PCR Master Mix (Qiagen, Hilden, Germany), 600 μM dUTP (Roche, Manheim, Germany), 0.5 U of UNG (USB corp., Cleveland, USA). Samples were first incubated at 22° C. for 10 min (activation of UNG) and then incubated at 94° C. for 15 min (inactivation of UNG/activation of Taq polymerase). The first ten amplification cycles were performed with 94° C. for 30s, 55° C. for 90s and 72° C. for 30° C. The next 30 cycles were performed with 94° C. for 30s, 60° C. for 90s and 72° C. for 30° C. and a final extension step of 10 min at 72° C.

The resulting target amplicons were between 119 and 150 bp long.

The capture nucleotide sequences contained specific binding sequence for their respective target. The specific parts of the capture molecule are presented in the table below.

| | |
|---|---|
| Target GMO | Probe sequence |

| | |
|---|---|
| T25 | TCATTGAGTCGTTCCGCCATTGTCG |
| | (SEQ ID NO: 334) |
| T45 | GAGGACCTAACAGAACTCGCCGT |
| | (SEQ ID NO: 335) |
| MON531 | TTGTCCCTCCACTTCTTCTCTGCTA |
| | (SEQ ID NO: 336) |
| LLRICE62 | GCACCGATTATTTATACTTTTAGTCCAC |
| | (SEQ ID NO: 337) |
| Invertase | TTAGACGGGAAAACGAGAGGAAGC |
| | (SEQ ID NO: 338) |

Each capture probe comprised a spacer at its 5′ end which has the following sequence: ataaaaaagtgggtcttagaaataaatttcgaagtgcaataattattattcacaacatttcgatttttg caactacttcagtt cactccaaatta (SEQ ID NO: 339).

The last nucleotide contained a free amino group for binding on the activated glass.

The capture molecules were chemically synthesised by Eurogentec (Liege, Belgium).

The capture molecules were spotted on Diaglass which are glass slides activated according to the process described in the EP01313677B1.

Each spot of the array was obtained according to the technology developed for the DualChips (Eppendorf; Array Technologies, Namur, Belgium) by deposit at a location on the slide of around 0.2 nl of spotting solution containing the capture molecules at 3 mM.

After amplification, the amplicons were hybridized on the arrays. The hybridization mix containing 9 μl of PCR product, 5 μl of Sensihyb solution (Eppendorf, Hamburg, Germany), 4 μl of hybridization control (Eppendorf, Hamburg, Germany) and 27 μl of water are denaturated with 5 μl of NaOH and then incubated 5 min at room temperature. 50 μl of hybridisation solution (Eppendorf, Hamburg, Germany) were added in the mix and the solution was loaded on the array framed by a hybridisation chamber. The chamber was closed with a covership. The hybridisation was carried out at 60° C. for 1 h. Samples were washed with several washing buffers as described in the DualChip Manual.

The detection was performed in colorimetry using the Siverquant labeling provided by Eppendorf (Hamburg, Germany) and described in EP1179180B1. In short, the glass samples were first incubated 45 min at room temperature with colloidal gold-conjugated IgG Anti-biotin 1000× diluted in blocking buffer. After 5 washes with washing buffer, the presence of gold served for catalysis of silver reduction using a staining revelation solution. The slides were then incubated 3 times 10 min with the revelation mixture, then rinsed with water, dried and analysed using the Silverquant scanner. Each slide was then quantified by the Silverquant data analysis software. Data were corrected for the local background and the triplicates were averaged.

Results

Signals for the four GMO and for the maize reference gene (invertase) were detected. Signal intensity after hybridization of PCR products are given in the table below as the raw values after background subtraction on a scale or 65 536.

| | Signal intensity | |
|---|---|---|
| Capture probe | With universal primers | Without universal primers |
| T45 | ND | ND |
| MON531 | 55178 | 129 |
| LLRICE62 | 57591 | 0 |
| T25 | 34456 | 0 |
| Invertase | 44558 | 9064 |

The table also gives the signals values when the universal primers are not incorporated into the PCR. ND is not detectable value considered as absent. The experiments were repeated two times and lead to identical conclusion concerning the presence of the four different GMO and of the maize plant reference gene (invertase).

### EXAMPLE 25

### Amplification and Detection of 13 Different GMO (Genetically Modified Organisms)

The list of the targeted GMO to be detected is presented in the table below together with the two primers used for each of the gene sequence to be amplified. Two common sequences are present on the two primers of the same pair. These common sequences which are later used as universal amplifying sequences are in bold. Specific sequences are in italics.

| Target | | |
|---|---|---|
| GMO | Primer forward | Primer reverse |
| T25 | TCTATATGCTCCACAGTATGC | ACTATATGCTCCACTCTATGC |
| | GAACAAGCGTGTCGTGCTCCA | CTCGACATGATACTCCTTCCA |
| | (SEQ ID NO: 340) | C |
| | | (SEQ ID NO: 341) |
| T45 | TCTATATGCTCCACAGTATGC | ACTATATGCTCCACTCTATGC |
| | GACAATGGACACATGAATTAT | CTGACTCTGTATGAACTGTTC |
| | G | G |
| | (SEQ ID NO: 342) | (SEQ ID NO: 343) |
| MON531 | TCTATATGCTCCACAGTATGC | ACTATATGCTCCACTCTATGC |
| | GATTTCCCATTCGAGTTTCTC | CTAACCAATGCCACCCCACTG |
| | AC | (SEQ ID NO: 345) |
| | (SEQ ID NO: 344) | |
| LLRICE62 | TCTATATGCTCCACAGTATGC | ACTATATGCTCCACTCTATGC |
| | GACTGGCGTAATAGCGAAGAG | CTCTAACGGGTGCATCGTCTA |
| | (SEQ ID NO: 346) | (SEQ ID NO: 347) |
| GT73 | TCTATATGCTCCACAGTATGC | ACTATATGCTCCACTCTATGC |
| | GACATATTGACCATCATACTT | CTTTATACGAAGGCAAGAAAA |
| | GCT | GCA |
| | (SEQ ID NO: 348) | (SEQ ID NO: 349) |
| NK603 | TCTATATGCTCCACAGTATGC | ACTATATGCTCCACTCTATGC |
| | GAATGAATGACCTCGAGTAAG | CTAAGAGATAACAGGATCCAC |
| | CTTGTTAA | TCA |
| | (SEQ ID NO: 350) | (SEQ ID NO: 351) |
| Bt11 | TCTATATGCTCCACAGTATGC | ACTATATGCTCCACTCTATGC |
| | GAGCGGAACCCCTATTTGT | CTTCCAAGAATCCCTCCATG |
| | (SEQ ID NO: 352) | (SEQ ID NO: 353) |
| RRS | TCTATATGCTCCACAGTATGC | ACTATATGCTCCACTCTATGC |
| | GACCTTTAGGATTTCAGCATC | CTGACTTGTCGCCGGGA |
| | A | (SEQ ID NO: 355) |
| | (SEQ ID NO: 354) | |
| EH92- | TCTATATGCTCCACAGTATGC | ACTATATGCTCCACTCTATGC |
| 527-1 | GATGTCAAAACACAATTTACA | CTTCCCTTAATTCTCCGCTC |
| | GC | (SEQ ID NO: 357) |
| | (SEQ ID NO: 356) | |
| H7-1 | TCTATATGCTCCACAGTATGC | ACTATATGCTCCACTCTATGC |
| | GAGGATCTGGGTGGCTCTAA | CTAATGCTGCTAAATCCTGAG |
| | (SEQ ID NO: 358) | (SEQ ID NO: 359) |
| MON863 | TCTATATGCTCCACAGTATGC | ACTATATGCTCCACTCTATGC |
| | GATAGGATCGGAAAGCTTGG | CTTTACGGCCTAAATGCTGA |
| | (SEQ ID NO: 360) | (SEQ ID NO: 361) |
| 1507 | TCTATATGCTCCACAGTATGC | ACTATATGCTCCACTCTATGC |
| | GATAGTCTTCGGCCAGAATG | CTCTTTGCCAAGATCAAGCG |
| | (SEQ ID NO: 362) | (SEQ ID NO: 363) |
| 59122 | TCTATATGCTCCACAGTATGC | ACTATATGCTCCACTCTATGC |
| | GAGGATAAGCAAGTAAAAGCG | CTCTTAATTCTCCGCTCATGA |
| | (SEQ ID NO: 364) | T |
| | | (SEQ ID NO: 365) |

The PCR was performed on 100 ng DNA extracted from the samples using the standard CTAB procedure of Rogers and Bendich (1985, Plant Biol. 5:69-76). The PCR solution contained both the specific and the universal primers. The universal primers were TCTATATGCTCCACAGTATGCGA (SEQ ID NO: 366) and ACTATATGCTCCACTCTATGCCT (SEQ ID NO: 367). The PCR was performed using 10 nM of each of the specific primers, 1 μM of each biotinylated universal primer, 1× QIAGEN Multiplex PCR Master Mix (Qiagen, Hilden, Germany), 600 μM dUTP (Roche, Manheim, Germany), 0.5 U of UNG (USB corp., Cleveland, USA), 100 ng of target genomic DNA in a final volume of 25 μl. Samples were first incubated at 22° C. for 10 min (activation of UNG) and then incubated at 94° C. for 15 min (inactivation of UNG/activation of Taq polymerase). The first ten amplification cycles were performed with 94° C. for 30s, 55° C. for 90s and 72° C. for 30° C. The next 30 cycles were performed with 94° C. for 30s, 60° C. for 90s and 72° C. for 30° C. and a final extension step of 10 min at 72° C.

The resulting target amplicons were between 104 and 180 bp long.

The capture nucleotide sequences contained specific binding sequence for their respective target. The specific parts of the capture molecule are presented in the table below.

| | |
|---|---|
| Target GMO | Probe sequence |

| | |
|---|---|
| T25 | TCATTGAGTCGTTCCGCCATTGTCG |
| | (SEQ ID NO: 368) |
| T45 | GAGGACCTAACAGAACTCGCCGT |
| | (SEQ ID NO: 369) |
| MON531 | TTGTCCCTCCACTTCTTCTCTGCTA |
| | (SEQ ID NO: 370) |
| LLRICE62 | GCACCGATTATTTATACTTTTAGTCCAC |
| | (SEQ ID NO: 371) |
| GT73 | TTCCCGGACATGAAGATCATGGTCCTT |
| | (SEQ ID NO: 372) |
| NK603 | GTACCACGCGACACACTTCCACT |
| | (SEQ ID NO: 373) |
| Bt11 | AAATACATTCAAATATGTATCCGCTCA |
| | (SEQ ID NO: 374) |
| RRS | CGCAACCGCCCGCAAATCC |
| | (SEQ ID NO: 375) |
| EH92-527-1 | AGATTGTCGTTTCCCGCCTTCAGTT |
| | (SEQ ID NO: 376) |
| H7-1 | AAGGCGGGAAACGACAATCT |
| | (SEQ ID NO: 377) |
| MON863 | TGAACACCCATCCGAACAAGTAGG |
| | (SEQ ID NO: 378) |
| 1507 | TAACTCAAGGCCCTCACTCCG |
| | (SEQ ID NO: 379) |
| 59122 | TTTAAACTGAAGGCGGGAAACGACAA |
| | (SEQ ID NO: 380) |

Each capture probe comprised a spacer at its 5′ end which has the following sequence: ataaaaaagtgggtcttagaaataaatttcgaagtgcaataattattattcacaacatttcgatttttgcaa ctacttcagtcactccaaatta (SEQ ID NO: 381).

The last nucleotide contains a free amino group for binding on the activated glass.

The capture molecules were chemically synthesised by Eurogentec (Liege Belgium).

The capture molecules were spotted on Diaglass which are glass slides activated according to the process described in the EP01313677B1.

Each spot of the array was obtained according to the technology developed for the DualChips (Eppendorf; Array Technologies, Namur, Belgium) by deposit at a location on the slide of around 0.2 nl of spotting solution containing the capture molecules at 3 mM.

The hybridization, detection n the arrays and the quantification were performed as in Example 24.

### EXAMPLE 26

### Amplification and Detection of SNP in bacterial Genome

The experiment was conducted as described in Example 24.

The list of the targeted genes is presented in the Table below together with the two primers used for each of the gene sequence to be amplified. The two universal amplifying sequences are in bold. Specific sequences are in italics. The SNP are present in Pseudomonas aeruginosa.

| | | |
|---|---|---|
| Gene | Sense primer (5′ -> 3′) | Antisense primer (5′ -> 3′) |

| | | |
|---|---|---|
| gyrA | TCTATATGCTCCACAGTATGCG | ACTATATGCTCCACTCTATGC |
| | AGACGGCCTGAAGCCGGTGCAC | CTGCCCACGGCGATACCGCTG |
| | (SEQ ID NO: 382) | GA |
| | | (SEQ ID NO: 383) |
| gyrB | TCTATATGCTCCACAGTATGCG | ACTATATGCTCCACTCTATGC |
| | ACCTGACCATCCGTCGCCACAA | CTCGCAGCAGGATGAAGACGC |
| | C | C |
| | (SEQ ID NO: 384) | (SEQ ID NO: 385) |
| oprL | TCTATATGCTCCACAGTATGCG | ACTATATGCTCCACTCTATGC |
| | AGCTCTGGCTCTGGCTGCT | CTAGGGCACGCTCGTTAGCC |
| | (SEQ ID NO: 386) | (SEQ ID NO: 387) |
| mexR | TCTATATGCTCCACAGTATGCG | ACTATATGCTCCACTCTATGC |
| | AGATGCCCGCGCTGATGG | CTAGGCACTGGTCGAGGAGAT |
| | (SEQ ID NO: 388) | G |
| | | (SEQ ID NO: 389) |

The method was based on a two-step PCR. The PCR reaction was performed using 0.05 μM of each of the universal primers, 1 μM of each two biotinylated universal primers 1× TAQ polymerase reaction buffer (Eppendorf, Hamburg, Germany), 200 ∥M dNTP, 2.5 U of Taq polymerase (Eppendorf, Hamburg, Germany), 100 ng of target genomic DNA in a final volume of 25 μl. Samples were first denatured at 95 C for 2 min. The first ten amplification cycles were performed with 95° C. for 30s, 55° C. for 30s and 72° C. for 30° C. The next 30 cycles were performed with 95° C. for 30s, 60° C. for 30s and 72° C. for 30° C. and a final extension step of 10 min at 72° C.

The resulting target amplicons were between 197 and 417 bp long.

The capture nucleotide sequences contained specific binding sequence for their respective SNP target. Wt represents the wild type sequence while the mut represents the mutated gene. The specific parts of the capture molecules are presented in the Table below.

| | |
|---|---|
| Target SNP | Probe sequence |

| | |
|---|---|
| 248 gyrA wt | TAGCCGCGGTGTCGCCTG |
| | (SEQ ID NO: 390) |
| 248 gyrA mut | TAGCCGCGCTGTCGCCTG |
| | (SEQ ID NO: 391) |
| 260 gyrA wt | CACGATGGTGTCGTAGACCGC |
| | (SEQ ID NO: 392) |
| 260 gyrA mut | CACGATGGTGCCGTAGACCGC |
| | (SEQ ID NO: 393) |
| gyrB as | CTGAAGTGGATGTTGCTGAAGGTC |
| | (SEQ ID NO: 394) |
| oprI as | CGTCTTCGGTAGCGGTCAG |
| | (SEQ ID NO: 395) |
| 165 mexR wt | TCCCAGGTCCCGCAGGTTCAG |
| | (SEQ ID NO: 396) |
| 165 mexR mut | TCCCAGGTCCCCCAGGTTCAG |
| | (SEQ ID NO: 397) |
| 208 mexR wt | GGATCTTCCGGGTGATCAGT |
| | (SEQ ID NO: 398) |
| 208 mexR mut | GGATCTTCCAGGTGATCAGT |
| | (SEQ ID NO: 399) |
| 264 mexR wt | CGCTGGTCCCTGGGGTTG |
| | (SEQ ID NO: 400) |
| 264 mexR mut | CGCTGGTCCGTGGGGTTG |
| | (SEQ ID NO: 401) |

Each capture probe comprised a spacer at its 5′ end which has the following sequence: ataaaaaagtgggtcttagaaataaatttcgaagtgcaataattattattcacaacatttcgatttttgcaac tacttcagttcactccaaatta (SEQ ID NO: 402).

The last nucleotide contains a free amino group for binding on the activated glass.

After amplification, the amplicons were hybridized on the arrays. The hybridization mix containing 9 μl of PCR product, 5 μl of sensihyb solution (Eppendorf, Hamburg, Germany), 4 μl of hybridization control (Eppendorf, Hamburg, Germany) and 27 μl of water were denaturated with 5 μl of NaOH and then incubated 5 min at room temperature. 50 μl of hybridisation solution (Eppendorf, Hamburg, Germany) was added in the mix and the solution was loaded on the array framed by a hybridisation chamber. The chamber was closed with a covership. The hybridisation was carried out at 60° C. for 1 h. Samples were washed with several washing buffers as described in the DualChip Manual.

The detection was performed in colorimetry using the Siverquant labeling provided by Eppendorf (Hamburg, Germany) and described in EP1179180B1. In short, the glass samples were first incubated 45 min at room temperature with colloidal gold-conjugated IgG Anti-biotin 1000× diluted in blocking buffer. After 5 washes with washing buffer, the presence of gold served for catalysis of silver reduction using a staining revelation solution. The slides were then incubated 3 times 10 min with the revelation mixture, then rinsed with water, dried and analysed using the Silverquant scanner. Each slide was then quantified by the Silverquant data analysis software. Data were corrected for the local background and then averaged.

### EXAMPLE 27

### Amplification and Detection of Different Bacteria Species

The experiment was conducted as described in Example 24.

The list of the targeted bacteria is presented in the Table below together with the two primers used for each of the gene sequence to be amplified. Universal amplifying sequences are in bold. Specific sequences are in italics.

| | Target | |
|---|---|---|
| Target species | genes | Sense primer (5′ -> 3′) |
| Salmnollela spp | invA | GCTATGCTCACAGATGCGACGTCTCTG |
| | | GATGGTATGCCCG |
| | | (SEQ ID NO: 403) |
| L. monocytogenes | hlyA | GCTATGCTCACAGATGCGACGTCCGCC |
| | | TGCAAGTCCTAAG |
| | | (SEQ ID NO: 404) |
| E. coli O157:H7 | eaeA | GCTATGCTCACAGATGCGACGTACACT |
| | | ATAAAAGCACCGTCG |
| | | (SEQ ID NO: 405) |
| E. coli O157:H7 | flicH7 | GCTATGCTCACAGATGCGACGTACTAT |
| | | TACCAACAAAGCTGC |
| | | (SEQ ID NO: 406) |
| C. jejuni | hipO | GCTATGCTCACAGATGCGACGAGGTGC |
| | | GATGATGGCTTCT |
| | | (SEQ ID NO: 407) |
| Y. | yst | GCTATGCTCACAGATGCGACGGTCTTC |
| enterocolitica | | ATTTGGAGCATTCGG |
| | | (SEQ ID NO: 408) |
| V. cholerae | ctxA | GCTATGCTCACAGATGCGACGACAGAG |
| | | TGAGTACTTTGACC |
| | | (SEQ ID NO: 409) |
| V. cholerae | hlyA | GCTATGCTCACAGATGCGACGGACTCC |
| | | TCGGTCAATATCC |
| | | (SEQ ID NO: 410) |
| V. | tdh/ | GCTATGCTCACAGATGCGACGTGACTT |
| parahaemolyticus | trh | CTGGACAAACCG |
| | | (SEQ ID NO: 411) |
| V. vulnificus | vvhA | GCTATGCTCACAGATGCGACGAACTTC |
| | | AAACCGAACTATGAC |
| | | (SEQ ID NO: 412) |
| V. vulnificus | viuB | GCTATGCTCACAGATGCGACGGGTTGG |
| | | GCACTAAAGGCAG |
| | | (SEQ ID NO: 413) |
| C. perfringens | cpe | GCTATGCTCACAGATGCGACGGGAGAT |
| | | GGTTGGATATTAGG |
| | | (SEQ ID NO: 414) |
| | Target | |
| Target species | genes | Antisense primer (5′ -> 3′) |
| Salmnollela spp | invA | GCTATGCTCACAGATGCGACGATAAAC |
| | | TTCATCGCACCGTC |
| | | (SEQ ID NO: 415) |
| L. monocytogenes | hlyA | GCTATGCTCACAGATGCGACGGGCGGC |
| | | ACATTTGTCACTG |
| | | (SEQ ID NO: 416) |
| E. coli O157:H7 | eaeA | GCTATGCTCACAGATGCGACGAACGCT |
| | | GCTCACTAGATGTC |
| | | (SEQ ID NO: 417) |
| E. coli O157:H7 | flicH7 | GCTATGCTCACAGATGCGACGTGTGAC |
| | | TTTATCGCCATTCC |
| | | (SEQ ID NO: 418) |
| C. jejuni | hipO | GCTATGCTCACAGATGCGACGTGTCCT |
| | | GCATTAAAAGCTCC |
| | | (SEQ ID NO: 419) |
| Y. | yst | GCTATGCTCACAGATGCGACGAACATA |
| enterocolitica | | CATCGCAGCAATCC |
| | | (SEQ ID NO: 420) |
| V. cholerae | ctxA | GCTATGCTCACAGATGCGACGATACCA |
| | | TCCATATATTTGGGA |
| | | (SEQ ID NO: 421) |
| V. cholerae | hlyA | GCTATGCTCACAGATGCGACGCCGAGC |
| | | TGGTCATAGATGAA |
| | | (SEQ ID NO: 422) |
| V. | tdh/ | GCTATGCTCACAGATGCGACGATTCTG |
| parahaemolyticus | trh | GAGTTTCATCTAAAT |
| | | (SEQ ID NO: 423) |
| V. vulnificus | vvhA | GCTATGCTCACAGATGCGACGTTCCAA |
| | | CTGCCGTGACAGC |
| | | (SEQ ID NO: 424) |
| V. vulnificus | viuB | GCTATGCTCACAGATGCGACGGCGATA |
| | | AAAGCAGACAGCG |
| | | (SEQ ID NO: 425) |
| C. perfringens | cpe | GCTATGCTCACAGATGCGACGGGACCA |
| | | GCAGTTGTAGATAC |
| | | (SEQ ID NO: 426) |

The PCR reaction was performed using 0.05 μM of each of the specific primers, 1 μM of biotinylated universal primer, 1× TAQ polymerase reaction buffer (Eppendorf, Hamburg, Germany), 200 μM dNTP, 2.5 U of Taq polymerase (Eppendorf, Hamburg, Germany), 100 ng of target genomic DNA in a final volume of 25 μl. Samples were first denatured at 95° C. for 2 min. The first ten amplification cycles were performed with 95° C. for 30s, 55° C. for 30s and 72° C. for 30° C. The next 30 cycles were performed 95° C. for 30s, 60° C. for 30s and 72° C. for 30° C. and a final extension step of 10 min at 72° C.

The resulting target amplicons were between 140 and 346 bp long.

The capture nucleotide sequences contained specific binding sequence for their respective target. The specific parts of the capture molecule are presented in the Table below.

| Target | |
|---|---|
| gene | Probe sequence |
| invA | GCCGGTATTATTGATGCGGATGC |
| | (SEQ ID NO: 427) |
| hlyA | CTTATCGATTTCATCCGCGTGTTTC |
| | (SEQ ID NO: 428) |
| eaeA | CGGTATTGTCAGATATTTATGACTCA |
| | (SEQ ID NO: 429) |
| flicH7 | CTTGTTAACTACCGATGCTGCATTCG |
| | (SEQ ID NO: 430) |
| hipO | TCTGGAGCACTTCCATGACCACC |
| | (SEQ ID NO: 431) |
| yst | GCTTGTGATCCTCCGCTGCCACC |
| | (SEQ ID NO: 432) |
| ctxA | GCAAGAGGAACTCAGACGGGATTTG |
| | (SEQ ID NO: 433) |
| hlyA | CTTATCGATTTCATCCGCGTGTTTC |
| | (SEQ ID NO: 434) |
| tdh/trh | TCTATTTTCACGACTTCAGGCTCAAAA |
| | (SEQ ID NO: 435) |
| vvhA | GAGATGGGCGTGAAACTCAACTATC |
| | (SEQ ID NO: 436) |
| viuB | AATCCTCATGATGCCGAGCCCGCA |
| | (SEQ ID NO: 437) |
| cpe | ATGGATTTGGAATAACTATAGGAGAACA |
| | (SEQ ID NO: 438) |

Each capture probe comprised a spacer at its 5′ end which has the following sequence: ataaaaaagtgggtcttagaaataaatttcgaagtgcaataattattattcacaacatttcgatttttg caactacttcagttcactccaaatta (SEQ ID NO: 439).

The last nucleotide contains a free amino group for binding on the activated glass.

After amplification, the amplicons were hybridized as described in example 28 and the detection is performed in colorimetry using the Silverquant labelling.

### EXAMPLE 28

### Amplification and Quantification of RNA Extracted from Paraffin Embedded Tissue Tissue by PCR Amplification and Detection on Arrays

6 genes were selected as being expressed in the sample or being used as house keeping genes. For each of them a specific primer pair was designed having a specific sequence complementary either of the sense and the other one of the antisense strand. The primer sequence of the 6 different genes was described in the table below. Each of the different primers had an additional 5′ end universal sequence being; TGCTATGCTCACAGATGCGA (SEQ ID NO: 440). The lengths of amplified targets were comprised between 80 bp and 107 bp. The same sequence TGCTATGCTCACAGATGCGA (SEQ ID NO: 441) was used for the universal primer.

Primer sequences

| | Sense primer | Antisense primer |
|---|---|---|
| Genes | (5′ -> 3′) | (5′ -> 3′) |
| CCNE1 | TGACCTAAGGGACTCCCAC | GTACAACGGAGCCCAGAA |
| | AA | CAC |
| | (SEQ ID NO: 442) | (SEQ ID NO: 443) |
| GATA3 | CAAAGGAGCTCACTGTGGT | GGGATATGAGTCAGAATG |
| | GTCT | GCTTATTC |
| | (SEQ ID NO: 444) | (SEQ ID NO: 445) |
| MCM7 | TGGATGAATATGAGGAGCT | AGCAGGCTGGAATCAGAC |
| | CAATG | AAA |
| | (SEQ ID NO: 446) | (SEQ ID NO: 447) |
| TFF1 | CCCTCCCAGTGTGCAAATA | GGACGTCGATGGTATTAG |
| | AG | GATAGAA |
| | (SEQ ID NO: 448) | (SEQ ID NO: 449) |
| K-ALPHA-1 | AATACATGGCTTGCTGCCT | CGTGCGCTTGGTTTTGAT |
| | GTT | G |
| | (SEQ ID NO: 450) | (SEQ ID NO: 451) |
| YWHAZ | TTGACATTGTGGACATCGG | AAGTTGGAAGGCCGGTTA |
| | ATAC | ATTT |
| | (SEQ ID NO: 452) | (SEQ ID NO: 453) |

Total RNA Extraction

The total RNA extraction was performed using the RecoverAll Total Nucleic Acid Isolation kit from Ambion (Cat#1975). Part I of the isolation, the “Deparaffinization”, was performed starting from Fresh Frozen Paraffin Embedded Human Tonsil Thin section thinner than 80 μm, in a final volume of 1 ml 100% Xylene (Fisher, #0287K) and by incubating for 3 min at 50° C. then centrifuging for 2 min at maximum speed. The digested pellet was washed twice with 1 ml 100% ethanol (MERCK, 8.18760.1000). Part II, the “Protease digestion”, was performed by adding Digestion buffer and Protease then by incubating for 16 h at 50° C. Part III, the “Nucleic Acid Isolation, was performed by adding 480 μl Isolation additive and 1.1 ml 100% ethanol (MERCK, 8.18760.1000) then filtering and washing. Part IV, the “Nuclease Digestion and Final Purification” was performed by incubating 60 μl of DNase for 30 min at room temperature, then by washing and eluting twice in 30 μl nuclease free water.

FFPE extracted RNA yield was assessed in a ND-1000 spectrophotometer (Nanodrop Technologies, Inc. Wilmington, USA) and size distribution was obtained by capillary electrophoresis with the Agilent 2100 BioAnalyzer® (Agilent Technologies, Palo Alto, Calif.).

RT-PCR

RT-PCR was performed using an amplification kit from Promega (Access RT-PCR system, Cat# A1250). The RT-PCR was performed in a final volume of 50 μl the following reagents were added in a reaction tube: 1× AMV/Tfl 5× Reaction Buffer, 200 μM of dNTP mix, 50 nM of each specific primer, 1 μM of the universal primer, 1 mM of MgSO₄, 5U of AMV Reverse Transcriptase (5 U/μl), 5 U of Tfl DNA Polymerase (5 U/μl), 1 μg of Breast Adenocarcinoma (MCF7) Total RNA from Ambion (Cat# AM7846), 32 μM of biotin-11-dATP (Perkin Elmer, NEL540, 1 mM) and 32 μM of biotin-11-dCTP (Perkin Elmer, NEL538, 1 mM).

The reaction tubes were then placed in a thermocycler programmed as follows: (i) reverse transcription of 45 min at 48° C., (ii) AMV RT inactivation at 94° C. for 2 min, (iii) 35 PCR cycles including a denaturation step of 30 sec at 94° C., annealing step of 60 sec at 54° C. and extension step of 2 min at 68° C. and a final extension step of 7 min at 68° C.

Water controls were used as negative controls of the amplification.

Microarray

DualChip human breast (Eppendorf, Hamburg, Germany) were used for the detection and the quantification of the amplified sequences. The DualChips were obtained by spotting aminated capture molecules on aldehyde activated glass obtained according to the EP01313677B1, the disclosure of which is incorporated herein by reference in its entirety, using a home made robotic device. The capture molecules were part of an Xmer technology of Eppendorf and were between 200 and 450 bp long. The spots were around 250 μm in diameter. The slides were stored at 4° C.

The capture probes for the different genes detected in this example are presented in the table below. Their sequences are complementary of the gene transcripts which have to be detected. The sequence complementary of the amplified target sequence is shown in bold. The sequence located in the 5′ end of the capture molecules serves as spacer for the binding of the target amplified sequences.

Capture probe sequence for the different detected genes. The sequence complementary of the amplified target sequence is shown in bold.

| | |
|---|---|
| Genes | Capture probe (5′-3′) |

| | |
|---|---|
| CCNE1 | CCTTCTCCACCAAAGACAGTTGCGCGCCTGCTCCACGTTCT |
| | CTTCTGTCTGTTGCAGCGGAGGCGTGCGTTTGCTTTTACAG |
| | ATATCTGAATGGAAGAGTGTTTCTTCCACAACAGAAGTATT |
| | TCTGTGGATGGCATCAAACAGGGCAAAGTGTTTTTTATTGA |
| | ATGCTTATAGGTTTTTTTTAAATAAGTGGGTCAAGTACACC |
| | AGCCACCTCCAGACACCAGTGCGTGCTCCCGATGCTGCTAT |
| | GGAAGGTGCTACTTGACCTAAGGGACTCCCACAACAACAAA |
| | AGCTTGAAGCTGTGGAGGGCCACGGTGGCGTGGCTCTCCTC |
| | GCAGGTGTTCTGGGCTCCGTTGTACCAAGTGGAGCAGGTGG |
| | TTGCGGGCAAGCGTTGTGCAGAGCCCATAGCCA |
| | (SEQ ID NO: 454) |
| GATA3 | GCCATCCAGCCTGTCCTTTGGACCACACCACCCCTCCAGCA |
| | TGGTCACCGCCATGGGTTAGAGCCCTGCTCGATGCTCACAG |
| | GGCCCCCAGCGAGAGTCCCTGCAGTCCCTTTCGACTTGCAT |
| | TTTTGCAGGAGCAGTATCATGAAGCCTAAACGCGATGGATA |
| | TATGTTTTTGAAGGCAGAAAGCAAAATTATGTTTGCCACTT |
| | TGCAAAGGAGCTCACTGTGGTGTCTGTGTTCCAACCACTGA |
| | ATCTGGACCCCATCTGTGAATAAGCCATTCTGACTCATATC |
| | CCCTATTTAACAGGGTCTCTAGTGCTGTGAAAAAAAAAAAT |
| | CCTGAACATTGCATATAACTTATATTGTAAGAAATACTGTA |
| | CAATGACTTTATTGCATCTGGGTAGCTGTAAGGCATGAAGG |
| | ATGCCAAGAAGTTT |
| | (SEQ ID NO: 455) |
| MCM7 | TGAGAATGGTGGATGTGGTGGAGAAAGAAGATGTGAATGAA |
| | GCCATCAGGCTAATGGAGATGTCAAAGGACTCTCTTCTAGG |
| | AGACAAGGGGCAGACAGCTAGGACTCAGAGACCAGCAGATG |
| | TGATATTTGCCACCGTCCGTGAACTGGTCTCAGGGGGCCGA |
| | AGTGTCCGGTTCTCTGAGGCAGAGCAGCGCTGTGTATCTCG |
| | TGGCTTCACACCCGCCCAGTTCCAGGCGGCTCTGGATGAAT |
| | ATGAGGAGCTCAATGTCTGGCAGGTCAATGCTTCCCGGACA |
| | CGGATCACTTTTGTCTGATTCCAGCCTGCTTGCAACCCTGG |
| | GGTCCTCTTGTTCCCTGCTGGCCTGCCCCTTGGGAAGGGGC |
| | AGTGATGCCTTTGAGGGGAAGGAGGAGCCCCTCTTTCTCCC |
| | ATGCTGCACT |
| | (SEQ ID NO: 456) |
| TFF1 | GGAGCAGAGAGGAGGCAATGGCCACCATGGAGAACAAGGTG |
| | ATCTGCGCCCTGGTCCTGGTGTCCATGCTGGCCCTCGGCAC |
| | CCTGGCCGAGGCCCAGACAGAGACGTGTACAGTGGCCCCCC |
| | GTGAAAGACAGAATTGTGGTTTTCCTGGTGTCACGCCCTCC |
| | CAGTGTGCAAATAAGGGCTGCTGTTTCGACGACACCGTTCG |
| | TGGGGTCCCCTGGTGCTTCTATCCTAATACCATCGACGTCC |
| | CTCCAGAAGAGGAGTGTGAATTTTAGACACTTCTGCAGGGA |
| | TCTGCCTGCATCCTGACGGGGTGCCGTCGCCAGCACGGTGA |
| | TTAGTCCCAGAGCTCGGCTGCCACCTCCACCGGACACCTCA |
| | GACACGCTTCTGCAGCTGTGCCTCGGCTCACAACACAGATT |
| | GACTGCTCTGACTTTGAC |
| | (SEQ ID NO: 457) |
| K-ALPHA-1 | GCCAACCAGATGGTGAAATGTGACCGTGGCCATGGTAAATA |
| | CATGGCTTGCTGCCTGTTGTACCGTGGTGACGTGGTTCCCA |
| | AAGATGTCAATGCTGCCATTGCCACCATCAAAACCAAGCGC |
| | ACGATCCAGTTTGTGGATTGGTGCCCCACTGGCTTCAAGGT |
| | TGGCATCAACTACCAGCCTCCCACTGTGGTGCCTGGTGGAG |
| | ACCTGGCCAAGGTACAGAGAGCTGTGTGCATGCTGAGCAAC |
| | ACCACAGCCATTGCTGAGGCCTGGGCTCGCCTGGACCACAA |
| | GTTTGACCTGATGTATGCCAAGCGTGCCTTTGTTCACTGGT |
| | ACGTGGGTGAGGGGATGGAGGAAGGCGAGTTTTCAGAGGCC |
| | CGTGAAGATATGGCTGCCCTTGAGAAGGATTATGAGGAGGT |
| | TGGTGTGGATTCTGTTGAAGGAGAGGGTGAGGAAGAAGGAG |
| | AGGAATACTA |
| | (SEQ ID NO: 458) |
| YWHAZ | GACAGCACGCTAATAATGCAATTACTGAGAGACAACTTGAC |
| | ATTGTGGACATCGGATACCCAAGGAGACGAAGCTGAAGCAG |
| | GAGAAGGAGGGGAAAATTAACCGGCATACCCAAGGAGACGA |
| | AGCTGAAGCAGGAGAAGGAGGGGAAAATTAACCGGCCTTCC |
| | AACTTTTGTCTGCCTCATTCTAAAATTTACACAGTAGACCA |
| | TTGTCATCGATGCTGTGCCACAAATAGTTTTTTGTTTACGA |
| | TTTATGACAGGTTTATGTTACTTCTATTTGAATTTCTATAT |
| | TTCCCATGTGGTTTTTATGTTTAATATTAGGGGAGTAGAGC |
| | CAGTTAACATTTAGGGAGTTATCTGTTTTCATCTTGAGGTG |
| | GCCAATATGGGGATGTGGAATTTTTATACAAGTTATAAGTG |
| | TTTGGCATAGTACTTTTGGTACATTGTGGCTTCAAAAGGGC |
| | CAGTGTAAAACTGCTTCCATGTCTAAGCAAAGAAAACTGCC |
| | TACATACTGGATTTGTCCGTGGCGGGGAATAAAAGGGATCA |
| | (SEQ ID NO: 459) |

Hybridization

For each condition, 10 μl of PCR product were added to the hybridization mix (Eppendorf, Hamburg, Germany) to a final volume of 100 μl. The hybridization mix was injected slowly by the injection port of the hybridization frame of the DualChip. The frame was sealed with an aluminium pad and immediately after the sealing, the slides were placed in the Thermomixer comfort (Eppendorf) and incubated overnight (for 12-16 h) at 60° C.

After the hybridization step, the slides were washed 4 times for 2 min with a washing buffer as described in the DualChip Manual.

Fluorescence Detection

The slides were incubated 45 min at room temperature with the Cy3-conjugated IgG Anti-biotin (Jackson Immuno Research Laboratories, Inc #200-162-096) diluted 1/1000× Conjugate-Cy3 in the blocking buffer and protected from light. After this incubation, the slides were washed 5 times for 2 min with the washing buffer and 2 times with distilled water for 2 min and then these slides were dried before being stored at room temperature. The detection was performed in a confocal laser scanner “Autoloader ScanArray” (Packard, USA) and quantified by a specific quantification software. The signal intensity for each spot is corrected by the subtraction of the local background and then averaged. The quantification process was described in detail by de Longueville et al. (2002 Biochem. Pharmacol. 64:137-149).

Results

The extraction was performed on 4 slices of 10 μM FFPE Human tonsil and 1 μg of extracted total RNA was used in RT-PCR amplification.

The signal intensities of hybridization on DualChip human breast cancer for the different amplified gene transcripts are given in the table below. The scale of the scanner is from 1 to 65536.

Signal intensities of 6 amplified gene transcripts after hybridization on DualChip human breast cancer.

| | Replicate 1 | Replicate 2 | Mean | CV |
|---|---|---|---|---|
| CCNE1 | 11277 | 14515 | 12896 | 17.8 |
| GATA3 | 5109 | 4782 | 4946 | 4.7 |
| MCM7 | 14693 | 6915 | 10804 | 50.9 |
| TFF1 | 6305 | 4470 | 5388 | 24.1 |
| RALPHA1 | 21575 | 18833 | 20204 | 9.6 |
| YWHAZ | 7472, | 5265 | 6369 | 24.5 |

### EXAMPLE 29

### Amplification and Quantification of RNA Extracted from Paraffin Embedded Tissue

Total RNA Extraction

The extraction was performed on 4 slices of 10 μM FFPE Human breast cancer samples from two different patients and 100 ng of extracted total RNA was used in RNA amplification. The patients were characterized based on the expression status of the estrogen receptor-α (ERα, gene ESR1).

The total RNA extraction was performed using the RecoverAll Total Nucleic Acid Isolation kit from Ambion (Cat#1975). Part I of the isolation, the “Deparaffinization”, was performed starting from Fresh Frozen Paraffin Embedded Human breast cancer samples Thin section of 10 μm, in a final volume of 1 ml 100% Xylene (Fisher, #0287K) and for 3 min at 50° C., then centrifuging for 2 min at maximum speed. The digested pellet was washed twice with 1 ml 100% ethanol (MERCK, 8.18760.1000). Part II, the “Protease digestion”, was performed by adding Digestion buffer and Protease, then by incubating for 16 h at 50° C. Part III, the “Nucleic Acid Isolation”, was performed by adding 480 μl Isolation additive and 1.1 ml 100% ethanol (MERCK, 8.18760.1000) then filtering and washing. Part IV, the “Nuclease Digestion and Final Purification” was performed by incubating samples with 60 μl of DNase for 30 min at room temperature, then by washing and eluting twice in 30 μl nuclease free water.

FFPE extracted RNA yield was assessed in a ND-1000 spectrophotometer (Nanodrop Technologies, Inc. Wilmington, USA) and size distribution was obtained by capillary electrophoresis with the Agilent 2100 BioAnalyzer® (Agilent Technologies, Palo Alto, Calif.).

Amplification

One hundred ng of total RNA was amplified and biotin labelled using a whole transcriptome amplification kit from Nugen (WT Ovation™ Pico RNA amplification system) according to manufacturer's instructions. The amplified product was a single stranded cDNA in the antisense direction. After the amplification, 5 μg of purified amplified cDNA was labelled using NuGEN's FL-Ovation™ cDNA Biotin Module V2. The biotin labelling was performed in a final volume of 50 μl. The following reagents were added in a reaction tube: 15 μl of labelling Buffer mix, 1.5 μl of Labelling Reagent, 1.5 μl of Labelling Enzyme mix, 64 μM of biotin-11-dATP (Perkin Elmer, NEL540, 1 mM) and 64 μM of biotin-11-dCTP (Perkin Elmer, NEL538, 1 mM) and 5 μg of purified amplified cDNA. After the biotin labelling, the labelled cDNA was purified using the CyScribe GFX purification (Amersham) according to the instruction's manual.

The reaction tubes were then placed in a thermocycler programmed as follows: (i) Labeling of 60 min at 37° C., (ii) 10 min at 70° C. and (iii) forever at 4° C.

Microarray

DualChip human breast (Eppendorf, Hamburg, Germany) were used for the detection and the quantification of the amplified sequences. The DualChips were obtained by spotting aminated capture molecules on aldehyde activated glass obtained according to the EP01313677B1 using a home made robotic device. The capture molecules were part of an Xmer technology of Eppendorf and are between 200 and 450 bp long. The spots were around 250 μm in diameter. The slides were stored at 4° C.

The capture probes for the different genes detected in this example are presented in the table below. Their sequences are complementary of the gene transcripts which have to be detected.

Capture probe sequence for the different detected genes.

| | |
|---|---|
| Genes | Capture sequence (5′-3′) |

| | |
|---|---|
| BCL2 | AGCACAGAAGATGGGAACACTGGTGGAGGATGGAAAGGCTC |
| | GCTCAATCAAGAAAATTCTGAGACTATTAATAAATAAGACT |
| | GTAGTGTAGATACTGAGTAAATCCATGCACCTAAACCTTTT |
| | GGAAAATCTGCCGTGGGCCCTCCAGATAGCTCATTTCATTA |
| | AGTTTTTCCCTCCAAGGTAGAATTTGCAAGAGTGACAGTGG |
| | ATTGCATTTCTTTTGGGGAAGCTTTCTTTTGGTGGTTTTGT |
| | TTATTATACCTTCTTAAGTTTTCAACCAAGGTTTGCTTTTG |
| | TTTTGAGTTACTGGGGTTATTTTTGTTTTAAATAAAAATAA |
| | GTGTACAATAAGTGTTTTTGTATTGAAAGCTTTTGTTATCA |
| | AGATTTTCATACTTTTACCTTCCATGGCTCTTTTTAAGATT |
| | GATACTTTTAAGAGGTGGCTG |
| | (SEQ ID NO: 479) |
| CCNE1 | CCTTCTCCACCAAAGACAGTTGCGCGCCTGCTCCACGTTCT |
| | CTTCTGTCTGTTGCAGCGGAGGCGTGCGTTTGCTTTTACAG |
| | ATATCTGAATGGAAGAGTGTTTCTTCCACAACAGAAGTATT |
| | TCTGTGGATGGCATCAAACAGGGCAAAGTGTTTTTTATTGA |
| | ATGCTTATAGGTTTTTTTTAAATAAGTGGGTCAAGTACACC |
| | AGCCACCTCCAGACACCAGTGCGTGCTCCCGATGCTGCTAT |
| | GGAAGGTGCTACTTGACCTAAGGGACTCCCACAACAACAAA |
| | AGCTTGAAGCTGTGGAGGGCCACGGTGGCGTGGCTCTCCTC |
| | GCAGGTGTTCTGGGCTCCGTTGTACCAAGTGGAGCAGGTGG |
| | TTGCGGGCAAGCGTTGTGCAGAGCCCATAGCCA |
| | (SEQ ID NO: 480) |
| ESR1 | CCATCGTCAGTGTGTGTGTTTAGAGCTGTGCACCCTAGAAA |
| | CAACATACTTGTCCCATGAGCAGGTGCCTGAGACACAGACC |
| | CCTTTGCATTCACAGAGAGGTCATTGGTTATAGAGACTTGA |
| | ATTAATAAGTGACATTATGCCAGTTTCTGTTCTCTCACAGG |
| | TGATAAACAATGCTTTTTGTGCACTACATACTCTTCAGTGT |
| | AGAGCTCTTGTTTTATGGGAAAAGGCTCAAATGCCAAATTG |
| | TGTTTGATGGATTAATATGCCCTTTTGCCGATGCATACTAT |
| | TACTGATGTGACTCGGTTTTGTCGCAGCTTTGCTTTGTTTA |
| | ATGAAACACACTTGTAAACCTCTTTTGCACTTTGAAAAAGA |
| | ATCCAGCGGG |
| | (SEQ ID NO: 481) |
| GATA3 | GCCATCCAGCCTGTCCTTTGGACCACACCACCCCTCCAGCA |
| | TGGTCACCGCCATGGGTTAGAGCCCTGCTCGATGCTCACAG |
| | GGCCCCCAGCGAGAGTCCCTGCAGTCCCTTTCGACTTGCAT |
| | TTTTGCAGGAGCAGTATCATGAAGCCTAAACGCGATGGATA |
| | TATGTTTTTGAAGGCAGAAAGCAAAATTATGTTTGCCACTT |
| | TGCAAAGGAGCTCACTGTGGTGTCTGTGTTCCAACCACTGA |
| | ATCTGGACCCCATCTGTGAATAAGCCATTCTGACTCATATC |
| | CCCTATTTAACAGGGTCTCTAGTGCTGTGAAAAAAAAAAAT |
| | CCTGAACATTGCATATAACTTATATTGTAAGAAATACTGTA |
| | CAATGACTTTATTGCATCTGGGTAGCTGTAAGGCATGAAGG |
| | ATGCCAAGAAGTTT |
| | (SEQ ID NO: 482) |
| MKI67 | GTATGGTAACTTCTCTGAGCTTCAGTTTCCAAGTGAATTTC |
| | CATGTAATAGGACATTCCCATTAAATACAAGCTGTTTTTAC |
| | TTTTTCGCCTCCCAGGGCCTGTGGGATCTGGTCCCCCAGCC |
| | TCTCTTGGGCTTTCTTACACTAACTCTGTACGTACCATCTC |
| | CTGCCTCCCTTAGGCAGGCACCTCCAACCACCACACACTGC |
| | CTGCTGTTTTCCCTGCCTGGAACTTTCCCTCCTGCCCCACC |
| | AAGATCATTTCATCCAGTCCTGAGCTCAGCTTAAGGGAGGC |
| | TTCTTGCCTGTGGGTTCCCTCACCCCCATGCCTGTCCTCCA |
| | GGCTGGGGCAGGTTCTTAGTTTGCCTGGAATTGTTCTGTAC |
| | CTCTTTGTAGCACGTAGTGTTGTGGAAACTAAGCCACTAAT |
| | TGAGTTTCTGGCTCCCCTCCTGGGGTTGTAAGTTTTGTTCA |
| | TTCA |
| | (SEQ ID NO: 483) |
| SLC39A6 | GCTGTTCTACTAAAGGCTGGCATGACCGTTAAGCAGGCTGT |
| | CCTTTATAATGCATTGTCAGCCATGCTGGCGTATCTTGGAA |
| | TGGCAACAGGAATTTTCATTGGTCATTATGCTGAAAATGTT |
| | TCTATGTGGATATTTGCACTTACTGCTGGCTTATTCATGTA |
| | TGTTGCTCTGGTTGATATGGTACCTGAAATGCTGCACAATG |
| | ATGCTAGTGACCATGGATGTAGCCGCTGGGGGTATTTCTTT |
| | TTACAGAATGCTGGGATGCTTTTGGGTTTTGGAATTATGTT |
| | ACTTATTTCCATATTTGAACATAAAATCGTGTTTCGTATAA |
| | ATTTCTAGTTAAGGTTTAAATGCTAGAGTAGCTTAAAAAGT |
| | TGTCATAGTTTCAGTAGGTCA |
| | (SEQ ID NO: 484) |
| MCM7 | TGAGAATGGTGGATGTGGTGGAGAAAGAAGATGTGAATGAA |
| | GCCATCAGGGTAATGGAGATGTCAAAGGACTCTCTTCTAGG |
| | AGACAAGGGGCAGACAGCTAGGACTCAGAGACCAGCAGATG |
| | TGATATTTGCCACCGTCCGTGAACTGGTCTCAGGGGGCCGA |
| | AGTGTCCGGTTCTCTGAGGCAGAGCAGCGCTGTGTATCTCG |
| | TGGCTTCACACCCGCCCAGTTCCAGGCGGCTCTGGATGAAT |
| | ATGAGGAGCTCAATGTCTGGCAGGTCAATGCTTCCCGGACA |
| | CGGATCACTTTTGTCTGATTCCAGCCTGCTTGCAACCCTGG |
| | GGTCCTCTTGTTCCCTGCTGGCCTGCCCCTTGGGAAGGGGC |
| | AGTGATGCCTTTGAGGGGAAGGAGGAGCCCCTCTTTCTCCC |
| | ATGCTGCACT |
| | (SEQ ID NO: 485) |
| PGR | CTGTCATTATGGTGTCCTTACCTGTGGGAGCTGTAAGGTCT |
| | TCTTTAAGAGGGCAATGGAAGGGCAGCACAACTACTTATGT |
| | GCTGGAAGAAATGACTGCATCGTTTGATAAAATCCGCAGAA |
| | AAAACTGCCCAGCATGTCGCCTTAGAAAGTGCTGTCAGGCT |
| | GGCATGGTCCTTGGAGGTCGAAAATTTAAAAAGTTCAATAA |
| | AGTCAGAGTTGTGAGAGCACTGGATGCTGTTGCTCTCCCAC |
| | AGCCAGTGGGCGTTCCAAATGAAAGCCAAGCCCTAAGCCAG |
| | AGATTCACTTTTTCACCAGGTCAAGACATACAGTTGATTCC |
| | ACCACTGATCAACCTGTTAATGAGCATTGAACCAGATGTGA |
| | TCTATGCAGGACATGACAACACAAAACCTGACACCTCCAGT |
| | TCTTTGCTGACA |
| | (SEQ ID NO: 486) |
| TFF1 | GGAGCAGAGAGGAGGCAATGGCCACCATGGAGAACAAGGTG |
| | ATCTGCGCCCTGGTCCTGGTGTCCATGCTGGCCCTCGGCAC |
| | CCTGGCCGAGGCCCAGACAGAGACGTGTACAGTGGCCCCCC |
| | GTGAAAGACAGAATTGTGGTTTTCCTGGTGTCACGCCCTCC |
| | CAGTGTGCAAATAAGGGCTGCTGTTTCGACGACACCGTTCG |
| | TGGGGTCCCCTGGTGCTTCTATCCTAATACCATCGACGTCC |
| | CTCCAGAAGAGGAGTGTGAATTTTAGACACTTCTGCAGGGA |
| | TCTGCCTGCATCCTGACGGGGTGCCGTCCCCAGCACGGTGA |
| | TTAGTCCCAGAGCTCGGCTGCCACCTCCACCGGACACCTCA |
| | GACACGCTTCTGCAGCTGTGCCTCGGCTCACAACACAGATT |
| | GACTGCTCTGACTTTGAC |
| | (SEQ ID NO: 487) |
| ERBB2 | CTCCACCCAGCACCTTCAAAGGGACACCTACGGCAGAGAAC |
| | CCAGAGTACCTGGGTCTGGACGTGCCAGTGTGAACCAGAAG |
| | GCCAAGTGCGCAGAAGCCCTGATGTGTCCTCAGGGAGCAGG |
| | GAAGGCCTGACTTCTGCTGGCATCAAGAGGTGGGAGGGCCC |
| | TCCGACCACTTCCAGGGGAACCTGCCATGCCAGGAACCTGT |
| | CCTAAGGAACCTTCCTTCCTGCTTGAGTTCCCAGATGGCTG |
| | GAAGGGGTCCAGCCTCGTTGGAAGAGGAACAGCACTGGGGA |
| | GTCTTTGTGGATTCTGAGGCCCTGCCCAATGAGACTCTAGG |
| | GTCCAGTGGATGCCACAGCCCAGCTTGGCCCTTTCCTTCCA |
| | GATCCTGGGTACTGAAAGCCTTA |
| | (SEQ ID NO: 488) |
| XBP1 | TTGACTATTACACTGCCTGGAGGATAGCAGAGAAGCCTGTC |
| | TGTACTTCATTCAAAAAGCCAAAATAGAGAGTATACAGTCC |
| | TAGAGAATTCCTCTATTTGTTCAGATCTCATAGATGACCCC |
| | CAGGTATTGTCTTTTGACATCCAGCAGTCCAAGGTATTGAG |
| | ACATATTACTGGAAGTAAGAAATATTACTATAATTGAGAAC |
| | TACAGCTTTTAAGATTGTACTTTTATCTTAAAAGGGTGGTA |
| | GTTTTCCCTAAAATACTTATTATGTAAGGGTCATTAGACAA |
| | ATGTCTTGAAGTAGACATGGAATTTATGAATGGTTCTTTAT |
| | CATTTCTCTTCCCCCTTTTTGGCATCCTGGCTTGCCTCCAG |
| | TTTTAGGTCCTTTAGTTTGCTTCTGTAAGCAACGGGAACAC |
| | (SEQ ID NO: 489) |
| K-ALPHA-1 | GCCAACCAGATGGTGAAATGTGACCCTGGCCATGGTAAATA |
| | CATGGCTTGCTGCCTGTTGTACCGTGGTGACGTGGTTCCCA |
| | AAGATGTCAATGCTGCCATTGCCACCATCAAAACCAAGCGC |
| | ACGATCCAGTTTGTGGATTGGTGCCCCACTGGCTTCAAGGT |
| | TGGCATCAACTACCAGCCTCCCACTGTGGTGCCTGGTGGAG |
| | ACCTGGCCAAGGTACAGAGAGCTGTGTGCATGCTGAGCAAC |
| | ACCACAGCCATTGCTGAGGCCTGGGCTCGCCTGGACCACAA |
| | GTTTGACCTGATGTATGCCAAGCGTGCCTTTGTTCACTGGT |
| | ACGTGGGTGAGGGGATGGAGGAAGGCGAGTTTTCAGAGGCC |
| | CGTGAAGATATGGCTGCCCTTGAGAAGGATTATGAGGAGGT |
| | TGGTGTGGATTCTGTTGAAGGAGAGGGTGAGGAAGAAGGAG |
| | AGGAATACTA |
| | (SEQ ID NO: 490) |
| MDH1 | CGCTGCTGTCATCAAGGCTCGAAAACTATCCAGTGCCATGT |
| | CTGCTGCAAAAGCCATCTGTGACCACGTCAGGGACATCTGG |
| | TTTGGAACCCCAGAGGGAGAGTTTGTGTCCATGGGTGTTAT |
| | CTCTGATGGCAACTCCTATGGTGTTCCTGATGATCTGCTCT |
| | ACTCATTCCCTGTTGTAATCAAGAATAAGACCTGGAAGTTT |
| | GTTGAAGGTCTCCCTATTAATGATTTCTCACGTGAGAAGAT |
| | GGATCTTACTGCAAAGGAACTGACAGAAGAAAAAGAAAGTG |
| | CTTTTGAATTTCTTTCCTCTGCCTGACTAGACAATGATGTT |
| | ACTAAATGCTTCAAAGCTGAAGAATCTAAATGTCGTCTTTG |
| | ACTCAAGTACCAAATAATAATAATGCTATACTTAAATTACT |
| | TGTGAAAAACAACACATTTTAAAGATTACGTGCTTCTTGGT |
| | ACAGGTTTGTGAATGACAGTTTATCGTCATGCTGTTAGTG |
| | (SEQ ID NO: 491) |
| HK1 | CGTGTGAAGTGTAGTGGCATCCATTTCTAATGTATGCATTC |
| | ATCCAACAGAGTTATTTATTGGCTGGAGATGGAAAATCACA |
| | CCACCTGACAGGCCTTCTGGGCCTCCAAAGCCCATCCTTGG |
| | GGTTCCCCCTCCCTGTGTGAAATGTATTATCACCAGCAGAC |
| | ACTGCCGGGCCTCCCTCCCGGGGGCACTGCCTGAAGGCGAG |
| | TGTGGGCATAGCATTAGCTGCTTCCTCCCCTCCTGGCACCC |
| | ACTGTGGCCTGGCATCGCATCGTGGTGTGTCAATGCCACAA |
| | AATCGTGTGTCCGTGGAACCAGTCCTAGCCGCGTGTGACAG |
| | TCTTGCATTCTGTTTGTCTCGTGGGGGGAGGTGGACAGTCC |
| | TGCGGAAATGTGTCTTGTCTTCCATTTGGATAAAAGGAACC |
| | AACCAACAAACAATGCC |
| | (SEQ ID NO: 492) |

Hybridization

For each condition, 20 μl of biotin labelled cDNA were denaturated for 2 min. at 99° C., directly placed on ice for 5 min and then added to the hybridization mix (Eppendorf, Hamburg, Germany) to a final volume of 100 μl. The hybridization mix was injected slowly by the injection port of the hybridization frame of the DualChip. The frame was sealed with an aluminium pad and immediately after the sealing, the slides were placed in the Thermomixer comfort (Eppendorf) and incubated overnight (for 12-16 h) at 60° C.

After the hybridization step, the slides were washed 4 times for 2 min with a washing buffer as described in the DualChip Manual.

Fluorescence Detection

The slides were incubated 45 min at room temperature with the Cy3-conjugated IgG Anti-biotin (Jackson Immuno Research Laboratories, Inc #200-162-096) diluted 1/1000× Conjugate-Cy3 in the blocking buffer and protected from light. After this incubation, the slides were washed 5 times for 2 min with the washing buffer and 2 times with distilled water for 2 min and then these slides were dried before being stored at room temperature. The detection was performed in a confocal laser scanner “Autoloader ScanArray” (Packard, USA) and quantified by a specific quantification software. The signal intensity for each spot was corrected by the subtraction of the local background and then averaged. The quantification process was described in detail by de Longueville et al. (2002 Biochem. Pharmacol. 64:137-149).

Results

The signal intensities of hybridization on DualChip human breast cancer for the different amplified gene transcripts are given in the table below. The scale of the scanner is from 1 to 65536.

### Signal Intensities of 14 Amplified Gene Transcripts after Hybridization on DualChip Human Breast Cancer. ND means not Detectable Signal

| | ER negative | ER positive |
|---|---|---|
| BCL2 | ND | 4163 |
| CCNE1 | 106 | 860 |
| ERBB2 | ND | ND |
| ESR1 | 1178 | 59446 |
| GATA3 | 26 | 16504 |
| MCM7 | 3375 | 1429 |
| MKI67 | 3076 | 2104 |
| PGR | ND | 7039 |
| SLC39A6 | 2425 | 12329 |
| TFF1 | ND | 60284 |
| xBP1 | ND | 14485 |
| K-ALPHA-1 | 56671 | 43994 |
| HK1 | 3696 | 7611 |
| MDH1 | 334 | ND |

### EXAMPLE 30

### Amplification and Quantification of mRNA Present in Different Concentrations

6 internal standards were selected. They are part of the Dualchip product and the capture probes are present in the predefined DualChip. They were in vitro transcribed polyadenylated RNA which were produced by in vitro transcription using a T7 RNA polymerase. They were quantified and diluted in order to obtain the required concentrations. For each of them a primer pair was designed having a specific sequence complementary either of the sense and the other one of the antisense strand. Each of the different primers had an additional 5′ end universal sequence being; TGCTATGCTCACAGATGCGA (SEQ ID NO: 460) which was also the universal primer.

The specific part of the primers sequences for the different genes are described in the table below. We also provide the concentrations of the various mRNA incorporated into the Primer sequences of the six internal standards.

| | | | |
|---|---|---|---|
| | | | Concentrations |
| Genes | Sense primer (5′ -> 3′) | Antisense primer (5′ -> 3′) | (picomoles) |

| | | | |
|---|---|---|---|
| Rbcs | TTGCACCTTTCACTGGTCTCAA | GCTAGCAATGGAAGTGATGTCAA | 10000 |
| | (SEQ ID NO: 461) | (SEQ ID NO: 462) | |
| Cab | ACTGCTGAGAACTTTGCCAACTT | CCAGCTAATGCCAAAGGATCA | 1000 |
| | (SEQ ID NO: 463) | (SEQ ID NO: 464) | |
| Rbcl | AAAGGGCATTACTTGAATGCTACTG | GAACGCCCAATTCTCTAGCAAA | 100 |
| | (SEQ ID NO: 465) | (SEQ ID NO: 466) | |
| Rca | ACTGGTAACGACTTCTCCACATTG | TCCCTAGTTGGTGCCCAGTAGA | 30 |
| | (SEQ ID NO: 467) | (SEQ ID NO: 468) | |
| Tapg | GGTGTAAAGATAAGTGATGTAACGTATGAA | TCCACTACATGGATTTGTTTTGCT | 10 |
| | (SEQ ID NO: 469) | (SEQ ID NO: 470) | |
| Sip | TGTTCAATTCAGGAGGAGCAATC | CGCGAACCTTCATGGAAACT | 5 |
| | (SEQ ID NO: 471) | (SEQ ID NO: 472) | |

The experiments of RT-PCR, hybridization and fluorescence detection and quantification were conducted as described in Example 27. The detection of the amplified targets was performed on the DualChip human breast (Eppendorf, Hamburg, Germany). The DualChips are obtained by spotting aminated capture molecules on aldehyde activated glass obtained according to the EP01313677B1 using a home made robotic device. The capture molecules were part of an Xmer technology of Eppendorf and are between 200 and 450 bp long. The spots were around 250 μm in diameter. The slides were stored at 4° C.

The capture probes for the different genes detected in this example are presented in the table below. Their sequences are complementary of the gene transcripts which have to be detected. The sequence complementary of the amplified target sequence is shown in bold. The sequence located in the 5′ end of the capture molecules serves as spacer for the binding of the target amplified sequences.

Capture probe sequence for the different detected genes. The sequence complementary of the amplified target sequence is shown in bold.

| | |
|---|---|
| Genes | Capture Probe |

| | |
|---|---|
| Rbcs | CACACGCAGCAATGTTACACAAGCTAGCATGGTTGCACCTTTCA |
| | CTGGTCTCAAATCTTCAGCCACTTTCCCTGTTACAAAGAAGCAA |
| | AACCTTGACATCACTTCCATTGCTAGCAATGGTGGAAGAGTTAG |
| | CTGCATGCAGGTGTGGCCACCTATTAACATGAAGAAGTACGAGA |
| | CACTCTCATACCTTCCCGATTTGTCCGACGAGCAATTGCTTAGT |
| | GAAATTGAGTACCTTTTGAAAAATGGATGGGTTCCTTGCTTGGA |
| | ATTTGAGACTGAGCACGGATTTGTCTACCGTGAGAACAACAAGT |
| | CACCAGGATACTATGATGGAAGGTACTGGACCATGTGGAAGTTG |
| | CCTATGTTTGGGTGCACTGATGCAACCCAAGTGTTGGCTGAGGT |
| | TCAAGAGGCTAAAAAGGCATACCCACAAGCATGGGTCAGAATCA |
| | TTGGATTCGACAATGTGCGTCAAGTGCAGTGTATCAGTTTCATT |
| | GCCTACA |
| | (SEQ ID NO: 473) |
| Cab | TTGTTGGTCAAGCCTGGAGTGGCATTCCATGGTTTGAGGCTGGT |
| | GCTGATCCTGGCGCTATTGCACCTTTCTCTTTCGGCTCACTTCT |
| | TGGCACTCAGCTTCTCCTCATGGGTTGGGTTGAGAGCAAAAGGT |
| | GGGTCGACTTCTTCGACAATGACTCTCAGTCTATAGATTGGGCC |
| | ACTCCATGGTCCAAGACTGCTGAGAACTTTGCCAACTTCACAGG |
| | CGAACAGGGTTACCCTGGTGGCAAATTCTTTGATCCTTTGGCAT |
| | TAGCTGGTACACTTAACAATGGAGTTTACGTCCCTGACACAGAG |
| | AAGCTTGAGAGATTAAAGCTTGCTGAGATCAAGCATTCTAGACT |
| | TGCTATGTTAGCCATGTTAATTTTCTATTTTGAGGCTGGACAAG |
| | GGAAGACA |
| | (SEQ ID NO: 474) |
| Rbcl | AACTCACAACCATTTATGCGTTGGAGAGATCGTTTCTTATTTTG |
| | TGCCGAAGCACTTTWTAAAGCACAGACTGAAACAGGTGAAATCA |
| | AAGGGCATTACTTGAATGCTACTGCAGGTACATGCGAAGAAATG |
| | ATCAAAAGAGCTGTATTTGCTAGAGAATTGGGCGTTCCGATCGT |
| | AATGCATGACTACTTAACGGGGGGATTTACCGCAAATAATGCAT |
| | GCGGTTATTGATAGACAGAAGAATCATGGTATCCACTTCCGGGT |
| | ATTAGCAAAAGCGTTACGTATGTCTGGTGGAGATCATATTCACT |
| | CTGGTACCGTAGTAGGTAAACTTGAAGGTGAAAGAGACATAACT |
| | TTGGGCTTTGTTGA |
| | (SEQ ID NO: 475) |
| Rca | GCAAAGGTACAGAGAGGCAGCTGAAATCATCAGGAAAGGAAACA |
| | TGTGTTGTCTCTTCATCAACGATCTCGATGCAGGAGCTGGTAGA |
| | ATGGGTGGAACTACCCAATACACCGTCAACAACCAGATGGTGAA |
| | TGCCACCCTCATGAACATTGCTGACAACCCAACAAATGTCCAGC |
| | TCCCCGGTATGTACAACAAGCAAGAGAACGCCAGGGTACCCATT |
| | ATTGTCACTGGTAACGACTTCTCCACATTGTATGCTCCTCTTAT |
| | CCGTGATGGTCGTATGGAGAAGTTCTACTGGGCACCAACTAGGG |
| | AGGATAGAATTGGTGTTTGCAAGGGTATTTTCAGAACTGACAAC |
| | GTCCCTGAGGAAGCTGTTGTAAAGATTGTCGATTCCTTCCCTGG |
| | ACAATCTATTGATTTCTTTGGTGCTTTGAG |
| | (SEQ ID NO: 476) |
| Tapg | AAATGTGACAGTTAAGATGGTTAGCTTCACAAGTACTGAGAATG |
| | GTGTGAGAGTAAAAACATGGGCAAGACCTAGCAATGGTTTTGTT |
| | AGAAATGTTTTATTTCAACATATTGTTATGAGTAATGTTCAAAA |
| | TCCAATAATCATAGATCAAAATTATTGTCCTAATCATGAAAGTT |
| | GTCCTAATCAGGGCTCAGGTGTAAAGATAAGTGATGTAACGTAT |
| | GAAGACATACATGGAACATCAGCTACAGAAATCGCGGTGAAATT |
| | AGATTGTAGCAAAACAAATCCATGTAGTGGAATAACACTTGAAG |
| | ATGTGAATCTTAGTTATAAAAATGGTAGAGCTGAAGCTTCATGT |
| | GTTAATGCTGGAGGAAGAGCTTCTGGTTTTGAAGAACTTAGTAA |
| | ATGCTT |
| | (SEQ ID NO: 477) |
| Sip | TGATGTCAATTACTTACCAAGGATTGCTCATGATGGATGGACCG |
| | GCGATGCCATTCTGTATTCTCATCTTCATAGGGAATTGATCAAT |
| | CTTCCTAAAAATACTTCGATTCCAATCACTCTAAACGCGAGAGA |
| | ATATGAAGTCTTTACAGTTGTTCCAATCAATGAAATGTNTACAG |
| | GATCAAGATTTGCTCCGATTGGTCTTGTGAATATGTTCAATTCA |
| | GGAGGAGCAATCAAAGAGGTGAAATATGAAACAGAGGGAAAATG |
| | TGGACTAGTTTCCATGAAGGTTCGCGGATGTGGAACGTTTGGAG |
| | CTTATTCATCTGGGAAGCCTAAACGAATTCATGTTGACAACGAA |
| | GAAGTACAGTTTGATTACGACGAATCCTCTGGATTGTTCACCAT |
| | TAACATTACAGTTCCTGAT |
| | (SEQ ID NO: 478) |

### EXAMPLE 31

### Amplification and Detection of Different Bacteria Species

The bacteria to be detected, the genes to be amplified, the primer pairs and the amplification conditions are as in Example 26. The universal primers were biotinylated at the 5′ terminus.

The capture molecules had the same sequences as the probes of example 26 with an amino group at the 5′ end. The beads were the xMAP Multi-analyte COOH Microsperes from Luminex (Oosterhout, The Nederlands). The beads were labelled with fluorescent dyes and contained surface layer of avidin which were used for the binding of the biotinylated-probes. The beads were obtained at a concentration of 2.5×10⁶ beads per ml. One capture probe was bound to one particular bead population. The coupling of the probes on their respective beads was performed as proposed by Cowan, L. et al. (2004 J. Clin Microbiol. 42:474-477).

To couple the probes to the microspheres (Luminex Corp.), 200 μmol of polynucleotides probes, 2.5×10⁶ microspheres, and 25 μg of freshly purchased N-(3-dimethylaminopropyl)-N′-ethylcarbodiimide (Pierce Chemical, Rockford, Ill.) were combined in 25 μl of 100 mM 2-(N-morpholino)ethanesulfonic acid (MES), pH 4.5 (Sigma, St. Louis, Mo.). The reaction mixtures were incubated at room temperature in the dark for 30 min. The N-(3-dimethylaminopropyl)-N′-ethylcarbodiimide addition and subsequent incubation were repeated once. After coupling, the microspheres were washed with 0.5 ml of 0.02% Tween 20 followed by 0.5 ml of 0.1% sodium dodecyl sulfate. The prepared microspheres were suspended in 50 μl of Tris-EDTA, pH 8.0, and stored at 4° C. in the dark. A microspheres mix was prepared by combining equal volumes of each of the different beads bearing the different capture molecules.

For hybridization, the amplicons were first denatured by preparation of a hybridization mix containing 5 μl of PCR product, 5 μl of sensihyb solution (Eppendorf, Hamburg, Germany), 4 μl of hybridization control (Eppendorf, Hamburg, Germany) and 6 μl of water. 5 μl of NaOH was then added to the mix and then incubated 5 min at room temperature.

The microsphere mix was prepared by dilution of the microsphere mix in the hybridisation solution (Eppendorf, Hamburg, Germany) to a final concentration of approximately 150 microspheres of each set/μl. PCR product (25 μl) and diluted microsphere mix (25 μl) were combined in a Thermowell 96-well plate (VWR International, West Chester, Pa.). The reaction mixtures were incubated for 60 min at 60° C., in a GeneAmp 9700 PCR System (Perkin-Elmer, Foster City, Calif.). The plate is centrifuged at 2,250×g for 3 min, the supernatant was removed by pipette, and the microspheres were resuspended with 75 μl of detection buffer (R-phycoerythrin-conjugated streptavidin [Molecular Probes, Eugene, Oreg.] diluted to 4 μg/ml with 1× hybridization buffer). Following 5-min incubation at 52° C., the samples were analyzed in the Luminex 100, version 1.7; a minimum of 100 events/microsphere set were analyzed.

The beads were then analyzed in a Luminex 100 IS system (Oosterhout, The Nederlands) which was a flow cell fluorometry which detected both the beads according to their fluorescent dyes but also the fluorochrome attached to the labelled targets by the use of two different lasers. The Luminex 100 system associated the presence of a specific capture probe present on a bead with a particular dye with the intensity of the fluorochrome associated with the binding of the target on this capture molecule. The quantification was performed as presented by Spiro A. and M. Lowe (2002 Appl. Environ. Microbiol. 68:1010-1013). The intensity values of the target signals (reporter signals) were converted into units known as molecules of equivalent soluble fluorochrome (MESF) using Quantum 27 (R-PE) Reference Standards (Bangs Laboratories, Inc.) according to standard procedures. Cytometry data were analyzed with FCS Express version 1.065 (De Novo Software). The mean intensity (Iₛ) of the reporter signal and intersample standard deviation (SD) were determined by running ≧7 replicate tubes. A similar procedure was used for the background signal (I_{b}). The uncertainty in the fluorescence response F=Iₛ−I_{b} was calculated using the standard error SD in the difference of means.

TABLE 3
Fish families

| | Family | | | | | |
|---|---|---|---|---|---|---|
| Classification | Scombridae | Salmonidae | Merlucciidae | Pleuronectidae | Gadidae | Clupeidae |
| Genera | Auxis | Oncorhynchus | Merluccius | Pleuronectes | Pollachius | Sardina |
| Species | A. thazard | O. mykiss | M. merluccius | P. platessus | P. virens | S. pilchardus |
| Genera | Sarda | Salmo | | Platichthys | Gadus | |
| Species | S. sarda | S. salar | | P. flesus | G. morhua | |
| | | S. trutta | | | G. macrocephalus | |
| Genera | Scomber | | | Reinhardtius | | |
| Species | S. scombrus | | | R. hippoglossoides | | |
| Genera | Thunnus | | | | | |
| Species | T. albacares | | | | | |
| | T. obesus | | | | | |
| | T. alalunga | | | | | |
| | T. thynnus | | | | | |

| Animal Meat | | | | |
|---|---|---|---|---|
| | Family | | | |
| Classification | Galinacea | Leporidae | Suidae | Bovidae |
| Genera | Chicken | Rabbit | Pig | |
| Genera | Duck | | Wild pig | |
| Genera | Ostrich | | | |
| Genera | Turkey | | | |
| Genera | Quail | | | Cow |
| Species | | | | Brownswiss, Jersey, Hereford, |
| | | | | Simmental, Piemontaise, Canadienne, |
| | | | | RedAngus, Limousine, AberdeenAngus, |
| | | | | Butana, Charolais, Fresian, Kenana, |
| | | | | N'Dama |

What is claimed is:

1. A method for identifying and/or quantifying an organism or part of an organism in a sample by detecting a nucleotide sequence specific for said organism, among at least 4 other sequences from other organisms or from parts of the organisms comprising the steps of:
producing derived sequences from said organism nucleotide sequences by incorporation of at least one common sequence in said organism nucleotide sequences in order to obtain a partial homology between the said specific derived nucleotide sequences;
amplifying said specific derived nucleotide sequences by PCR into double stranded target nucleotide sequences using a unique pair of primers, which recognize the common sequence of the derived sequences and which are capable of amplifying at least 4 of said other derived nucleotide sequences as to produce full-length target nucleotide sequences having between about 60 and about 800 bases;
contacting said full length target nucleotide sequences resulting from the amplifying step with at least 5 different single-stranded capture nucleotide sequences having between about 55 and about 800 bases, said single-stranded capture being covalently bound in an microarray to insoluble solid support(s) and said capture nucleotide sequences comprising a nucleotide sequence of at least 15 bases which is able to specifically bind to said full-length target nucleotide sequence without binding to said at least 4 other derived nucleotide sequences, and said specific sequence is separated from the surface of the solid support by a spacer comprising a nucleotide sequence of at least 40 bases in length; and
detecting specific hybridization of said target nucleotide sequence to said capture nucleotide sequences.

2. The method of claim 1, wherein the production of the derived sequences is obtained by amplification of the nucleotide sequences present in the sample using specific primer pairs, each member of said primer pair comprises a sequence complementary to one of the two strands of a given polynucleotide sequence and a common sequence (U) serving as an universal amplifying sequence, being identical for all the specific primers and being located at the 5′ end of the primers and further amplification with a unique primer which recognize the common sequence.

3. The method of claim 2, wherein the two members of a given primer pair have two different common sequences (U1 and U2) which are used as two universal amplifying sequences, and further amplified by a unique pair of primers which recognize the two common sequences.

4. The method of claim 2, wherein the length of the sequence complementary to one of the two strands of a given polynucleotide sequence of the specific primer pair is selected from the group consisting of at least 6, and at least 15 nucleotides.

5. The method of claim 4 wherein the sequences complementary to the strands of the polynucleotide sequence of the specific primer pairs show a homology of lower than about 30%.

6. The method of claim 2, wherein U is at least 15 nucleotides in length.

7. The method of claim 1, wherein the nucleotide sequences of the sample to be detected have less than 30% homology to each other.

8. The method of claim 1, wherein the amplified homologous original nucleotide sequences are mRNA first reverse transcribed into cDNA with the same primer.

9. The method of claim 1, wherein said capture nucleotide sequence is bound to the insoluble solid support at a specific location according to an array, said array having a density of at least 4 different bound single stranded capture nucleotide sequences/cm² of solid support surface.

10. The method of claim 1, wherein the specific sequence of the capture nucleotide sequence, able to hybridize with their corresponding target nucleotide sequence comprise between about 15 and about 40 continuous nucleotide sequence complementary to one of the two strands of the amplified target sequences.

11. The method of claim 1, wherein the binding of the amplicons on the capture probe is such as to produce two non complementary ends, one being a spacer end and the other one a non-spacer end, such that the spacer end is non-complementary to the spacer portion of the capture molecule and said spacer end exceeds said non-spacer end by at least 50 bases.

12. The method of claim 11, wherein the density of the capture nucleotide sequence bound to the surface at a specific location is higher than 100 fmoles per cm² of solid support surface.

13. The method of claim 1, wherein the quantification of the organism present in the biological sample is obtained by the quantification of the signal present at a particular location of the support.

14. The method of claim 1, wherein the production of the derived sequences from the organism nucleotide sequences of the sample and the amplification by the universal primers are performed in one amplification process.

15. The method of claim 1, wherein the primers specific of the targets are at a concentration lower than 50 nM.

16. The method of claim 15, wherein the concentration of the universal primers is at least 500 nM.

17. The method of claim 1, wherein the ratio between the concentration of universal primers and the concentration of the specific target primers in the amplification PCR solution is at least 20.

18. The method of claim 17, wherein the total concentration of the overall specific primers does not exceed 1000.

19. The method of claim 1, wherein the universal primers have a Tm ±5° C. and better ±2° C. of the primers specific for the sample nucleotide sequences.

20. The method of claim 1, wherein the annealing temperature of the PCR cycles is at least 5° C. lower than the Tm of the specific and the universal primers.

21. The method of claim 1 wherein the PCR amplification is obtained with less than 25 cycles.

22. The method of claim 1, wherein the concentration ratio between two different polynucleotide target sequences being detected is higher than 10.

23. The method of claim 1, wherein the amplification (PCR) solution comprises at least 20 different target specific primers.

24. The method of claim 1, wherein the ratio between the concentrations of the two universal primers in the amplification solution is comprised between 1.2 and 2.

25. The method of claim 1, wherein the PCR amplification is performed by a DNA polymerase being a hot-start DNA polymerase.

26. The method of claim 1, wherein the PCR amplification is performed by a DNA polymerase being a Topo Taq DNA polymerase.

27. The method of claim 1, wherein the insoluble solid support is in the form of a multiwell plate.

28. The method of claim 1, wherein the different capture molecules are immobilized on different beads.

29. The method of claim 28, wherein different beads having different capture molecules are labeled so as to be discriminated from each other.

30. The method of claim 1, wherein the detection and/or the quantification of the amplified target sequences is obtained after their hybridization on corresponding capture probes in the amplification solution.

31. The method of claim 1, wherein the amplification and the detection are performed in the same closed device.

32. The method of claim 31, wherein the detection of the amplified sequences is performed during the PCR cycles.

33. The method of claim 62, wherein the amplification is a real time PCR.

34. The method of claim 1, wherein the detection of the presence of pathogenic organisms being or not microorganisms such as bacterial or virus is obtained by the detection of their genomic DNA sequences.

35. The method of claim 1, for the detection of the presence of Genetically Modified Organisms (GMO) by the detection of their genomic DNA sequences.

36. The method of claim 1, for the detection of the presence of mutations or deletions in some specific parts of a genome or in genes.

37. The method of claim 1, wherein the original sequence to be detected and/or quantified in the sample belongs to the cytochrome P450 forms family.

38. The method of claim 1 for the detection and quantification of at least 20 gene transcripts.

39. The method of claim 1, wherein the detection and/or quantification of the nucleotide sequence is performed on degraded RNA extracted from the paraffin embedded tissue.

40. The method of claim 1, wherein the detection and/or quantification of the nucleotide sequence is performed on target amplified cDNA having a full length of between about 50 and about 150 bases long.

41. The method of claim 1, wherein the full-length target nucleotide sequences are single stranded DNA produced by isothermal amplification.

42. The method of claim 1, wherein the different single-stranded capture nucleotide sequences bound to the support have their entire sequences complementary or identical to one part of the transcript sequence to be detected.

43. A method for identifying and/or quantifying at least 5 transcripts of a cell in a sample comprising the steps of:
producing derived sequences from the parts of the transcript sequences present in the cell extract by incorporation of at least one common sequence in said parts of transcript sequences in order to obtain a partial homology between the said derived nucleotide sequences;
amplifying said derived nucleotide sequences as to produce full-length target nucleotide sequences having between 60 and 800 bases;
contacting said full-length target nucleotide sequences resulting from the amplifying step with at least 5 different single-stranded capture nucleotide sequences having between about 55 and about 800 bases, said single-stranded capture nucleotide sequences being covalently bound in an microarray to insoluble solid support(s) and said capture nucleotide sequences comprising a nucleotide sequence of at least 15 bases which is able to specifically bind to said full-length target nucleotide sequence, and said specific sequence is separated from the surface of the solid support by a nucleotide sequence of at least 40 bases in length; and
detecting specific hybridization of said target nucleotide sequence to said capture nucleotide sequences and quantifying the transcript expression level in the cell.

44. The method of claim 43, wherein said at least 5 different single-stranded capture nucleotide sequences have between about 200 and about 450 bases in length.

45. The method of claim 43 for the detection and quantification of at least 20 gene transcripts.

46. The method of claim 43, wherein the detection and/or quantification of the nucleotide sequence is performed on degraded RNA extracted from the paraffin embedded tissue.

47. The method of claim 43, wherein the detection and/or quantification of the nucleotide sequence is performed on target amplified cDNA having a full length of between about 50 and about 150 bases long.

48. The method of claim 43, wherein the full-length target nucleotide sequences are double stranded DNA produced by PCR.

49. The method of claim 43, wherein the full-length target nucleotide sequences are single stranded DNA produced by isothermal amplification.

50. The method of claim 43, wherein the different single-stranded capture nucleotide sequences bound to the support have their entire sequences complementary or identical to one part of the transcript sequence to be detected.

51. A diagnostic and/or quantification kit which comprises
an insoluble solid support upon which single stranded capture nucleotide sequences are bound in an array, said single stranded capture nucleotide sequences containing a sequence of between about 10 and about 600 bases specific for a target nucleotide sequence to be detected and/or quantified and having a total length comprised between about 30 and about 800 bases comprising a spacer having a nucleotide sequence of at least 40 bases, said single stranded capture nucleotide sequences being disposed upon the surface of the solid support and
an amplification (PCR) solution that comprises at least 5 different target specific primers and a universal primer pair, a thermostable DNA polymerase, a plurality of dNTPs and a buffered solution having a pH comprised between 7 and 9 for containing the primers.

52. The kit according to the claim 51, wherein said single stranded capture nucleotide sequences contains a sequence of between about 50 and about 450 bases specific for a target nucleotide sequence to be detected and/or quantified.

53. The kit according to the claim 51, comprising a device having a chamber for performing the amplification reaction together with detection and possibly a quantification of amplified target sequences.

54. The diagnostic kit according to claim 51, wherein the insoluble solid support is in the form of a multiwell plate.

55. The diagnostic kit according to claim 51, wherein the insoluble solid support is a series of beads.

56. The diagnostic kit according to claim 51, wherein the capture nucleotide sequences are specific to a target nucleotide sequence to be detected and/or quantified which is specific for a gene selected from the group consisting of bacterial genes, human genes, cytochrome P450 family genes.

57. The diagnostic kit according to claim 51, comprising biochips, for identification and/or quantification of 5 GMO obtained after amplification of one of their DNA sequences with specific primers and detection on specific capture molecules present on an array.

58. The kit according to claim 51, wherein the capture molecules present on an array contain at least 5 bases located on either sides of the 3′ or 5′ flanking regions of the foreign DNA incorporated into the genome of the plant in order to obtain a of the GMO.

59. The diagnostic kit according to claim 51, further comprising biochips for identification and/or quantification of 5 bacteria species obtained after amplification of one of their DNA sequences with specific primers and universal primer(s) and detection on an array.

60. The diagnostic kit according to claim 51, further comprising biochips for identification and/or quantification of different SNP located at different locations in the genome of an organism.


SEQUENCE LISTING (492 sequences)

SEQ ID NO: 1 - DNA, 23 bp, Artificial Sequence
  primer for amplification of S. aureus
cttttgctgatcgtgatgacaaa

SEQ ID NO: 2 - DNA, 25 bp, Artificial Sequence
  primer for amplification of S. aureus
tttatttaaaatatcacgctcttcg

SEQ ID NO: 3 - DNA, 23 bp, Artificial Sequence
  primer for amplification of S. epidermidis
tcgcggtccagtaatagattata

SEQ ID NO: 4 - DNA, 22 bp, Artificial Sequence
  primer for amplification of S. epidermidis
tgcatttccagttatttctccc

SEQ ID NO: 5 - DNA, 24 bp, Artificial Sequence
  primer for amplification of S. haemolyticus
attgatcatggtattgatagatac

SEQ ID NO: 6 - DNA, 25 bp, Artificial Sequence
  primer for amplification of S. haemolyticus
tttaatctttttgagtgtcttatac

SEQ ID NO: 7 - DNA, 25 bp, Artificial Sequence
  primer for amplification of S. saprophyticus
taaaatgaaacaactcggttataag

SEQ ID NO: 8 - DNA, 24 bp, Artificial Sequence
  primer for amplification of S. saprophyticus
aaactatccataccattaagtacg

SEQ ID NO: 9 - DNA, 24 bp, Artificial Sequence
  primer for amplification of S. hominis
cgaccagataacaaaaaagcacaa

SEQ ID NO: 10 - DNA, 22 bp, Artificial Sequence
  primer for amplification of S. hominis
gtaattcgttaccatgttctaa

SEQ ID NO: 11 - DNA, 27 bp, Artificial Sequence
  capture nucleotide ATaur02
atttaaaatatcacgctcttcgtttag

SEQ ID NO: 12 - DNA, 27 bp, Artificial Sequence
  capture nucleotide ATepi02
attaagcacatttctttcattatttag

SEQ ID NO: 13 - DNA, 27 bp, Artificial Sequence
  capture nucleotide AThae02
atttaaagtttcacgttcattttgtaa

SEQ ID NO: 14 - DNA, 27 bp, Artificial Sequence
  capture nucleotide AThom02
atttaatgtctgacgttctgcatgaag

SEQ ID NO: 15 - DNA, 27 bp, Artificial Sequence
  capture nucleotide ATsap02
acttaatacttcgcgttcagcctttaa

SEQ ID NO: 16 - DNA, 24 bp, Artificial Sequence
  consensus primer APstap03
cccactcgcttatatagaatttga

SEQ ID NO: 17 - DNA, 23 bp, Artificial Sequence
  consensus primer APstap04
ccactagcgtacatcaattttga

SEQ ID NO: 18 - DNA, 25 bp, Artificial Sequence
  consensus primer APstap05
ggtttaataaagtcaccaacatatt

SEQ ID NO: 19 - DNA, 47 bp, Artificial Sequence
  capture nucleotide (with spacer sequence) ATepi03
  misc_feature (1)...(20): spacer sequence
gaattcaaagttgctgagaaattaagcacatttctttcattatttag

SEQ ID NO: 20 - DNA, 67 bp, Artificial Sequence
  capture nucleotide (with spacer sequence) ATepi04
  misc_feature (1)...(40): spacer sequence
gaattcaaagttgctgagaatagttcaatggaaggaagcgattaagcacatttctttcattatttag

SEQ ID NO: 21 - DNA, 87 bp, Artificial Sequence
  capture nucleotide (with spacer sequence) ATepi05
  misc_feature (1)...(60): spacer sequence
gaattcaaagttgctgagaatagttcaatggaaggaagcgtcttcttaaaatctaaagaaattaagcacatttctttcattatttag

SEQ ID NO: 22 - DNA, 67 bp, Artificial Sequence
  capture nucleotide (with spacer sequence) Ataur27
  misc_feature (1)...(40): spacer sequence
gaattcaaagttgctgagaatagttcaatggaaggaagcgatttaaaatatcacgctcttcgtttag

SEQ ID NO: 23 - DNA, 67 bp, Artificial Sequence; identical to SEQ ID NO: 20
  capture nucleotide (with spacer sequence) Atepi27
  misc_feature (1)...(40): spacer sequence

SEQ ID NO: 24 - DNA, 67 bp, Artificial Sequence
  capture nucleotide (with spacer sequence) Athae27
  misc_feature (1)...(40): spacer sequence
gaattcaaagttgctgagaatagttcaatggaaggaagcgatttaaagtttcacgttcattttgtaa

SEQ ID NO: 25 - DNA, 67 bp, Artificial Sequence
  capture nucleotide (with spacer sequence) Athom27
  misc_feature (1)...(40): spacer sequence
gaattcaaagttgctgagaatagttcaatggaaggaagcgatttaatgtctgacgttctgcatgaag

SEQ ID NO: 26 - DNA, 67 bp, Artificial Sequence
  capture nucleotide (with spacer sequence) Atsap27
  misc_feature (1)...(40): spacer sequence
gaattcaaagttgctgagaatagttcaatggaaggaagcgacttaatacttcgcgttcagcctttaa

SEQ ID NO: 27 - DNA, 22 bp, Artificial Sequence
  consensus primer APcons3-1
taayaaartcaccaacataytc

SEQ ID NO: 28 - DNA, 22 bp, Artificial Sequence
  consensus primer APcons3-2
  misc_feature (1)...(22): n = A,T,C or G
  misc_feature 5: n = A,T,C or G
  misc_feature 5: n = A,T,C or G
tymgntcatttatggaagatac

SEQ ID NO: 29 - DNA, 67 bp, Artificial Sequence
  capture nucleotide (with spacer sequence) Ataur15
  misc_feature (1)...(52): spacer sequence
gaattcaaagttgctgagaatagttcaatggaaggaagcgtcttcttaaaatgctcttcgtttagtt

SEQ ID NO: 30 - DNA, 67 bp, Artificial Sequence
  capture nucleotide (with spacer sequence) Ataur40
  misc_feature (1)...(27): spacer sequence
gaattcaaagttgctgagaatagttcaaatctttatttaaaatatcacgctcttcgtttagttcttt

SEQ ID NO: 31 - DNA, 67 bp, Artificial Sequence
  capture nucleotide (with spacer sequence) Atana15
  misc_feature (1)...(52): spacer sequence
gaattcaaagttgctgagaatagttcaatggaaggaagcgtcttcttaaaatgctcttcatttagtt

SEQ ID NO: 32 - DNA, 67 bp, Artificial Sequence
  capture nucleotide (with spacer sequence) Atana27
  misc_feature (1)...(40): spacer sequence
gaattcaaagttgctgagaatagttcaatggaaggaagcggtttaaaatatcacgctcttcatttag

SEQ ID NO: 33 - DNA, 67 bp, Artificial Sequence
  capture nucleotide (with spacer sequence) Atana40
  misc_feature (1)...(27): spacer sequence
gaattcaaagttgctgagaatagttcaaatctttgtttaaaatatcacgctcttcatttagttcttt

SEQ ID NO: 34 - DNA, 67 bp, Artificial Sequence
  capture nucleotide (with spacer sequence) Atepi15
  misc_feature (1)...(52): spacer sequence
gaattcaaagttgctgagaatagttcaatggaaggaagcgtcttcttaaaattttcattatttagtt

SEQ ID NO: 35 - DNA, 67 bp, Artificial Sequence
  capture nucleotide (with spacer sequence) Atepi40
  misc_feature (1)...(27): spacer sequence
gaattcaaagttgctgagaatagttcaaatctttattaagcacatttctttcattatttagttcctc

SEQ ID NO: 36 - DNA, 40 bp, Artificial Sequence
  spacer sequence
gaattcaaagttgctgagaatagttcaatggaaggaagcg

SEQ ID NO: 37 - DNA, 27 bp, Artificial Sequence
  S. aureus femA capture sequence
atttaaaatatcacgctcttcgtttag

SEQ ID NO: 38 - DNA, 27 bp, Artificial Sequence
  S. epidermidis femA capture sequence
attaagcacatttctttcattatttag

SEQ ID NO: 39 - DNA, 27 bp, Artificial Sequence
  S. haemolyticus femA capture sequence
atttaaagtttcacgttcattttgtaa

SEQ ID NO: 40 - DNA, 27 bp, Artificial Sequence
  S. hominis femA capture sequence
atttaatgtctgacgttctgcatgaag

SEQ ID NO: 41 - DNA, 27 bp, Artificial Sequence
  S. saprophyticus femA capture sequence
acttaatacttcgcgttcagcctttaa

SEQ ID NO: 42 - DNA, 27 bp, Artificial Sequence
  S. capitis femA capture sequence
attaagaacatctctttcattattaag

SEQ ID NO: 43 - DNA, 24 bp, Artificial Sequence
  S. caseolyticus femA capture sequence
ataaagacattcgagacgaaggct

SEQ ID NO: 44 - DNA, 27 bp, Artificial Sequence
  S. cohnii femA capture sequence
acttaacacttcacgctctgacttgag

SEQ ID NO: 45 - DNA, 27 bp, Artificial Sequence
  S. gallinarum femA capture sequence
acttaaaacttcacgttcagcagtaag

SEQ ID NO: 46 - DNA, 27 bp, Artificial Sequence
  S. intermedius femA capture sequence
gtggaaatcttgctcttcagatttcag

SEQ ID NO: 47 - DNA, 27 bp, Artificial Sequence
  S. lugdunensis femA capture sequence
ttctaaagtttgtcgttcattcgttag

SEQ ID NO: 48 - DNA, 27 bp, Artificial Sequence
  S. schleiferi femA capture sequence
tttaaagtcttgcgcttcagtgttgag

SEQ ID NO: 49 - DNA, 27 bp, Artificial Sequence
  S. sciuri femA capture sequence
gttgtattgttcatgttctttttctaa

SEQ ID NO: 50 - DNA, 27 bp, Artificial Sequence
  S. simulans femA capture sequence
ttctaaattcttttgttcagcgttcaa

SEQ ID NO: 51 - DNA, 27 bp, Artificial Sequence
  S. warneri femA capture sequence
agttaaggtttctttttcattattgag

SEQ ID NO: 52 - DNA, 27 bp, Artificial Sequence
  S. xylosis femA capture sequence
gcttaacacctcacgttgagcttgcaa

SEQ ID NO: 53 - DNA, 20 bp, Artificial Sequence
  consensus primer MycU4 sense
catgcagtgaattagaacgt

SEQ ID NO: 54 - DNA, 18 bp, Artificial Sequence
  consensus primer APmcon02 antisense
gtasgtcatrrstyctcc

SEQ ID NO: 55 - DNA, 21 bp, Artificial Sequence
  Mycobacteria avium capture probe
cggtcgtctccgaagcccgcg

SEQ ID NO: 56 - DNA, 20 bp, Artificial Sequence
  Mycobacteria gastrii 1 capture probe
gatcggcagcggtgccgggg

SEQ ID NO: 57 - DNA, 19 bp, Artificial Sequence
  Mycobacteria gastrii 3 capture probe
gtatcgcgggcggcaaggt

SEQ ID NO: 58 - DNA, 24 bp, Artificial Sequence
  Mycobacteria gastrii 5 capture probe
tctgccgatcggcagcggtgccgg

SEQ ID NO: 59 - DNA, 24 bp, Artificial Sequence
  Mycobacteria gastrii 7 capture probe
gccggggccggtattcgcgggcgg

SEQ ID NO: 60 - DNA, 22 bp, Artificial Sequence
  Mycobacteria gordonae capture probe
gacgggcactagttgtcagagg

SEQ ID NO: 61 - DNA, 21 bp, Artificial Sequence
  Mycobacterium intracellulare 1 capture probe
gggccgccgggggcctcgccg

SEQ ID NO: 62 - DNA, 21 bp, Artificial Sequence
  Mycobacterium intracellulare 3 capture probe
gcctcgccgcccaagacagtg

SEQ ID NO: 63 - DNA, 21 bp, Artificial Sequence
  Mycobacterium leprae capture probe
gatttcggcgtccatcggtgg

SEQ ID NO: 64 - DNA, 21 bp, Artificial Sequence
  Mycobacterium kansasi 1 capture probe
gatcgtcggcagtggtgacgg

SEQ ID NO: 65 - DNA, 17 bp, Artificial Sequence
  Mycobacterium kansasi 3 capture probe
tcgtcggcagtggtgac

SEQ ID NO: 66 - DNA, 27 bp, Artificial Sequence
  Mycobacterium kansasi 5 capture probe
atccgccgatcgtcggcagtggtgacg

SEQ ID NO: 67 - DNA, 21 bp, Artificial Sequence
  Mycobacterium malmoense capture probe
gacccacaacactggtcggcg

SEQ ID NO: 68 - DNA, 21 bp, Artificial Sequence
  Mycobacterium marinum capture probe
cggaggtgatggcgctggtcg

SEQ ID NO: 69 - DNA, 20 bp, Artificial Sequence
  Mycobacterium scrofulaceum  capture probe
cggcggcacggatcggcgtc

SEQ ID NO: 70 - DNA, 20 bp, Artificial Sequence
  Mycobacterium simiae capture probe
atcgctcctggtcgcgccta

SEQ ID NO: 71 - DNA, 21 bp, Artificial Sequence
  Mycobacterium szulgai capture probe
cccggcgcgaccagcagaacg

SEQ ID NO: 72 - DNA, 22 bp, Artificial Sequence
  Mycobacterium tuberculosis capture probe
gccgtccagtcgttaatgtcgc

SEQ ID NO: 73 - DNA, 22 bp, Artificial Sequence
  Mycobacterium xenopi capture probe
cggtagaagctgcgatgacacg

SEQ ID NO: 74 - DNA, 45 bp, Artificial Sequence
  Spacer Sequence
gaattcaaagttgctgagaatagttcaatggaaggaagcgtcttc

SEQ ID NO: 75 - DNA, 24 bp, Artificial Sequence
  consensus primer DPSCONS2 sense
gggctccagcagccaagaagagga

SEQ ID NO: 76 - DNA, 24 bp, Artificial Sequence
  consensus primer DPSMAGE1 sense
gggttccagcagccgtgaagagga

SEQ ID NO: 77 - DNA, 24 bp, Artificial Sequence
  consensus primer DPSMAG8 sense
gggttccagcagcaatgaagagga

SEQ ID NO: 78 - DNA, 24 bp, Artificial Sequence
  consensus primer DPSMAG12 sense
gggctccagcaacgaagaacagga

SEQ ID NO: 79 - DNA, 24 bp, Artificial Sequence
  consensus primer DPASCONB4 antisense
cggtactccaggtagttttcctgc

SEQ ID NO: 80 - DNA, 27 bp, Artificial Sequence
  capture probe Mage 1 DTAS01
acaaggactccaggatacaagaggtgc

SEQ ID NO: 81 - DNA, 27 bp, Artificial Sequence
  capture probe Mage 2 DTAS02
actcggactccaggtcgggaaacattc

SEQ ID NO: 82 - DNA, 27 bp, Artificial Sequence
  capture probe Mage 3 DTS0306
aagacagtatcttgggggatcccaaga

SEQ ID NO: 83 - DNA, 27 bp, Artificial Sequence
  capture probe Mage 4 DTAS04
tcggaacaaggactctgcgtcaggcga

SEQ ID NO: 84 - DNA, 27 bp, Artificial Sequence
  capture probe Mage 5 DTAS05
gctcggaacacagactctgggtcaggg

SEQ ID NO: 85 - DNA, 27 bp, Artificial Sequence
  capture probe Mage 6 DTS06
caagacaggcttcctgataatcatcct

SEQ ID NO: 86 - DNA, 27 bp, Artificial Sequence
  capture probe Mage 7 DTAS07
aggacgccaggtgagcggggtgtgtct

SEQ ID NO: 87 - DNA, 27 bp, Artificial Sequence
  capture probe Mage 8 DTAS08
gggactccaggtgagctgggtccgggg

SEQ ID NO: 88 - DNA, 27 bp, Artificial Sequence
  capture probe Mage 9 DTAS09
tgaactccagctgagctgggtcgaccg

SEQ ID NO: 89 - DNA, 27 bp, Artificial Sequence
  capture probe Mage 10 DTAS10
tgggtaaagactcactgtctggcagga

SEQ ID NO: 90 - DNA, 27 bp, Artificial Sequence
  capture probe Mage 11 DTAS11
gaaaaggactcagggtctatcaggtca

SEQ ID NO: 91 - DNA, 27 bp, Artificial Sequence
  capture probe Mage 12 DTAS12
gtgctacttggaagctcgtctccaggt

SEQ ID NO: 92 - DNA, 22 bp, Artificial Sequence
  consensus primer CONSENSUS2-3-4 sense
tgcagacmaccaccaactactt

SEQ ID NO: 93 - DNA, 22 bp, Artificial Sequence
  consensus primer CONSENSUS1-5 sense
tgmggkccaagatgaccaacwt

SEQ ID NO: 94 - DNA, 22 bp, Artificial Sequence
  consensus primer CONSENSUS1-5 antisense
tcatgrcrcasaggttcaggat

SEQ ID NO: 95 - DNA, 27 bp, Artificial Sequence
  capture probe DRD1
ctggcttttggcctttgggtccctttt

SEQ ID NO: 96 - DNA, 27 bp, Artificial Sequence
  capture probe DRD2
tgattggaaattcagcaggattcactg

SEQ ID NO: 97 - DNA, 27 bp, Artificial Sequence
  capture probe DRD3
gagtctggaatttcagccgcatttgct

SEQ ID NO: 98 - DNA, 27 bp, Artificial Sequence
  capture probe DRD4
cgtctggctgctgagcccccgcctctg

SEQ ID NO: 99 - DNA, 27 bp, Artificial Sequence
  capture probe DRD5
ctgggtactggccctttgggacattct

SEQ ID NO: 100 - DNA, 18 bp, Artificial Sequence
  primer H1sense
ctccgtccagcaacccct

SEQ ID NO: 101 - DNA, 19 bp, Artificial Sequence
  primer H2sense
ctgtgctggtcaccccagt

SEQ ID NO: 102 - DNA, 21 bp, Artificial Sequence
  primer H3sense
actcatcagctatgaccgatt

SEQ ID NO: 103 - DNA, 20 bp, Artificial Sequence
  primer H1antisense
accttccttggtatcgtctg

SEQ ID NO: 104 - DNA, 21 bp, Artificial Sequence
  primer H2 antisense
gaaaccagcagatgatgaacg

SEQ ID NO: 105 - DNA, 19 bp, Artificial Sequence
  primer H3 antisense
gcatctggtgggggttctg

SEQ ID NO: 106 - DNA, 18 bp, Artificial Sequence
  capture probe H1
ccccaggatggtagcgga

SEQ ID NO: 107 - DNA, 22 bp, Artificial Sequence
  capture probe H2
aggatagggtgatagaaataac

SEQ ID NO: 108 - DNA, 18 bp, Artificial Sequence
  capture probe H3
tctcgtgtccccctgctg

SEQ ID NO: 109 - DNA, 20 bp, Artificial Sequence
  general consensus primer sequence for subtypes 1A, 1B, 1C, 1D, 1E, 2A, 2B, 2C, 4, 6, and 7 sense
atchtgcacctstgbgbcat

SEQ ID NO: 110 - DNA, 20 bp, Artificial Sequence
  consensus primer for subtype 1A sense
atcctgcacctgtgcgccat

SEQ ID NO: 111 - DNA, 20 bp, Artificial Sequence
  consensus primer for subtype 1B sense
atcatgcatctctgtgtcat

SEQ ID NO: 112 - DNA, 20 bp, Artificial Sequence
  consensus primer for subtype 1C sense
atcatgcacctctgcgccat

SEQ ID NO: 113 - DNA, 20 bp, Artificial Sequence
  consensus primer for subtype 1D sense
atcctgcatctctgtgtcat

SEQ ID NO: 114 - DNA, 20 bp, Artificial Sequence
  consensus primer for subtype 1E sense
atcttgcacctgtcggctat

SEQ ID NO: 115 - DNA, 20 bp, Artificial Sequence
  consensus primer for subtype 2A sense
atcatgcacctctgcgccat

SEQ ID NO: 116 - DNA, 20 bp, Artificial Sequence
  consensus primer for subtype 2B sense
atcatgcatctctgtgccat

SEQ ID NO: 117 - DNA, 20 bp, Artificial Sequence
  consensus primer for subtype 2C sense
atcatgcacctctgcgccat

SEQ ID NO: 118 - DNA, 20 bp, Artificial Sequence
  consensus primer for subtype 4 sense
atttttcacctctgctgcat

SEQ ID NO: 119 - DNA, 20 bp, Artificial Sequence
  consensus primer for subtype 6 sense
atcctcaacctctgcttcat

SEQ ID NO: 120 - DNA, 20 bp, Artificial Sequence
  consensus primer for subtype 7 sense
atcatgaccctgtgcgtgat

SEQ ID NO: 121 - DNA, 20 bp, Artificial Sequence
  general consensus primer for 4, 6
atcytycacctctgcykcat

SEQ ID NO: 122 - DNA, 20 bp, Artificial Sequence
  consensus primer for 4
atttttcacctctgctgcat

SEQ ID NO: 123 - DNA, 20 bp, Artificial Sequence
  consensus primer for 6
atttttcacctctgctgcat

SEQ ID NO: 124 - DNA, 20 bp, Artificial Sequence
  general consensus primer for 5A, 5B
atctggaaygtgrcagccat

SEQ ID NO: 125 - DNA, 20 bp, Artificial Sequence
  consensus primer for 5A
atctggaatgtgacagcaat

SEQ ID NO: 126 - DNA, 20 bp, Artificial Sequence
  consensus primer for 5B
atctggaacgtggcggccat

SEQ ID NO: 127 - DNA, 20 bp, Artificial Sequence
  consensus primer for Specific 7
atcatgaccctgtgcgtgat

SEQ ID NO: 128 - DNA, 19 bp, Artificial Sequence
  consensus primer for Specific 3B
cttccggaacgattagaaa

SEQ ID NO: 129 - DNA, 17 bp, Artificial Sequence
  general consensus primer for Consensus subtypes 1A, 1B, 1C, 1D, 1E, 2A, 2B, 2C, 4, 7
  misc_feature (1)...(17): n = A,T,C or G
  misc_feature 6: n = A,T,C or G
  misc_feature 6: n = A,T,C or G
ttgghngcyttcygbtc

SEQ ID NO: 130 - DNA, 17 bp, Artificial Sequence
  consensus primer Consensus subtype 1A antisense
ttcaccgtcttcctttc

SEQ ID NO: 131 - DNA, 17 bp, Artificial Sequence
  consensus primer for Consensus subtype 1B antisense
ttggtggctttgcgctc

SEQ ID NO: 132 - DNA, 17 bp, Artificial Sequence
  consensus primer for Consensus subtype 1C antisense
ttggaagctttcttttc

SEQ ID NO: 133 - DNA, 17 bp, Artificial Sequence
  consensus primer for Consensus subtype 1D antisense
ttagtggctttcctttc

SEQ ID NO: 134 - DNA, 17 bp, Artificial Sequence
  consensus primer for Consensus subtype 1E antisense
gtggctgctttgcgttc

SEQ ID NO: 135 - DNA, 17 bp, Artificial Sequence
  consensus primer for Consensus subtype 2A antisense
ttgcacgccttttgctc

SEQ ID NO: 136 - DNA, 17 bp, Artificial Sequence
  consensus primer for Consensus subtype 2B antisense
tttgaggctctctgttc

SEQ ID NO: 137 - DNA, 17 bp, Artificial Sequence
  consensus primer for Consensus subtype 2C antisense
ttggaagctttcttttc

SEQ ID NO: 138 - DNA, 17 bp, Artificial Sequence
  consensus primer for Consensus subtype 4 antisense
ttggctgctttccggtc

SEQ ID NO: 139 - DNA, 17 bp, Artificial Sequence
  consensus primer for Consensus subtype 7 antisense
gtggctgctttctgttc

SEQ ID NO: 140 - DNA, 17 bp, Artificial Sequence
  consensus primer for Consensus Specific 1A antisense
ttcaccgtcttcctttc

SEQ ID NO: 141 - DNA, 17 bp, Artificial Sequence
  consensus primer for Consensus Specific 4 antisense
tcttggctgctttggtc

SEQ ID NO: 142 - DNA, 18 bp, Artificial Sequence
  consensus primer for Consensus Specific 6 antisense
ataaagagcgggtagatg

SEQ ID NO: 143 - DNA, 16 bp, Artificial Sequence
  consensus primer Consensus 5A,5B antisense
ccttctgctccctcca

SEQ ID NO: 144 - DNA, 16 bp, Artificial Sequence
  consensus primer for Consensus 5A antisense
ccttctgttccctcca

SEQ ID NO: 145 - DNA, 16 bp, Artificial Sequence
  consensus primer for Consensus 5B antisense
ccttctgctcccgcca

SEQ ID NO: 146 - DNA, 16 bp, Artificial Sequence
  consensus primer for Specific 3B antisense
accggggactctgtgt

SEQ ID NO: 147 - DNA, 21 bp, Artificial Sequence
  capture probe HTR1C
ctatgctcaataggattacgt

SEQ ID NO: 148 - DNA, 19 bp, Artificial Sequence
  capture probe HTR2A
gtggtgaatggggttctgg

SEQ ID NO: 149 - DNA, 21 bp, Artificial Sequence
  capture probe HTR2B
tggcctgaattggctttttga

SEQ ID NO: 150 - DNA, 22 bp, Artificial Sequence
  capture probe HTR2C/1C
ttattcacgaacactttgcttt

SEQ ID NO: 151 - DNA, 20 bp, Artificial Sequence
  capture probe HTR1B
aatagtccaccgcatcagtg

SEQ ID NO: 152 - DNA, 19 bp, Artificial Sequence
  capture probe HTR1D
gtactccagggcatcggtg

SEQ ID NO: 153 - DNA, 20 bp, Artificial Sequence
  capture probe HTR1A
catagtctatagggtcggtg

SEQ ID NO: 154 - DNA, 20 bp, Artificial Sequence
  capture probe HTR1E
atactcgactgcgtctgtga

SEQ ID NO: 155 - DNA, 19 bp, Artificial Sequence
  capture probe HTR7
gtacgtgaggggtctcgtg

SEQ ID NO: 156 - DNA, 19 bp, Artificial Sequence
  capture probe HTR5A
ggcgcgttattgaccagta

SEQ ID NO: 157 - DNA, 18 bp, Artificial Sequence
  capture probe HTR5B
ggcgcgtgatagtccagt

SEQ ID NO: 158 - DNA, 21 bp, Artificial Sequence
  capture probe HTR3B
gatatcaaaggggaaagcgta

SEQ ID NO: 159 - DNA, 20 bp, Artificial Sequence
  capture probe HTR4
aaaccaaaggttgacagcag

SEQ ID NO: 160 - DNA, 18 bp, Artificial Sequence
  capture probe HTR6
gtagcgcagcggcgagag

SEQ ID NO: 161 - DNA, 20 bp, Artificial Sequence
  consensus primer IPSCONA sense
gacagcgacgccgcgagcca

SEQ ID NO: 162 - DNA, 22 bp, Artificial Sequence
  consensus primer IPASCONA antisense
cgtgtcctgggtctggtcctcc

SEQ ID NO: 163 - DNA, 27 bp, Artificial Sequence
  capture probe HLA-A1 ITSA01
ggagggccggtgcgtggacgggctccg

SEQ ID NO: 164 - DNA, 27 bp, Artificial Sequence
  capture probe HLA-A2 ITASA02
tctccccgtcccaatactccggaccct

SEQ ID NO: 165 - DNA, 27 bp, Artificial Sequence
  capture probe HLA-A3 ITASA03A
ctgggccttcacattccgtgtctcctg

SEQ ID NO: 166 - DNA, 27 bp, Artificial Sequence
  capture probe HLA-A3 ITSA03B
agcgcaagtgggaggcggcccatgagg

SEQ ID NO: 167 - DNA, 27 bp, Artificial Sequence
  capture probe HLA-A11 ITSA11A
gcccatgcggcggagcagcagagagcc

SEQ ID NO: 168 - DNA, 27 bp, Artificial Sequence
  capture probe HLA-A11 ITSA11B
cctggagggccggtgcgtggagtggct

SEQ ID NO: 169 - DNA, 27 bp, Artificial Sequence
  capture probe HLA-A23 ITSA23A
gcccgtgtggcggagcagttgagagcc

SEQ ID NO: 170 - DNA, 27 bp, Artificial Sequence
  capture probe HLA-A23 ITASA23B
ccttcactttccctgtctcctcgtccc

SEQ ID NO: 171 - DNA, 27 bp, Artificial Sequence
  capture probe HLA-A24 ITSA24A
gcccatgtggcggagcagcagagagcc

SEQ ID NO: 172 - DNA, 27 bp, Artificial Sequence
  capture probe HLA-A24 ITASA24B
tagcggagcgcgatccgcaggttctct

SEQ ID NO: 173 - DNA, 27 bp, Artificial Sequence
  capture probe HLA-A25 ITASA25A
tagcggagcgcgatccgcaggctctct

SEQ ID NO: 174 - DNA, 27 bp, Artificial Sequence
  capture probe HLA-A25 ITASA25B
tcacattccgtgtgttccggtcccaat

SEQ ID NO: 175 - DNA, 27 bp, Artificial Sequence
  capture probe HLA-A26 ITASA26
gggtccccaggttcgctcggtcagtct

SEQ ID NO: 176 - DNA, 27 bp, Artificial Sequence
  capture probe HLA-A29 ITASA29
tcacattccgtgtctgcaggtcccaat

SEQ ID NO: 177 - DNA, 27 bp, Artificial Sequence
  capture probe HLA-A30 ITASA30
cgtaggcgtgctgttcatacccgcgga

SEQ ID NO: 178 - DNA, 27 bp, Artificial Sequence
  capture probe HLA-A31 ITASA31
cccaatactcaggcctctcctgctcta

SEQ ID NO: 179 - DNA, 27 bp, Artificial Sequence
  capture probe HLA-A33 ITSA33
cgcacggacccccccaggacgcatatg

SEQ ID NO: 180 - DNA, 27 bp, Artificial Sequence
  capture probe HLA-A68 ITSA68A
ggcggcccatgtggcggagcagtggag

SEQ ID NO: 181 - DNA, 27 bp, Artificial Sequence
  capture probe HLA-A68 ITASA68B
gtcgtaggcgtcctgccggtacccgcg

SEQ ID NO: 182 - DNA, 27 bp, Artificial Sequence
  capture probe HLA-A69 ITASA69
atcctctggacggtgtgagaaccggcc

SEQ ID NO: 183 - DNA, 15 bp, Artificial Sequence
  sense primer for cytochrome P450
gccagagcctgagga

SEQ ID NO: 184 - DNA, 20 bp, Artificial Sequence
  primer consensus a3, a23, a1, a2  antisense
tcaaaagaaattaacagaga

SEQ ID NO: 185 - DNA, 19 bp, Artificial Sequence
  primer Specific a9  antisense
acaatgaaggtaacatagg

SEQ ID NO: 186 - DNA, 18 bp, Artificial Sequence
  primer Specific a18  antisense
actgatggaactaactgg

SEQ ID NO: 187 - DNA, 23 bp, Artificial Sequence
  capture probe 3a1
tgttttgattcggtacatctttg

SEQ ID NO: 188 - DNA, 21 bp, Artificial Sequence
  capture probe 3a3
ttgatttggtacatctttgct

SEQ ID NO: 189 - DNA, 20 bp, Artificial Sequence
  capture probe 3A9
actcctgggggttttgggtg

SEQ ID NO: 190 - DNA, 24 bp, Artificial Sequence
  capture probe 3A18
attactgagtattcagaaattcac

SEQ ID NO: 191 - DNA, 25 bp, Artificial Sequence
  capture probe 3A2
ggttaaagatttggtacatttatgg

SEQ ID NO: 192 - DNA, 22 bp, Artificial Sequence
  consensus primer OPP35S1 (P-35S) Forward
cgtcttcaaagcaagtggattg

SEQ ID NO: 193 - DNA, 24 bp, Artificial Sequence
  consensus primer OPT352 (T-35S) Reverse
gaaaccctaattcccttatcaggg

SEQ ID NO: 194 - DNA, 25 bp, Artificial Sequence
  consensus primer OPTE91 (T-E91) Forward
tcatggatttgtagttgagtatgaa

SEQ ID NO: 195 - DNA, 27 bp, Artificial Sequence
  consensus primer OPTnos2 (T-nos) Reverse
atcttaagaaactttattgccaaatgt

SEQ ID NO: 196 - DNA, 21 bp, Artificial Sequence
  consensus primer OPEPS3 (EPSPS) Forward
gctgtagttgttggctgtggt

SEQ ID NO: 197 - DNA, 21 bp, Artificial Sequence
  consensus primer OPTE92 (T-E9) Reverse
ctgatgcattgaacttgacga

SEQ ID NO: 198 - DNA, 25 bp, Artificial Sequence
  consensus primer OPLB1 (octopine Left Border) Forward
atcagcaatgagtatgatggtcaat

SEQ ID NO: 199 - DNA, 20 bp, Artificial Sequence
  consensus primer OPEPS4 (EPSPS) Reverse
gcgacatcaggcatcttgtt

SEQ ID NO: 200 - DNA, 22 bp, Artificial Sequence
  consensus primer OPLB3 (nopaline Left Border) Forward
acaaattgacgcttagacaact

SEQ ID NO: 201 - DNA, 21 bp, Artificial Sequence
  consensus primer OPRB2 (octopine Right Border) Reverse
tgccagtcagcatcatcacac

SEQ ID NO: 202 - DNA, 21 bp, Artificial Sequence
  consensus primer OPRB4 (nopaliine Right Border) Reverse
taagggagtcacgttatgacc

SEQ ID NO: 203 - DNA, 30 bp, Artificial Sequence
  capture probe OT1 pat (T25, Bt11)
tggtggatggcatgatgttggtttttggca

SEQ ID NO: 204 - DNA, 30 bp, Artificial Sequence
  capture probe OT2 CryIAb (Bt11)
gcacgaagctctgcaatcgcacaaacccgt

SEQ ID NO: 205 - DNA, 30 bp, Artificial Sequence
  capture probe OT3 P-PCK (Bt176)
tgggggtagctgtagtcggactcggactgg

SEQ ID NO: 206 - DNA, 30 bp, Artificial Sequence
  capture probe OT4 CP4EPSPS/Tnos
agcccctagctagggggtggccaggaagta

SEQ ID NO: 207 - DNA, 20 bp, Artificial Sequence
  consensus primer Pgyr1
  misc_feature (1)...(20): n = A,T,C or G
  misc_feature 3, 6: n = A,T,C or G
  misc_feature 3, 6: n = A,T,C or G
gangtnatsggtaaatayca

SEQ ID NO: 208 - DNA, 17 bp, Artificial Sequence
  consensus primer Pgyr2
  misc_feature (1)...(17): n = A,T,C or G
  misc_feature 3: n = A,T,C or G
  misc_feature 3: n = A,T,C or G
cgnryytcvgtrtaacg

SEQ ID NO: 209 - DNA, 90 bp, Artificial Sequence
  spacer sequence
ataaaaaagtgggtcttagaaataaatttcgaagtgcaataattattattcacaacatttcgatttttgcaactacttcagttcactcca

SEQ ID NO: 210 - DNA, 34 bp, Staphylococcus genus
gactcwtcaatttatgawgchatggtahgaaygg

SEQ ID NO: 211 - DNA, 30 bp, Enterococcus genus
gacagtgcgatytaygartcaatggtrcgg

SEQ ID NO: 212 - DNA, 29 bp, Streptococcus genus
tggttcgtatggctcaatggtggagytay

SEQ ID NO: 213 - DNA, 28 bp, Staphylococcus aureus
ctcaagatttcagttatcgttatccgct

SEQ ID NO: 214 - DNA, 28 bp, Staphylococcus epidermidis
cccaagactttagttatcgttatccact

SEQ ID NO: 215 - DNA, 27 bp, Staphylococcus hominis
cacaaacctttagctatcgttatcctc

SEQ ID NO: 216 - DNA, 27 bp, Enterococcus faecium
acagccattcagctaccgttatatgct

SEQ ID NO: 217 - DNA, 29 bp, Enterococcus faecalis
aaccttttagttatcgggctatgttagtt

SEQ ID NO: 218 - DNA, 25 bp, Streptococcus pneumoniae
gatggagatagtgctgccgctcaac

SEQ ID NO: 219 - DNA, 26 bp, Streptococcus epyogenes
cttgttgatgggcatggcaattttgg

SEQ ID NO: 220 - DNA, 28 bp, Haemophilus influenzae
ttctcacttcgctatatgttggttgatg

SEQ ID NO: 221 - DNA, 27 bp, Artificial Sequence
  5′ aminated primer
gaattcaaagttgctgagaatagttca

SEQ ID NO: 222 - DNA, 16 bp, Artificial Sequence
  viral consensus primer
  misc_difference (1)...(1): N is preferably A or G
  misc_difference (2)...(2): N is preferably C, A or T
  misc_difference (3)...(3): N is preferably G, C or T
  misc_difference (13)...(13): N is preferably T or A
  misc_difference (14)...(14): N is preferably T, A or C
  misc_difference (15)...(15): N is preferably C or T
  misc_difference (16)...(16): N is preferably G, A or C
nnngccgccgtgnnnn

SEQ ID NO: 223 - DNA, 16 bp, Artificial Sequence
  viral consensus primer
  misc_difference (2)...(2): N is preferably T, G or C
  misc_difference (3)...(3): N is preferably G, T or A
  misc_difference (13)...(13): N is preferably G or A
  misc_difference (14)...(14): N is preferably T or C
  misc_difference (15)...(15): N is preferably G or C
  misc_difference (16)...(16): N is preferably G or T
gnngttgtttttnnnn

SEQ ID NO: 224 - DNA, 27 bp, Artificial Sequence
  Adenovirus capture nucleotide sequence
aactcttctcgctggcactcaagagtg

SEQ ID NO: 225 - DNA, 27 bp, Artificial Sequence
  Herpes virus 1 capture nucleotide sequence
gtggaagtcctgatacccatcctacac

SEQ ID NO: 226 - DNA, 27 bp, Artificial Sequence
  Herpes virus 5 capture nucleotide sequence
aaaagcgtgtgatctgaccgaggcgaa

SEQ ID NO: 227 - DNA, 27 bp, Artificial Sequence
  Herpes virus 4 capture nucleotide sequence
aggtccttgaggaagaagtgttccagg

SEQ ID NO: 228 - DNA, 21 bp, Artificial Sequence
  consensus primer Meat1
tcctcccatgaggagaaatat

SEQ ID NO: 229 - DNA, 21 bp, Artificial Sequence
  consensus primer Meat2
agcgaagaatcgggtaagggt

SEQ ID NO: 230 - DNA, 39 bp, Artificial Sequence
  Chicken capture nucleotide sequence
ccttaacgactcttatccaaacactatgccaccggggag

SEQ ID NO: 231 - DNA, 40 bp, Artificial Sequence
  Duck capture nucleotide sequence
ccctaacgactcttatccaaacactactgccatcggggag

SEQ ID NO: 232 - DNA, 17 bp, Artificial Sequence
  Ostrich capture nucleotide sequence
ccttaacgaactctaag

SEQ ID NO: 233 - DNA, 25 bp, Artificial Sequence
  Pig capture nucleotide sequence
aaagaggagtagaatcacgattaag

SEQ ID NO: 234 - DNA, 40 bp, Artificial Sequence
  Quail capture nucleotide sequence
ccatgtcgactcttatccaaacactactgccatcgtggag

SEQ ID NO: 235 - DNA, 40 bp, Artificial Sequence
  Rabbit capture nucleotide sequence
ccctaacgactatcctccaatcactaatgccaacgagggg

SEQ ID NO: 236 - DNA, 39 bp, Artificial Sequence
  Turkey capture nucleotide sequence
ccctaacgactcttatccaaacactactgccatcgggag

SEQ ID NO: 237 - DNA, 40 bp, Artificial Sequence
  Wildpig capture nucleotide sequence
ccctatcgactatcttctaaacactactggcatcgaggag

SEQ ID NO: 238 - DNA, 39 bp, Artificial Sequence
  Cow capture nucleotide sequence
cctaacgactattctccaaccactactgacaacgaggag

SEQ ID NO: 239 - DNA, 96 bp, Artificial Sequence
  consensus capture nucleotide sequence for cytochrome b
attctgaggggcaccgtcatcacaaacctatttcagcaatcccctacatggcaaaccctagtagaatgagcctgagggggattttcagtgacaacc

SEQ ID NO: 240 - DNA, 23 bp, Artificial Sequence
  consensus primer Cow1
aagacataatatgtatatagtac

SEQ ID NO: 241 - DNA, 23 bp, Artificial Sequence
  consensus primer Cow2
gaaaaatttaaataagtatctag

SEQ ID NO: 242 - DNA, 15 bp, Artificial Sequence
  BrownSwiss capture nucleotide sequence
gcggcatgataatta

SEQ ID NO: 243 - DNA, 15 bp, Artificial Sequence
  Jersey capture nucleotide sequence
cgctattcaatgaat

SEQ ID NO: 244 - DNA, 15 bp, Artificial Sequence
  Ayrshire capture nucleotide sequence
gctcaccataactgt

SEQ ID NO: 245 - DNA, 15 bp, Artificial Sequence
  Hereford capture nucleotide sequence
atctgatggtaagga

SEQ ID NO: 246 - DNA, 15 bp, Artificial Sequence
  Simmental capture nucleotide sequence
ataagcctggacatt

SEQ ID NO: 247 - DNA, 15 bp, Artificial Sequence
  Piemontaise capture nucleotide sequence
ataagcatggacatt

SEQ ID NO: 248 - DNA, 15 bp, Artificial Sequence
  Canadienne capture nucleotide sequence
tcactcggcatgata

SEQ ID NO: 249 - DNA, 15 bp, Artificial Sequence
  RedAngus capture nucleotide sequence
aatggtaggggatat

SEQ ID NO: 250 - DNA, 15 bp, Artificial Sequence
  Limousine capture nucleotide sequence
atggactcatggcta

SEQ ID NO: 251 - DNA, 15 bp, Artificial Sequence
  AberdeenAngus capture nucleotide sequence
tattcaatgaacttt

SEQ ID NO: 252 - DNA, 15 bp, Artificial Sequence
  Butana capture nucleotide sequence
gcatggggtatataa

SEQ ID NO: 253 - DNA, 16 bp, Artificial Sequence
  Charolais capture nucleotide sequence
ataagcgtggacatta

SEQ ID NO: 254 - DNA, 15 bp, Artificial Sequence
  Fresian capture nucleotide sequence
ccttaaatacctacc

SEQ ID NO: 255 - DNA, 15 bp, Artificial Sequence
  Kenana capture nucleotide sequence
tgctatagaagtcat

SEQ ID NO: 256 - DNA, 15 bp, Artificial Sequence
  N ′Dama capture nucleotide sequence
tgttatagaagtcat

SEQ ID NO: 257 - DNA, 20 bp, Artificial Sequence
  consensus primer PPss3
  misc_feature (1)...(20): n = A,T,C or G
  misc_feature 15: n = A,T,C or G
  misc_feature 15: n = A,T,C or G
ggtttggagarrggntgggg

SEQ ID NO: 258 - DNA, 20 bp, Artificial Sequence
  consensus primer PPss4
tccaadatgtavacaacctg

SEQ ID NO: 259 - DNA, 24 bp, Artificial Sequence
  TPss1 (potato) capture nucleotide sequence
gaagcatgcataccatctctagca

SEQ ID NO: 260 - DNA, 24 bp, Artificial Sequence
  TPss3 (tomato) capture nucleotide sequence
ggagcatgcagatcatctctagaa

SEQ ID NO: 261 - DNA, 24 bp, Artificial Sequence
  TPss7 (oryza) capture nucleotide sequence
gaagcaagtggatggtgtcaagca

SEQ ID NO: 262 - DNA, 24 bp, Artificial Sequence
  TPss8 (zea) capture nucleotide sequence
agaggaggtggatagtctcctgtg

SEQ ID NO: 263 - DNA, 24 bp, Artificial Sequence
  TPss9 (soja) capture nucleotide sequence
agagaagttgaattgactcaagga

SEQ ID NO: 264 - DNA, 24 bp, Artificial Sequence
  TPss11 (wheat) capture nucleotide sequence
agagaaggtggatagtctcgctcg

SEQ ID NO: 265 - DNA, 24 bp, Artificial Sequence
  TPss12 (bareley) capture nucleotide sequence
agagaaggtggatagtctcgctcg

SEQ ID NO: 266 - DNA, 24 bp, Artificial Sequence
  TPss13 (bean) capture nucleotide sequence
atagaagctgaatggactcgagca

SEQ ID NO: 267 - DNA, 24 bp, Artificial Sequence
  TPss14 (carrot) capture nucleotide sequence
gaagcatgtgaaacatctcagtaa

SEQ ID NO: 268 - DNA, 21 bp, Artificial Sequence
  Fish1 consensus primer
actatthctagccatvcayta

SEQ ID NO: 269 - DNA, 23 bp, Artificial Sequence
  Fish2 consensus primer
aggtaggagccataaagacctcg

SEQ ID NO: 270 - DNA, 28 bp, Artificial Sequence
  G. morhua capture nucleotide sequence
aaggcttaatcagtcggcatcaaatgta

SEQ ID NO: 271 - DNA, 28 bp, Artificial Sequence
  G. macrocephalus capture nucleotide sequence
aaggcttactcagttggcattaaatgta

SEQ ID NO: 272 - DNA, 28 bp, Artificial Sequence
  P. flesus capture nucleotide sequence
gaagcctactcagttggcatcaactgca

SEQ ID NO: 273 - DNA, 28 bp, Artificial Sequence
  M. merluccius capture nucleotide sequence
aacgcctaatcagtaggcattaaatgca

SEQ ID NO: 274 - DNA, 28 bp, Artificial Sequence
  O. mykiss capture nucleotide sequence
aaagcttactcagtcggcattgattgta

SEQ ID NO: 275 - DNA, 28 bp, Artificial Sequence
  P. platessa capture nucleotide sequence
gaagcctattcagtcggcatcaactgca

SEQ ID NO: 276 - DNA, 28 bp, Artificial Sequence
  P. virens capture nucleotide sequence
aaagcttaattagtcggcattaaatgta

SEQ ID NO: 277 - DNA, 29 bp, Artificial Sequence
  S. salar capture nucleotide sequence
caatgcctactcagtcggtatcgattgta

SEQ ID NO: 278 - DNA, 28 bp, Artificial Sequence
  S. pilchardus capture nucleotide sequence
gaagcttagtcagtaggcatcaaatgca

SEQ ID NO: 279 - DNA, 28 bp, Artificial Sequence
  A. thazard capture nucleotide sequence
aaagcctattcagttggcttcaaatgta

SEQ ID NO: 280 - DNA, 28 bp, Artificial Sequence
  T. alalunga capture nucleotide sequence
aaagcctactcagtaggcttcaaatgta

SEQ ID NO: 281 - DNA, 29 bp, Artificial Sequence
  T. obesus capture nucleotide sequence
aaagcctactcagttggctttaactgtta

SEQ ID NO: 282 - DNA, 28 bp, Artificial Sequence
  R. hippoglossoides capture nucleotide sequence
gaagcctattcagtcggcatcaactgca

SEQ ID NO: 283 - DNA, 28 bp, Artificial Sequence
  S. trutta capture nucleotide sequence
aaagcctactcagtcggcatcgattgca

SEQ ID NO: 284 - DNA, 28 bp, Artificial Sequence
  S. sarda capture nucleotide sequence
aaagcctaatcagtcggctttaattgca

SEQ ID NO: 285 - DNA, 28 bp, Artificial Sequence
  T. thynnus capture nucleotide sequence
aaggcctattcagttggcttcaactgta

SEQ ID NO: 286 - DNA, 28 bp, Artificial Sequence
  S. scrombrus capture nucleotide sequence
aacgcctactcagtaggcttcaaatgca

SEQ ID NO: 287 - DNA, 40 bp, Artificial Sequence
  Salmonidae family capture nucleotide sequence
aaacattcacgctaacggagcatctttcttctttatctgt

SEQ ID NO: 288 - DNA, 40 bp, Artificial Sequence
  Pleuronectidae family capture nucleotide sequence
aagcattcatgccaacggcgcatcattctttttcatttgc

SEQ ID NO: 289 - DNA, 40 bp, Artificial Sequence
  Pleuronectidae family capture nucleotide sequence
gaatatacatgctaatggtgcctctttcttttttatttgt

SEQ ID NO: 290 - DNA, 41 bp, Artificial Sequence
  Scrombridae family capture nucleotide sequence
aaacctccacgcaaacggagcctctttctttctttatctgc

SEQ ID NO: 291 - DNA, 15 bp, Artificial Sequence
  Thunnus genus capture nucleotide sequence
attccacatcggccg

SEQ ID NO: 292 - DNA, 57 bp, Artificial Sequence
  Fish consensus capture nucleotide sequence
atccgaaacatccacgcaacgggcatctttcttctttatctgtatctacttacacat

SEQ ID NO: 293 - DNA, 24 bp, Artificial Sequence
  P450-1 consensus primer
tccgcaacttgggcctgggcaaga

SEQ ID NO: 294 - DNA, 23 bp, Artificial Sequence
  P450-2 consensus primer
ccttctccatctctgccaggaag

SEQ ID NO: 295 - DNA, 17 bp, Artificial Sequence
  Human CYP2D6 wild-type capture nucleotide sequence
gaaaggggcgtcctggg

SEQ ID NO: 296 - DNA, 17 bp, Artificial Sequence
  Human CYP2D6 capture nucleotide sequence, point mutation
gaaaggggcgtcttggg

SEQ ID NO: 297 - DNA, 17 bp, Artificial Sequence
  Human CYP2D6 capture nucleotide sequence, wild type
gctaactgagcacagga

SEQ ID NO: 298 - DNA, 16 bp, Artificial Sequence
  Human CYP2D6 capture nucleotide sequence (deletion)
gctaactgagcacgga

SEQ ID NO: 299 - DNA, 16 bp, Artificial Sequence
  Human CYP2D6 capture nucleotide sequence, wild type
ctcggtcaccccctgc

SEQ ID NO: 300 - DNA, 15 bp, Artificial Sequence
  Human CYP2D6 capture nucleotide sequence (deletion)
ctcggtcacccctgc

SEQ ID NO: 301 - DNA, 16 bp, Artificial Sequence
  Human CYP2C19 capture nucleotide sequence, wild type
aattatttcccaggaa

SEQ ID NO: 302 - DNA, 16 bp, Artificial Sequence
  Human CYP2C19 capture nucleotide sequence, point mutation
aattatttcccaggaa

SEQ ID NO: 303 - DNA, 16 bp, Artificial Sequence
  Human CYP2C19 capture nucleotide sequence, wild type
agcaccccctgaatcc

SEQ ID NO: 304 - DNA, 16 bp, Artificial Sequence
  Human CYP2C19 capture nucleotide sequence, point mutation
agcaccccctgaatcc

SEQ ID NO: 305 - DNA, 18 bp, Artificial Sequence
  sense consensus primer, S. saprophyticus
gaattrgttgaaatggaa

SEQ ID NO: 306 - DNA, 17 bp, Artificial Sequence
  antisense consensus primer, S. saprophyticus
gtagtacggaartagaa

SEQ ID NO: 307 - DNA, 16 bp, Artificial Sequence
  sense primer of double labelled probe (S. saprophyticus)
ggtgttgaaatgttcc

SEQ ID NO: 308 - DNA, 17 bp, Artificial Sequence
  Meningococcus capture probe
cgacctgctgtccagct

SEQ ID NO: 309 - DNA, 18 bp, Artificial Sequence
  Streptococcus capture probe
cttcaggacgtatcgacc

SEQ ID NO: 310 - DNA, 19 bp, Artificial Sequence
  Staphylococcus capture probe
ttattagactacgctgaag

SEQ ID NO: 311 - DNA, 18 bp, Artificial Sequence
  N. menengitidis serogroup A capture probe
tctatttccggtcgtggt

SEQ ID NO: 312 - DNA, 16 bp, Artificial Sequence
  N. menengitidis serogroup B capture probe
ccatttccggccgcgg

SEQ ID NO: 313 - DNA, 19 bp, Artificial Sequence
  H. influenzae capture probe
gagttagcaaaccacttag

SEQ ID NO: 314 - DNA, 17 bp, Artificial Sequence
  E. coli capture probe
aactggctggcttcctg

SEQ ID NO: 315 - DNA, 21 bp, Artificial Sequence
  S. pneumoniae capture probe
gtatcaaagaagaaactcaaa

SEQ ID NO: 316 - DNA, 21 bp, Artificial Sequence
  S. agalactiae capture probe
gtattaaagaagatatccaaa

SEQ ID NO: 317 - DNA, 20 bp, Artificial Sequence
  S. aureus capture probe
ggtttacatgacacatctaa

SEQ ID NO: 318 - DNA, 20 bp, Artificial Sequence
  S. epidermidis capture probe
gtatgcacgaaacttctaaa

SEQ ID NO: 319 - DNA, 20 bp, Artificial Sequence
  S. haemolyticus capture probe
gtatccatgacacttctaaa

SEQ ID NO: 320 - DNA, 21 bp, Artificial Sequence
  S. hominis capture probe
ggtatcaaagaaacttctaaa

SEQ ID NO: 321 - DNA, 20 bp, Artificial Sequence
  S. saprophyticus capture probe
atgcaagaagaatcaagcaa

SEQ ID NO: 322 - DNA, 42 bp, Artificial Sequence
  synthetic primer
tctatatgctccacagtatgcgaacaagcgtgtcgtgctcca

SEQ ID NO: 323 - DNA, 43 bp, Artificial Sequence
  synthetic primer
actatatgctccactctatgcctcgacatgatactccttccac

SEQ ID NO: 324 - DNA, 43 bp, Artificial Sequence
  synthetic primer
tctatatgctccacagtatgcgacaatggacacatgaattatg

SEQ ID NO: 325 - DNA, 43 bp, Artificial Sequence
  synthetic primer
actatatgctccactctatgcctgactctgtatgaactgttcg

SEQ ID NO: 326 - DNA, 44 bp, Artificial Sequence
  synthetic primer
tctatatgctccacagtatgcgatttcccattcgagtttctcac

SEQ ID NO: 327 - DNA, 42 bp, Artificial Sequence
  synthetic primer
actatatgctccactctatgcctaaccaatgccaccccactg

SEQ ID NO: 328 - DNA, 42 bp, Artificial Sequence
  synthetic primer
tctatatgctccacagtatgcgactggcgtaatagcgaagag

SEQ ID NO: 329 - DNA, 42 bp, Artificial Sequence
  synthetic primer
actatatgctccactctatgcctctaacgggtgcatcgtcta

SEQ ID NO: 330 - DNA, 41 bp, Artificial Sequence
  synthetic primer
tctatatgctccacagtatgcgacgctctgtacaagcgtgc

SEQ ID NO: 331 - DNA, 43 bp, Artificial Sequence
  synthetic primer
actatatgctccactctatgcctcaaagtgttgtgcttggacc

SEQ ID NO: 332 - DNA, 23 bp, Artificial Sequence
  synthetic primer
tctatatgctccacagtatgcga

SEQ ID NO: 333 - DNA, 23 bp, Artificial Sequence
  synthetic primer
actatatgctccactctatgcct

SEQ ID NO: 334 - DNA, 25 bp, Artificial Sequence
  capture probe
tcattgagtcgttccgccattgtcg

SEQ ID NO: 335 - DNA, 23 bp, Artificial Sequence
  capture probe
gaggacctaacagaactcgccgt

SEQ ID NO: 336 - DNA, 25 bp, Artificial Sequence
  capture probe
ttgtccctccacttcttctctgcta

SEQ ID NO: 337 - DNA, 28 bp, Artificial Sequence
  capture probe
gcaccgattatttatacttttagtccac

SEQ ID NO: 338 - DNA, 24 bp, Artificial Sequence
  capture probe
ttagacgggaaaacgagaggaagc

SEQ ID NO: 339 - DNA, 95 bp, Artificial Sequence
  synthetic spacer
ataaaaaagtgggtcttagaaataaatttcgaagtgcaataattattattcacaacatttcgatttttgcaactacttcagttcactccaaatta

SEQ ID NO: 340 - DNA, 42 bp, Artificial Sequence; identical to SEQ ID NO: 322
  synthetic primer

SEQ ID NO: 341 - DNA, 43 bp, Artificial Sequence; identical to SEQ ID NO: 323
  synthetic primer

SEQ ID NO: 342 - DNA, 43 bp, Artificial Sequence; identical to SEQ ID NO: 324
  synthetic primer

SEQ ID NO: 343 - DNA, 43 bp, Artificial Sequence; identical to SEQ ID NO: 325
  synthetic primer

SEQ ID NO: 344 - DNA, 44 bp, Artificial Sequence; identical to SEQ ID NO: 326
  synthetic primer

SEQ ID NO: 345 - DNA, 42 bp, Artificial Sequence; identical to SEQ ID NO: 327
  synthetic primer

SEQ ID NO: 346 - DNA, 42 bp, Artificial Sequence; identical to SEQ ID NO: 328
  synthetic primer

SEQ ID NO: 347 - DNA, 42 bp, Artificial Sequence; identical to SEQ ID NO: 329
  synthetic primer

SEQ ID NO: 348 - DNA, 45 bp, Artificial Sequence
  synthetic primer
tctatatgctccacagtatgcgacatattgaccatcatacttgct

SEQ ID NO: 349 - DNA, 45 bp, Artificial Sequence
  synthetic primer
actatatgctccactctatgcctttatacgaaggcaagaaaagca

SEQ ID NO: 350 - DNA, 50 bp, Artificial Sequence
  synthetic primer
tctatatgctccacagtatgcgaatgaatgacctcgagtaagcttgttaa

SEQ ID NO: 351 - DNA, 45 bp, Artificial Sequence
  synthetic primer
actatatgctccactctatgcctaagagataacaggatccactca

SEQ ID NO: 352 - DNA, 40 bp, Artificial Sequence
  synthetic primer
tctatatgctccacagtatgcgagcggaacccctatttgt

SEQ ID NO: 353 - DNA, 41 bp, Artificial Sequence
  synthetic primer
actatatgctccactctatgccttccaagaatccctccatg

SEQ ID NO: 354 - DNA, 43 bp, Artificial Sequence
  synthetic primer
tctatatgctccacagtatgcgacctttaggatttcagcatca

SEQ ID NO: 355 - DNA, 38 bp, Artificial Sequence
  synthetic primer
actatatgctccactctatgcctgacttgtcgccggga

SEQ ID NO: 356 - DNA, 44 bp, Artificial Sequence
  synthetic primer
tctatatgctccacagtatgcgatgtcaaaacacaatttacagc

SEQ ID NO: 357 - DNA, 41 bp, Artificial Sequence
  synthetic primer
actatatgctccactctatgccttcccttaattctccgctc

SEQ ID NO: 358 - DNA, 41 bp, Artificial Sequence
  synthetic primer
tctatatgctccacagtatgcgaggatctgggtggctctaa

SEQ ID NO: 359 - DNA, 42 bp, Artificial Sequence
  synthetic primer
actatatgctccactctatgcctaatgctgctaaatcctgag

SEQ ID NO: 360 - DNA, 41 bp, Artificial Sequence
  synthetic primer
tctatatgctccacagtatgcgataggatcggaaagcttgg

SEQ ID NO: 361 - DNA, 41 bp, Artificial Sequence
  synthetic primer
actatatgctccactctatgcctttacggcctaaatgctga

SEQ ID NO: 362 - DNA, 41 bp, Artificial Sequence
  synthetic primer
tctatatgctccacagtatgcgatagtcttcggccagaatg

SEQ ID NO: 363 - DNA, 41 bp, Artificial Sequence
  synthetic primer
actatatgctccactctatgcctctttgccaagatcaagcg

SEQ ID NO: 364 - DNA, 42 bp, Artificial Sequence
  synthetic primer
tctatatgctccacagtatgcgaggataagcaagtaaaagcg

SEQ ID NO: 365 - DNA, 43 bp, Artificial Sequence
  synthetic primer
actatatgctccactctatgcctcttaattctccgctcatgat

SEQ ID NO: 366 - DNA, 23 bp, Artificial Sequence
  synthetic primer
tctatatgctccacagtatgcga

SEQ ID NO: 367 - DNA, 23 bp, Artificial Sequence
  synthetic primer
actatatgctccactctatgcct

SEQ ID NO: 368 - DNA, 25 bp, Artificial Sequence
  capture probe
tcattgagtcgttccgccattgtcg

SEQ ID NO: 369 - DNA, 23 bp, Artificial Sequence
  capture probe
gaggacctaacagaactcgccgt

SEQ ID NO: 370 - DNA, 25 bp, Artificial Sequence
  capture probe
ttgtccctccacttcttctctgcta

SEQ ID NO: 371 - DNA, 28 bp, Artificial Sequence
  capture probe
gcaccgattatttatacttttagtccac

SEQ ID NO: 372 - DNA, 27 bp, Artificial Sequence
  capture probe
ttcccggacatgaagatcatggtcctt

SEQ ID NO: 373 - DNA, 23 bp, Artificial Sequence
  capture probe
gtaccacgcgacacacttccact

SEQ ID NO: 374 - DNA, 27 bp, Artificial Sequence
  capture probe
aaatacattcaaatatgtatccgctca

SEQ ID NO: 375 - DNA, 19 bp, Artificial Sequence
  capture probe
cgcaaccgcccgcaaatcc

SEQ ID NO: 376 - DNA, 25 bp, Artificial Sequence
  capture probe
agattgtcgtttcccgccttcagtt

SEQ ID NO: 377 - DNA, 20 bp, Artificial Sequence
  capture probe
aaggcgggaaacgacaatct

SEQ ID NO: 378 - DNA, 24 bp, Artificial Sequence
  capture probe
tgaacacccatccgaacaagtagg

SEQ ID NO: 379 - DNA, 21 bp, Artificial Sequence
  capture probe
taactcaaggccctcactccg

SEQ ID NO: 380 - DNA, 26 bp, Artificial Sequence
  capture probe
tttaaactgaaggcgggaaacgacaa

SEQ ID NO: 381 - DNA, 94 bp, Artificial Sequence
  synthetic spacer
ataaaaaagtgggtcttagaaataaatttcgaagtgcaataattattattcacaacatttcgatttttgcaactacttcagtcactccaaatta

SEQ ID NO: 382 - DNA, 44 bp, Artificial Sequence
  synthetic primer
tctatatgctccacagtatgcgagacggcctgaagccggtgcac

SEQ ID NO: 383 - DNA, 44 bp, Artificial Sequence
  synthetic primer
actatatgctccactctatgcctgcccacggcgataccgctgga

SEQ ID NO: 384 - DNA, 45 bp, Artificial Sequence
  synthetic primer
tctatatgctccacagtatgcgacctgaccatccgtcgccacaac

SEQ ID NO: 385 - DNA, 43 bp, Artificial Sequence
  synthetic primer
actatatgctccactctatgcctcgcagcaggatgaagacgcc

SEQ ID NO: 386 - DNA, 41 bp, Artificial Sequence
  synthetic primer
tctatatgctccacagtatgcgagctctggctctggctgct

SEQ ID NO: 387 - DNA, 41 bp, Artificial Sequence
  synthetic primer
actatatgctccactctatgcctagggcacgctcgttagcc

SEQ ID NO: 388 - DNA, 40 bp, Artificial Sequence
  synthetic primer
tctatatgctccacagtatgcgagatgcccgcgctgatgg

SEQ ID NO: 389 - DNA, 43 bp, Artificial Sequence
  synthetic primer
actatatgctccactctatgcctaggcactggtcgaggagatg

SEQ ID NO: 390 - DNA, 18 bp, Artificial Sequence
  capture probe
tagccgcggtgtcgcctg

SEQ ID NO: 391 - DNA, 18 bp, Artificial Sequence
  capture probe
tagccgcgctgtcgcctg

SEQ ID NO: 392 - DNA, 21 bp, Artificial Sequence
  capture probe
cacgatggtgtcgtagaccgc

SEQ ID NO: 393 - DNA, 21 bp, Artificial Sequence
  capture probe
cacgatggtgccgtagaccgc

SEQ ID NO: 394 - DNA, 24 bp, Artificial Sequence
  capture probe
ctgaagtggatgttgctgaaggtc

SEQ ID NO: 395 - DNA, 19 bp, Artificial Sequence
  capture probe
cgtcttcggtagcggtcag

SEQ ID NO: 396 - DNA, 21 bp, Artificial Sequence
  capture probe
tcccaggtcccgcaggttcag

SEQ ID NO: 397 - DNA, 21 bp, Artificial Sequence
  capture probe
tcccaggtcccccaggttcag

SEQ ID NO: 398 - DNA, 20 bp, Artificial Sequence
  capture probe
ggatcttccgggtgatcagt

SEQ ID NO: 399 - DNA, 20 bp, Artificial Sequence
  capture probe
ggatcttccaggtgatcagt

SEQ ID NO: 400 - DNA, 18 bp, Artificial Sequence
  capture probe
cgctggtccctggggttg

SEQ ID NO: 401 - DNA, 18 bp, Artificial Sequence
  capture probe
cgctggtccgtggggttg

SEQ ID NO: 402 - DNA, 95 bp, Artificial Sequence; identical to SEQ ID NO: 339
  synthetic spacer

SEQ ID NO: 403 - DNA, 40 bp, Artificial Sequence
  synthetic primer
gctatgctcacagatgcgacgtctctggatggtatgcccg

SEQ ID NO: 404 - DNA, 40 bp, Artificial Sequence
  synthetic primer
gctatgctcacagatgcgacgtccgcctgcaagtcctaag

SEQ ID NO: 405 - DNA, 42 bp, Artificial Sequence
  synthetic primer
gctatgctcacagatgcgacgtacactataaaagcaccgtcg

SEQ ID NO: 406 - DNA, 42 bp, Artificial Sequence
  synthetic primer
gctatgctcacagatgcgacgtactattaccaacaaagctgc

SEQ ID NO: 407 - DNA, 40 bp, Artificial Sequence
  synthetic primer
gctatgctcacagatgcgacgaggtgcgatgatggcttct

SEQ ID NO: 408 - DNA, 42 bp, Artificial Sequence
  synthetic primer
gctatgctcacagatgcgacggtcttcatttggagcattcgg

SEQ ID NO: 409 - DNA, 41 bp, Artificial Sequence
  synthetic primer
gctatgctcacagatgcgacgacagagtgagtactttgacc

SEQ ID NO: 410 - DNA, 40 bp, Artificial Sequence
  synthetic primer
gctatgctcacagatgcgacggactcctcggtcaatatcc

SEQ ID NO: 411 - DNA, 39 bp, Artificial Sequence
  synthetic primer
gctatgctcacagatgcgacgtgacttctggacaaaccg

SEQ ID NO: 412 - DNA, 42 bp, Artificial Sequence
  synthetic primer
gctatgctcacagatgcgacgaacttcaaaccgaactatgac

SEQ ID NO: 413 - DNA, 40 bp, Artificial Sequence
  synthetic primer
gctatgctcacagatgcgacgggttgggcactaaaggcag

SEQ ID NO: 414 - DNA, 41 bp, Artificial Sequence
  synthetic primer
gctatgctcacagatgcgacgggagatggttggatattagg

SEQ ID NO: 415 - DNA, 41 bp, Artificial Sequence
  synthetic primer
gctatgctcacagatgcgacgataaacttcatcgcaccgtc

SEQ ID NO: 416 - DNA, 40 bp, Artificial Sequence
  synthetic primer
gctatgctcacagatgcgacgggcggcacatttgtcactg

SEQ ID NO: 417 - DNA, 41 bp, Artificial Sequence
  synthetic primer
gctatgctcacagatgcgacgaacgctgctcactagatgtc

SEQ ID NO: 418 - DNA, 41 bp, Artificial Sequence
  synthetic primer
gctatgctcacagatgcgacgtgtgactttatcgccattcc

SEQ ID NO: 419 - DNA, 41 bp, Artificial Sequence
  synthetic primer
gctatgctcacagatgcgacgtgtcctgcattaaaagctcc

SEQ ID NO: 420 - DNA, 41 bp, Artificial Sequence
  synthetic primer
gctatgctcacagatgcgacgaacatacatcgcagcaatcc

SEQ ID NO: 421 - DNA, 42 bp, Artificial Sequence
  synthetic primer
gctatgctcacagatgcgacgataccatccatatatttggga

SEQ ID NO: 422 - DNA, 41 bp, Artificial Sequence
  synthetic primer
gctatgctcacagatgcgacgccgagctggtcatagatgaa

SEQ ID NO: 423 - DNA, 42 bp, Artificial Sequence
  synthetic primer
gctatgctcacagatgcgacgattctggagtttcatctaaat

SEQ ID NO: 424 - DNA, 40 bp, Artificial Sequence
  synthetic primer
gctatgctcacagatgcgacgttccaactgccgtgacagc

SEQ ID NO: 425 - DNA, 40 bp, Artificial Sequence
  synthetic primer
gctatgctcacagatgcgacggcgataaaagcagacagcg

SEQ ID NO: 426 - DNA, 41 bp, Artificial Sequence
  synthetic primer
gctatgctcacagatgcgacgggaccagcagttgtagatac

SEQ ID NO: 427 - DNA, 23 bp, Artificial Sequence
  capture probe
gccggtattattgatgcggatgc

SEQ ID NO: 428 - DNA, 25 bp, Artificial Sequence
  capture probe
cttatcgatttcatccgcgtgtttc

SEQ ID NO: 429 - DNA, 26 bp, Artificial Sequence
  capture probe
cggtattgtcagatatttatgactca

SEQ ID NO: 430 - DNA, 26 bp, Artificial Sequence
  capture probe
cttgttaactaccgatgctgcattcg

SEQ ID NO: 431 - DNA, 23 bp, Artificial Sequence
  capture probe
tctggagcacttccatgaccacc

SEQ ID NO: 432 - DNA, 23 bp, Artificial Sequence
  capture probe
gcttgtgatcctccgctgccacc

SEQ ID NO: 433 - DNA, 25 bp, Artificial Sequence
  capture probe
gcaagaggaactcagacgggatttg

SEQ ID NO: 434 - DNA, 25 bp, Artificial Sequence
  capture probe
cttatcgatttcatccgcgtgtttc

SEQ ID NO: 435 - DNA, 27 bp, Artificial Sequence
  capture probe
tctattttcacgacttcaggctcaaaa

SEQ ID NO: 436 - DNA, 25 bp, Artificial Sequence
  capture probe
gagatgggcgtgaaactcaactatc

SEQ ID NO: 437 - DNA, 24 bp, Artificial Sequence
  capture probe
aatcctcatgatgccgagcccgca

SEQ ID NO: 438 - DNA, 28 bp, Artificial Sequence
  capture probe
atggatttggaataactataggagaaca

SEQ ID NO: 439 - DNA, 95 bp, Artificial Sequence; identical to SEQ ID NO: 339
  synthetic spacer

SEQ ID NO: 440 - DNA, 20 bp, Artificial Sequence
  synthetic primer
tgctatgctcacagatgcga

SEQ ID NO: 441 - DNA, 20 bp, Artificial Sequence
  synthetic primer
tgctatgctcacagatgcga

SEQ ID NO: 442 - DNA, 21 bp, Artificial Sequence
  synthetic primer
tgacctaagggactcccacaa

SEQ ID NO: 443 - DNA, 21 bp, Artificial Sequence
  synthetic primer
gtacaacggagcccagaacac

SEQ ID NO: 444 - DNA, 23 bp, Artificial Sequence
  synthetic primer
caaaggagctcactgtggtgtct

SEQ ID NO: 445 - DNA, 26 bp, Artificial Sequence
  synthetic primer
gggatatgagtcagaatggcttattc

SEQ ID NO: 446 - DNA, 24 bp, Artificial Sequence
  synthetic primer
tggatgaatatgaggagctcaatg

SEQ ID NO: 447 - DNA, 21 bp, Artificial Sequence
  synthetic primer
agcaggctggaatcagacaaa

SEQ ID NO: 448 - DNA, 21 bp, Artificial Sequence
  synthetic primer
ccctcccagtgtgcaaataag

SEQ ID NO: 449 - DNA, 25 bp, Artificial Sequence
  synthetic primer
ggacgtcgatggtattaggatagaa

SEQ ID NO: 450 - DNA, 22 bp, Artificial Sequence
  synthetic primer
aatacatggcttgctgcctgtt

SEQ ID NO: 451 - DNA, 19 bp, Artificial Sequence
  synthetic primer
cgtgcgcttggttttgatg

SEQ ID NO: 452 - DNA, 23 bp, Artificial Sequence
  synthetic primer
ttgacattgtggacatcggatac

SEQ ID NO: 453 - DNA, 22 bp, Artificial Sequence
  synthetic primer
aagttggaaggccggttaattt

SEQ ID NO: 454 - DNA, 402 bp, Artificial Sequence
  capture probe
ccttctccaccaaagacagttgcgcgcctgctccacgttctcttctgtctgttgcagcggaggcgtgcgtttgcttttacagatatctgaatggaagagtgtttcttccacaacagaagtatttctgtggatggcatcaaacagggcaaagtgttttttattgaatgcttataggttttttttaaataagtgggtcaagtacaccagccacctccagacaccagtgcgtgctcccgatgctgctatggaaggtgctacttgacctaagggactcccacaacaacaaaagcttgaagctgtggagggccacggtggcgtggctctcctcgcaggtgttctgggctccgttgtaccaagtggagcaggtggttgcgggcaagcgttgtgcagagcccatagcca

SEQ ID NO: 455 - DNA, 424 bp, Artificial Sequence
  capture probe
gccatccagcctgtcctttggaccacaccacccctccagcatggtcaccgccatgggttagagccctgctcgatgctcacagggcccccagcgagagtccctgcagtccctttcgacttgcatttttgcaggagcagtatcatgaagcctaaacgcgatggatatatgtttttgaaggcagaaagcaaaattatgtttgccactttgcaaaggagctcactgtggtgtctgtgttccaaccactgaatctggaccccatctgtgaataagccattctgactcatatcccctatttaacagggtctctagtgctgtgaaaaaaaaaaatcctgaacattgcatataacttatattgtaagaaatactgtacaatgactttattgcatctgggtagctgtaaggcatgaaggatgccaagaagttt

SEQ ID NO: 456 - DNA, 420 bp, Artificial Sequence
  capture probe
tgagaatggtggatgtggtggagaaagaagatgtgaatgaagccatcaggctaatggagatgtcaaaggactctcttctaggagacaaggggcagacagctaggactcagagaccagcagatgtgatatttgccaccgtccgtgaactggtctcagggggccgaagtgtccggttctctgaggcagagcagcgctgtgtatctcgtggcttcacacccgcccagttccaggcggctctggatgaatatgaggagctcaatgtctggcaggtcaatgcttcccggacacggatcacttttgtctgattccagcctgcttgcaaccctggggtcctcttgttccctgctggcctgccccttgggaaggggcagtgatgcctttgaggggaaggaggagcccctctttctcccatgctgcact

SEQ ID NO: 457 - DNA, 428 bp, Artificial Sequence
  capture probe
ggagcagagaggaggcaatggccaccatggagaacaaggtgatctgcgccctggtcctggtgtccatgctggccctcggcaccctggccgaggcccagacagagacgtgtacagtggccccccgtgaaagacagaattgtggttttcctggtgtcacgccctcccagtgtgcaaataagggctgctgtttcgacgacaccgttcgtggggtcccctggtgcttctatcctaataccatcgacgtccctccagaagaggagtgtgaattttagacacttctgcagggatctgcctgcatcctgacggggtgccgtccccagcacggtgattagtcccagagctcggctgccacctccaccggacacctcagacacgcttctgcagctgtgcctcggctcacaacacagattgactgctctgactttgac

SEQ ID NO: 458 - DNA, 461 bp, Artificial Sequence
  capture probe
gccaaccagatggtgaaatgtgaccctggccatggtaaatacatggcttgctgcctgttgtaccgtggtgacgtggttcccaaagatgtcaatgctgccattgccaccatcaaaaccaagcgcacgatccagtttgtggattggtgccccactggcttcaaggttggcatcaactaccagcctcccactgtggtgcctggtggagacctggccaaggtacagagagctgtgtgcatgctgagcaacaccacagccattgctgaggcctgggctcgcctggaccacaagtttgacctgatgtatgccaagcgtgcctttgttcactggtacgtgggtgaggggatggaggaaggcgagttttcagaggcccgtgaagatatggctgcccttgagaaggattatgaggaggttggtgtggattctgttgaaggagagggtgaggaagaaggagaggaatacta

SEQ ID NO: 459 - DNA, 482 bp, Artificial Sequence
  capture probe
gacagcacgctaataatgcaattactgagagacaacttgacattgtggacatcggatacccaaggagacgaagctgaagcaggagaaggaggggaaaattaaccggccttccaacttttgtctgcctcattctaaaatttacacagtagaccatttgtcatccatgctgtcccacaaatagttttttgtttacgatttatgacaggtttatgttacttctatttgaatttctatatttcccatgtggtttttatgtttaatattaggggagtagagccagttaacatttagggagttatctgttttcatcttgaggtggccaatatggggatgtggaatttttatacaagttataagtgtttggcatagtacttttggtacattgtggcttcaaaagggccagtgtaaaactgcttccatgtctaagcaaagaaaactgcctacatactggatttgtccctggcggggaataaaagggatca

SEQ ID NO: 460 - DNA, 20 bp, Artificial Sequence
  synthetic primer
tgctatgctcacagatgcga

SEQ ID NO: 461 - DNA, 22 bp, Artificial Sequence
  synthetic primer
ttgcacctttcactggtctcaa

SEQ ID NO: 462 - DNA, 23 bp, Artificial Sequence
  synthetic primer
gctagcaatggaagtgatgtcaa

SEQ ID NO: 463 - DNA, 23 bp, Artificial Sequence
  synthetic primer
actgctgagaactttgccaactt

SEQ ID NO: 464 - DNA, 21 bp, Artificial Sequence
  synthetic primer
ccagctaatgccaaaggatca

SEQ ID NO: 465 - DNA, 25 bp, Artificial Sequence
  synthetic primer
aaagggcattacttgaatgctactg

SEQ ID NO: 466 - DNA, 22 bp, Artificial Sequence
  synthetic primer
gaacgcccaattctctagcaaa

SEQ ID NO: 467 - DNA, 24 bp, Artificial Sequence
  synthetic primer
actggtaacgacttctccacattg

SEQ ID NO: 468 - DNA, 22 bp, Artificial Sequence
  synthetic primer
tccctagttggtgcccagtaga

SEQ ID NO: 469 - DNA, 30 bp, Artificial Sequence
  synthetic primer
ggtgtaaagataagtgatgtaacgtatgaa

SEQ ID NO: 470 - DNA, 24 bp, Artificial Sequence
  synthetic primer
tccactacatggatttgttttgct

SEQ ID NO: 471 - DNA, 23 bp, Artificial Sequence
  synthetic primer
tgttcaattcaggaggagcaatc

SEQ ID NO: 472 - DNA, 20 bp, Artificial Sequence
  synthetic primer
cgcgaaccttcatggaaact

SEQ ID NO: 473 - DNA, 491 bp, Artificial Sequence
  capture probe
cacacgcagcaatgttacacaagctagcatggttgcacctttcactggtctcaaatcttcagccactttccctgttacaaagaagcaaaaccttgacatcacttccattgctagcaatggtggaagagttagctgcatgcaggtgtggccacctattaacatgaagaagtacgagacactctcataccttcccgatttgtccgacgagcaattgcttagtgaaattgagtaccttttgaaaaatggatgggttccttgcttggaatttgagactgagcacggatttgtctaccgtgagaacaacaagtcaccaggatactatgatggaaggtactggaccatgtggaagttgcctatgtttgggtgcactgatgcaacccaagtgttggctgaggttcaagaggctaaaaaggcatacccacaagcatgggtcagaatcattggattcgacaatgtgcgtcaagtgcagtgtatcagtttcattgcctaca

SEQ ID NO: 474 - DNA, 404 bp, Artificial Sequence
  capture probe
ttgttggtcaagcctggagtggcattccatggtttgaggctggtgctgatcctggcgctattgcacctttctctttcggctcacttcttggcactcagcttctcctcatgggttgggttgagagcaaaaggtgggtcgacttcttcgacaatgactctcagtctatagattgggccactccatggtccaagactgctgagaactttgccaacttcacaggcgaacagggttaccctggtggcaaattctttgatcctttggcattagctggtacacttaacaatggagtttacgtccctgacacagagaagcttgagagattaaagcttgctgagatcaagcattctagacttgctatgttagccatgttaattttctattttgaggctggacaagggaagaca

SEQ ID NO: 475 - DNA, 422 bp, Artificial Sequence
  capture probe
aactcacaaccatttatgcgttggagagatcgtttcttattttgtgccgaagcactttwtaaagcacagactgaaacaggtgaaatcaaagggcattacttgaatgctactgcaggtacatgcgaagaaatgatcaaaagagctgtatttgctagagaattgggcgttccgatcgtaatgcatgactacttaacggggggatttaccgcaaatactaccttggctcattattgccgagataatggtctacttcttcacatccaccgtgcaatgcatgcggttattgatagacagaagaatcatggtatccacttccgggtattagcaaaagcgttacgtatgtctggtggagatcatattcactctggtaccgtagtaggtaaacttgaaggtgaaagagacataactttgggctttgttga

SEQ ID NO: 476 - DNA, 426 bp, Artificial Sequence
  capture probe
gcaaaggtacagagaggcagctgaaatcatcaggaaaggaaacatgtgttgtctcttcatcaacgatctcgatgcaggagctggtagaatgggtggaactacccaatacaccgtcaacaaccagatggtgaatgccaccctcatgaacattgctgacaacccaacaaatgtccagctccccggtatgtacaacaagcaagagaacgccagggtacccattattgtcactggtaacgacttctccacattgtatgctcctcttatccgtgatggtcgtatggagaagttctactgggcaccaactagggaggatagaattggtgtttgcaagggtattttcagaactgacaacgtccctgaggaagctgttgtaaagattgtcgattccttccctggacaatctattgatttctttggtgctttgag

SEQ ID NO: 477 - DNA, 402 bp, Artificial Sequence
  capture probe
aaatgtgacagttaagatggttagcttcacaagtactgagaatggtgtgagagtaaaaacatgggcaagacctagcaatggttttgttagaaatgttttatttcaacatattgttatgagtaatgttcaaaatccaataatcatagatcaaaattattgtcctaatcatgaaagttgtcctaatcagggctcaggtgtaaagataagtgatgtaacgtatgaagacatacatggaacatcagctacagaaatcgcggtgaaattagattgtagcaaaacaaatccatgtagtggaataacacttgaagatgtgaatcttagttataaaaatggtagagctgaagcttcatgtgttaatgctggaggaagagcttctggttttgaagaacttagtaaatgctt

SEQ ID NO: 478 - DNA, 415 bp, Artificial Sequence
  capture probe
  misc_feature 171: n = A,T,C or G
tgatgtcaattacttaccaaggattgctcatgatggatggaccggcgatgccattctgtattctcatcttcatagggaattgatcaatcttcctaaaaatacttcgattccaatcactctaaacgcgagagaatatgaagtctttacagttgttccaatcaatgaaatgtntacaggatcaagatttgctccgattggtcttgtgaatatgttcaattcaggaggagcaatcaaagaggtgaaatatgaaacagagggaaaatgtggactagtttccatgaaggttcgcggatgtggaacgtttggagcttattcatctgggaagcctaaacgaattcatgttgacaacgaagaagtacagtttgattacgacgaatcctctggattgttcaccattaacattacagttcctgat

SEQ ID NO: 479 - DNA, 431 bp, Artificial Sequence
  synthetic probe
agcacagaagatgggaacactggtggaggatggaaaggctcgctcaatcaagaaaattctgagactattaataaataagactgtagtgtagatactgagtaaatccatgcacctaaaccttttggaaaatctgccgtgggccctccagatagctcatttcattaagtttttccctccaaggtagaatttgcaagagtgacagtggattgcatttcttttggggaagctttcttttggtggttttgtttattataccttcttaagttttcaaccaaggtttgcttttgttttgagttactggggttatttttgttttaaataaaaataagtgtacaataagtgtttttgtattgaaagcttttgttatcaagattttcatacttttaccttccatggctctttttaagattgatacttttaagaggtggctg

SEQ ID NO: 480 - DNA, 402 bp, Artificial Sequence; identical to SEQ ID NO: 454
  synthetic probe

SEQ ID NO: 481 - DNA, 379 bp, Artificial Sequence
  synthetic probe
ccatcgtcagtgtgtgtgtttagagctgtgcaccctagaaacaacatacttgtcccatgagcaggtgcctgagacacagacccctttgcattcacagagaggtcattggttatagagacttgaattaataagtgacattatgccagtttctgttctctcacaggtgataaacaatgctttttgtgcactacatactcttcagtgtagagctcttgttttatgggaaaaggctcaaatgccaaattgtgtttgatggattaatatgcccttttgccgatgcatactattactgatgtgactcggttttgtcgcagctttgctttgtttaatgaaacacacttgtaaacctcttttgcactttgaaaaagaatccagcggg

SEQ ID NO: 482 - DNA, 424 bp, Artificial Sequence; identical to SEQ ID NO: 455
  synthetic probe

SEQ ID NO: 483 - DNA, 455 bp, Artificial Sequence
  synthetic probe
gtatggtaacttctctgagcttcagtttccaagtgaatttccatgtaataggacattcccattaaatacaagctgtttttactttttcgcctcccagggcctgtgggatctggtcccccagcctctcttgggctttcttacactaactctgtacctaccatctcctgcctcccttaggcaggcacctccaaccaccacacactccctgctgttttccctgcctggaactttccctcctgccccaccaagatcatttcatccagtcctgagctcagcttaagggaggcttcttgcctgtgggttccctcacccccatgcctgtcctccaggctggggcaggttcttagtttgcctggaattgttctgtacctctttgtagcacgtagtgttgtggaaactaagccactaattgagtttctggctcccctcctggggttgtaagttttgttcattca

SEQ ID NO: 484 - DNA, 390 bp, Artificial Sequence
  synthetic probe
gctgttctactaaaggctggcatgaccgttaagcaggctgtcctttataatgcattgtcagccatgctggcgtatcttggaatggcaacaggaattttcattggtcattatgctgaaaatgtttctatgtggatatttgcacttactgctggcttattcatgtatgttgctctggttgatatggtacctgaaatgctgcacaatgatgctagtgaccatggatgtagccgctgggggtatttctttttacagaatgctgggatgcttttgggttttggaattatgttacttatttccatatttgaacataaaatcgtgtttcgtataaatttctagttaaggtttaaatgctagagtagcttaaaaagttgtcatagtttcagtaggtca

SEQ ID NO: 485 - DNA, 420 bp, Artificial Sequence; identical to SEQ ID NO: 456
  synthetic probe

SEQ ID NO: 486 - DNA, 421 bp, Artificial Sequence
  synthetic probe
ctgtcattatggtgtccttacctgtgggagctgtaaggtcttctttaagagggcaatggaagggcagcacaactacttatgtgctggaagaaatgactgcatcgttgataaaatccgcagaaaaaactgcccagcatgtcgccttagaaagtgctgtcaggctggcatggtccttggaggtcgaaaatttaaaaagttcaataaagtcagagttgtgagagcactggatgctgttgctctcccacagccagtgggcgttccaaatgaaagccaagccctaagccagagattcactttttcaccaggtcaagacatacagttgattccaccactgatcaacctgttaatgagcattgaaccagatgtgatctatgcaggacatgacaacacaaaacctgacacctccagttctttgctgaca

SEQ ID NO: 487 - DNA, 428 bp, Artificial Sequence; identical to SEQ ID NO: 457
  synthetic probe

SEQ ID NO: 488 - DNA, 392 bp, Artificial Sequence
  synthetic probe
ctccacccagcaccttcaaagggacacctacggcagagaacccagagtacctgggtctggacgtgccagtgtgaaccagaaggccaagtccgcagaagccctgatgtgtcctcagggagcagggaaggcctgacttctgctggcatcaagaggtgggagggccctccgaccacttccaggggaacctgccatgccaggaacctgtcctaaggaaccttccttcctgcttgagttcccagatggctggaaggggtccagcctcgttggaagaggaacagcactggggagtctttgtggattctgaggccctgcccaatgagactctagggtccagtggatgccacagcccagcttggccctttccttccagatcctgggtactgaaagcctta

SEQ ID NO: 489 - DNA, 410 bp, Artificial Sequence
  synthetic probe
ttgactattacactgcctggaggatagcagagaagcctgtctgtacttcattcaaaaagccaaaatagagagtatacagtcctagagaattcctctatttgttcagatctcatagatgacccccaggtattgtcttttgacatccagcagtccaaggtattgagacatattactggaagtaagaaatattactataattgagaactacagcttttaagattgtacttttatcttaaaagggtggtagttttccctaaaatacttattatgtaagggtcattagacaaatgtcttgaagtagacatggaatttatgaatggttctttatcatttctcttccccctttttggcatcctggcttgcctccagttttaggtcctttagtttgcttctgtaagcaacgggaacac

SEQ ID NO: 490 - DNA, 461 bp, Artificial Sequence; identical to SEQ ID NO: 458
  synthetic probe

SEQ ID NO: 491 - DNA, 491 bp, Artificial Sequence
  synthetic probe
cgctgctgtcatcaaggctcgaaaactatccagtgccatgtctgctgcaaaagccatctgtgaccacgtcagggacatctggtttggaaccccagagggagagtttgtgtccatgggtgttatctctgatggcaactcctatggtgttcctgatgatctgctctactcattccctgttgtaatcaagaataagacctggaagtttgttgaaggtctccctattaatgatttctcacgtgagaagatggatcttactgcaaaggaactgacagaagaaaaagaaagtgcttttgaatttctttcctctgcctgactagacaatgatgttactaaatgcttcaaagctgaagaatctaaatgtcgtctttgactcaagtaccaaataataataatgctatacttaaattacttgtgaaaaacaacacattttaaagattacgtgcttcttggtacaggtttgtgaatgacagtttatcgtcatgctgttagtg

SEQ ID NO: 492 - DNA, 427 bp, Artificial Sequence
  synthetic probe
cgtgtgaagtgtagtggcatccatttctaatgtatgcattcatccaacagagttatttattggctggagatggaaaatcacaccacctgacaggccttctgggcctccaaagcccatccttggggttccccctccctgtgtgaaatgtattatcaccagcagacactgccgggcctccctcccgggggcactgcctgaaggcgagtgtgggcatagcattagctgcttcctcccctcctggcacccactgtggcctggcatcgcatcgtggtgtgtcaatgccacaaaatcgtgtgtccgtggaaccagtcctagccgcgtgtgacagtcttgcattctgtttgtctcgtggggggaggtggacagtcctgcggaaatgtgtcttgtcttccatttggataaaaggaaccaaccaacaaacaatgcc